SEQUENCE LISTING (182 sequences)

SEQ ID NO: 1 - DNA, 681 bp, Vaccinia virus
  gene (1)..(681)
atgttgtcgatgtttatgtgtaataatatcgtagattatgtagatgatatagataatggtatagtacaggatatagaagatgaggctagcaataatgttgatcacgactatgtatatccacttccagaaaatatggtatatagatttgacaagtccactaacatactcgattatctatcaacggaacgggaccatgtaatgatggctgttcgatactatatgagtaaacaacgtttagacgacttgtatagacagttgcccacaaagactagatcatatatagatattatcaacatatattgtgataaagttagtaatgattataatagggacatgaatatcatgtatgatatggcatctacaaaatcatttacagtttatgacataaataacgaagttaatactatactaatggataacaaggggttgggtgtaagattggcgacaatttcatttataaccgaattgggtagacgatgtatgaacccagtagaaacgataaaaatgtttactctactatcgcatactatatgcgatgattattttgtagattatataacggacatttcaccaccagataataccatccctaacactagcacgcgtgaatatctaaagcttattggcatcacagctatcatgtttgctacatataaaactctcaaatacatgataggataa

SEQ ID NO: 2 - protein, 226 aa, Vaccinia virus
  PEPTIDE (1)..(226)
MLSMFMCNNIVDYVDDIDNGIVQDIEDEASNNVDHDYVYPLPENMVYRFDKSTNILDYLSTERDHVMMAVRYYMSKQRLDDLYRQLPTKTRSYIDIINIYCDKVSNDYNRDMNIMYDMASTKSFTVYDINNEVNTILMDNKGLGVRLATISFITELGRRCMNPVETIKMFTLLSHTICDDYFVDYITDISPPDNTIPNTSTREYLKLIGITAIMFATYKTLKYMIG

SEQ ID NO: 3 - protein, 205 aa, Vaccinia virus
  PEPTIDE (1)..(205)
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 4 - protein, 239 aa, Vaccinia virus
  PEPTIDE (1)..(239)
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 5 - protein, 11 aa, Human immunodeficiency virus
  PEPTIDE (1)..(11)
GRKKRRQRPPQ

SEQ ID NO: 6 - protein, 16 aa, Drosophila melanogaster
  PEPTIDE (1)..(16)
RQIKIWFQNRRMKWKK

SEQ ID NO: 7 - protein, 16 aa, Drosophila melanogaster
  PEPTIDE (1)..(16)
RQIAIWFQNRRMKWAA

SEQ ID NO: 8 - protein, 9 aa, Human immunodeficiency virus
  PEPTIDE (1)..(9)
RKKRRQRRR

SEQ ID NO: 9 - protein, 21 aa, Artificial Sequence
  Buforin II - antimicrobial peptide derived from Histone 2a
  PEPTIDE (1)..(21)
TRSSRAGLQFPVGRVHRLLRK

SEQ ID NO: 10 - protein, 26 aa, Artificial Sequence
  Transportan - transporter peptide
  PEPTIDE (1)..(26)
GWTLNSAGYLLGKINKALAALAKKIL

SEQ ID NO: 11 - protein, 18 aa, Artificial Sequence
  Model amphipathic peptide (MAP) - transporter peptide
  PEPTIDE (1)..(18)
KLALKLALKALKAALKLA

SEQ ID NO: 12 - protein, 16 aa, Artificial Sequence
  K-FGF - transporter peptide
  PEPTIDE (1)..(16)
AAVALLPAVLLALLAP

SEQ ID NO: 13 - protein, 10 aa, Artificial Sequence
  Ku-70 - transporter peptide
  PEPTIDE (1)..(10)
VPMLKPMLKE

SEQ ID NO: 14 - protein, 28 aa, Artificial Sequence
  prion - transporter peptide
  PEPTIDE (1)..(28)
MANLGYWLLALFVTMWTDVGLCKKRPKP

SEQ ID NO: 15 - protein, 18 aa, Artificial Sequence
  pVEC - transporter peptide
  PEPTIDE (1)..(18)
LLIILRRRIRKQAHAHSK

SEQ ID NO: 16 - protein, 21 aa, Artificial Sequence
  Pep-1 - transporter peptide
  PEPTIDE (1)..(21)
KETWWETWWTEWSQPKKKRKV

SEQ ID NO: 17 - protein, 18 aa, Artificial Sequence
  SynB1 -  internalization transporter peptide
  PEPTIDE (1)..(18)
RGGRLSYSRRRFSTSTGR

SEQ ID NO: 18 - protein, 15 aa, Artificial Sequence
  Pept-7 -  internalization transporter peptide
  PEPTIDE (1)..(15)
SDLWEMMMVSLACQY

SEQ ID NO: 19 - protein, 12 aa, Artificial Sequence
  HN-1 -  internalization transporter peptide
  PEPTIDE (1)..(12)
TSPLNIHNGQKL

SEQ ID NO: 20 - RNA, 25 nt, Artificial Sequence
  probe
  misc_feature (1)..(25)
uacuguuugucaugccacuucugau

SEQ ID NO: 21 - protein, 9 aa, Artificial Sequence
  synthetic - transporter peptide
  PEPTIDE (1)..(9)
RRRRRRRRR

SEQ ID NO: 22 - protein, 16 aa, Artificial Sequence
  Antp - internalization transporter peptide
  PEPTIDE (1)..(16)
RQPKIWFPNRRKPWKK

SEQ ID NO: 23 - protein, 25 aa, Vaccinia virus
  PEPTIDE (1)..(25)
CNNIVDYVDDIDNGIVQDIEDEASN

SEQ ID NO: 24 - protein, 25 aa, Vaccinia virus
  PEPTIDE (1)..(25)
DYVDDIDNGIVQDIEDEASNNVDHD

SEQ ID NO: 25 - protein, 25 aa, Vaccinia virus
  PEPTIDE (1)..(25)
IDNGIVQDIEDEASNNVDHDYVYPL

SEQ ID NO: 26 - protein, 26 aa, Vaccinia virus
  PEPTIDE (1)..(26)
VQDIEDEASNNVDHDYVYPLPENMVY

SEQ ID NO: 27 - protein, 21 aa, Vaccinia virus
  PEPTIDE (1)..(21)
VQDIEDEASNNVDHDYVYPLP

SEQ ID NO: 28 - protein, 16 aa, Vaccinia virus
  PEPTIDE (1)..(16)
VQDIEDEASNNVDHDY

SEQ ID NO: 29 - protein, 11 aa, Vaccinia virus
  PEPTIDE (1)..(11)
VQDIEDEASNN

SEQ ID NO: 30 - protein, 11 aa, Vaccinia virus
  PEPTIDE (1)..(11)
DEASNNVDHDY

SEQ ID NO: 31 - protein, 14 aa, Vaccinia virus
  PEPTIDE (1)..(14)
VQDIEDEASNNVDH

SEQ ID NO: 32 - protein, 13 aa, Vaccinia virus
  PEPTIDE (1)..(13)
VQDIEDEASNNVD

SEQ ID NO: 33 - protein, 12 aa, Vaccinia virus
  PEPTIDE (1)..(12)
VQDIEDEASNNV

SEQ ID NO: 34 - protein, 6 aa, Vaccinia virus
  PEPTIDE (1)..(6)
NVDHDY

SEQ ID NO: 35 - protein, 6 aa, Artificial Sequence
  Synthetic F1L peptide
  PEPTIDE (1)..(6): X can be any amino acid
NVXHXY

SEQ ID NO: 36 - protein, 6 aa, Artificial Sequence
  Synthetic F1L peptide
  peptide (1)..(6): X at position 3 can be any amino acid and X at position 5 can be D, E, A, G, V, L, F, I, W, or P
NVXHXY

SEQ ID NO: 37 - protein, 6 aa, Artificial Sequence
  Synthetic F1L peptide
  peptide (1)..(6): X at position 3 can be any amino acid and X at position 5 can be D, E, A, G, V or L
NVXHXY

SEQ ID NO: 38 - protein, 6 aa, Artificial Sequence
  Synthetic F1L peptide
  peptide (1)..(6): X at position 3 can be any amino acid and X at position 5 can be D, E, A or G
NVXHXY

SEQ ID NO: 39 - protein, 6 aa, Artificial Sequence
  Synthetic Bcl-2 Loop peptide
  peptide (1)..(6): X at position 3 can be any amino acid and X at position 5 can be D or E
NVXHXY

SEQ ID NO: 40 - protein, 6 aa, Artificial Sequence
  Synthetic F1L peptide
  peptide (1)..(6): X at position 3 can be any amino acid
NVXHDY

SEQ ID NO: 41 - protein, 6 aa, Artificial Sequence
  Synthetic F1L peptide
  peptide (1)..(6): X can be any amino acid
NVXHAY

SEQ ID NO: 42 - protein, 6 aa, Artificial Sequence
  Synthetic F1L peptide
  peptide (1)..(6): X at position 3 can be D,E, A, G,V, L, F, I, W or P and X at position 5 can be any amino acid
NVXHXY

SEQ ID NO: 43 - protein, 6 aa, Artificial Sequence
  Synthetic F1L peptide
  peptide (1)..(6): X at position 3 can be D, E, A, G, V or L and X at position 5 can by any amino acid
NVXHXY

SEQ ID NO: 44 - protein, 6 aa, Artificial Sequence
  Synthetic F1L peptide
  peptide (1)..(6): X at position 3 can be D, E, A or G and X at position 5 can be any amino acid
NVXHXY

SEQ ID NO: 45 - protein, 6 aa, Artificial Sequence
  Synthetic F1L peptide
  peptide (1)..(6): X at position 3 can be D or E and X at position 5 can be any amino acid
NVXHXY

SEQ ID NO: 46 - protein, 6 aa, Artificial Sequence
  Synthetic F1L peptide
  peptide (1)..(6): X can be any amino acid
NVDHXY

SEQ ID NO: 47 - protein, 6 aa, Artificial Sequence
  Synthetic F1L peptide
  peptide (1)..(6): X can be any amino acid
NVAHXY

SEQ ID NO: 48 - protein, 6 aa, Artificial Sequence
  Synthetic F1L peptide
  PEPTIDE (1)..(6)
NVXHXY

SEQ ID NO: 49 - protein, 6 aa, Artificial Sequence
  Synthetic F1L peptide
  peptide (1)..(6): X at position 3 can be D, E, A, G, V, L, F, I, W or P and X at position 5 can be D, E, A, G, V, or L
NVXHXY

SEQ ID NO: 50 - protein, 6 aa, Artificial Sequence
  Synthetic F1L peptide
  peptide (1)..(6): X at position 3 can be D, E, A, G, V, L, F, I, W or P and X at position 5 can be D, E, A, or G
NVXHXY

SEQ ID NO: 51 - protein, 6 aa, Artificial Sequence
  Synthetic F1L peptide
  peptide (1)..(6): X at position 3 can be D, E, A, G, V, L, F, I, W, or P and X at position 5 can be D or E
NVXHXY

SEQ ID NO: 52 - protein, 6 aa, Artificial Sequence
  Synthetic F1L peptide
  peptide (1)..(6): X can be any amino acid
NVXHDY

SEQ ID NO: 53 - protein, 6 aa, Artificial Sequence
  Synthetic F1L peptide
  peptide (1)..(6): X can be D, E, A, G, V, L, F, I, W, or P
NVXHAY

SEQ ID NO: 54 - protein, 6 aa, Artificial Sequence
  Synthetic F1L peptide
  peptide (1)..(6): X at position 3 can be D, E, A, G, V, or L and X at position 5 can be D, E, A, G, V, L, F, I, W or P
NVXHXY

SEQ ID NO: 55 - protein, 6 aa, Artificial Sequence
  Synthetic F1L peptide
  PEPTIDE (1)..(6): X at position 3 can be D, E, A, G, V, or L and X at position 5 can be D, E, A, G, V, or L
NVXHXY

SEQ ID NO: 56 - protein, 6 aa, Artificial Sequence
  Synthetic F1L peptide
  peptide (1)..(6): X at position 3 can be D, E, A, G, V or L and X at position 5 can be D, E, A or G
NVXHXY

SEQ ID NO: 57 - protein, 6 aa, Artificial Sequence
  Synthetic F1L peptide
  peptide (1)..(6): X at position 3 can be D, E, A, G, V or L and X at position 5 can be D or E
NVXHXY

SEQ ID NO: 58 - protein, 6 aa, Artificial Sequence
  Synthetic F1L peptide
  peptide (1)..(6): X can be D, E, A, G, V or L
NVXHDY

SEQ ID NO: 59 - protein, 6 aa, Artificial Sequence
  Synthetic F1L peptide
  peptide (1)..(6): X can be D, E, A, G, V or L
NVXHAY

SEQ ID NO: 60 - protein, 6 aa, Artificial Sequence
  Synthetic F1L peptide
  peptide (1)..(6): X at position 3 can be D, E, A or G and X at position 5 can be D, E, A, G, V, L, F, I, W or P
NVXHXY

SEQ ID NO: 61 - protein, 6 aa, Artificial Sequence
  Synthetic F1L peptide
  peptide (1)..(6): X at position 3 can be D, E, A or G and X at position 5 can be D, E, A, G, V or L
NVXHXY

SEQ ID NO: 62 - protein, 6 aa, Artificial Sequence
  Synthetic F1L peptide
  peptide (1)..(6): X at position 3 can be D, E, A or G and X at position 5 can be D, E, A or G
NVXHXY

SEQ ID NO: 63 - protein, 6 aa, Artificial Sequence
  Synthetic F1L peptide
  peptide (1)..(6): X at position 3 can be D, E, A or G and X at position 5 can be D or E
NVXHXY

SEQ ID NO: 64 - protein, 6 aa, Artificial Sequence
  Synthetic F1L peptide
  peptide (1)..(6): X can be D, E, A or G
NVXHDY

SEQ ID NO: 65 - protein, 6 aa, Artificial Sequence
  Synthetic F1L peptide
  peptide (1)..(6): X can be D, E, A or G
NVXHAY

SEQ ID NO: 66 - protein, 6 aa, Artificial Sequence
  Synthetic F1L peptide
  peptide (1)..(6): X at position 3 can be D or E and X at position 5 can be D, E, A, G, V, L, F, I, W or P
NVXHXY

SEQ ID NO: 67 - protein, 6 aa, Artificial Sequence
  Synthetic F1L peptide
  peptide (1)..(6): X at position 3 can be D or E and X at position 5 can be D, E, A, G, V or L
NVXHXY

SEQ ID NO: 68 - protein, 6 aa, Artificial Sequence
  Synthetic F1L peptide
  peptide (1)..(6): X at position 3 can be D or E and X at position 5 can be D, E, A or G
NVXHXY

SEQ ID NO: 69 - protein, 6 aa, Artificial Sequence
  Synthetic F1L peptide
  peptide (1)..(6): X at position 3 can be D or E and X at position 5 can be D or E
NVXHXY

SEQ ID NO: 70 - protein, 6 aa, Artificial Sequence
  Synthetic F1L peptide
  peptide (1)..(6): X can be D or E
NVXHDY

SEQ ID NO: 71 - protein, 6 aa, Artificial Sequence
  Synthetic F1L peptide
  peptide (1)..(6): X can be D or E
NVXHAY

SEQ ID NO: 72 - protein, 6 aa, Artificial Sequence
  Synthetic F1L peptide
  peptide (1)..(6): X can be D, E, A, G, V, L, F, I, W or P
NVDHXY

SEQ ID NO: 73 - protein, 6 aa, Artificial Sequence
  Synthetic F1L peptide
  peptide (1)..(6): X can be D, E, A, G, V, or L
NVDHXY

SEQ ID NO: 74 - protein, 6 aa, Artificial Sequence
  Synthetic F1L peptide
  peptide (1)..(6): X can be D, E, A or G
NVDHXY

SEQ ID NO: 75 - protein, 6 aa, Artificial Sequence
  Synthetic F1L peptide
  peptide (1)..(6): X can be D or E
NVDHXY

SEQ ID NO: 76 - protein, 6 aa, Artificial Sequence
  Synthetic F1L peptide
  peptide (1)..(6)
NVDHAY

SEQ ID NO: 77 - protein, 6 aa, Artificial Sequence
  Synthetic F1L peptide
  peptide (1)..(6): X at position 3 can be D, E, A, G, V or L and X at position 5 can be D, E, A, G, V, L, F, I, W or P
NVXHXY

SEQ ID NO: 78 - protein, 6 aa, Artificial Sequence
  Synthetic F1L peptide
  peptide (1)..(6): X at position 3 can be D, E, A, or G and X at position 5 can be D, E, a, G, V, L, F, I, W or P
NVXHXY

SEQ ID NO: 79 - protein, 6 aa, Artificial Sequence
  Synthetic F1L peptide
  peptide (1)..(6): X at position 3 can be D or E and X at position 5 can be D, E, a, G, V, L, F, I, W or P
NVXHXY

SEQ ID NO: 80 - protein, 6 aa, Artificial Sequence
  Synthetic F1L peptide
  peptide (1)..(6): X can be D, E, A, G, V, L, F, I, W or P
NVDHXY

SEQ ID NO: 81 - protein, 6 aa, Artificial Sequence
  Synthetic F1L peptide
  peptide (1)..(6): X can be D, E, A, G, V, L, F, I, W or P
NVAHXY

SEQ ID NO: 82 - protein, 6 aa, Artificial Sequence
  Synthetic F1L peptide
  peptide (1)..(6): X at position 3 can be D, E, A, G, V, L, F, I, W or P and X at position 5 can be D, E, A, G, V or L
NVXHXY

SEQ ID NO: 83 - protein, 6 aa, Artificial Sequence
  Synthetic F1L peptide
  peptide (1)..(6): X at position 3 can be D, E, A, G, V or L and X at position 5 can be D, E, A, G, V or L
NVXHXY

SEQ ID NO: 84 - protein, 6 aa, Artificial Sequence
  Synthetic F1L peptide
  peptide (1)..(6): X at position 3 can be D, E, A or G and X at position 5 can be D, E, A, G, V or L
NVXHXY

SEQ ID NO: 85 - protein, 6 aa, Artificial Sequence
  Synthetic F1L peptide
  PEPTIDE (1)..(6)
NVXHXY

SEQ ID NO: 86 - protein, 6 aa, Artificial Sequence
  Synthetic F1L peptide
  peptide (1)..(6): X can be D, E, A, G, V or L
NVDHXY

SEQ ID NO: 87 - protein, 6 aa, Artificial Sequence
  Synthetic F1L peptide
  peptide (1)..(6): X can be D, E, A, G, V or L
NVAHXY

SEQ ID NO: 88 - protein, 6 aa, Artificial Sequence
  Synthetic F1L peptide
  peptide (1)..(6): X at position 3 can be D, E, A, G, V, L, F, I, W or P and X at position 5can be D, E, A, or G
NVXHXY

SEQ ID NO: 89 - protein, 6 aa, Artificial Sequence
  Synthetic F1L peptide
  peptide (1)..(6): X at position 3 can be D, E, A, G, V or L and X at position 5 can be D, E, A or G
NVXHXY

SEQ ID NO: 90 - protein, 6 aa, Artificial Sequence
  Synthetic F1L peptide
  peptide (1)..(6): X at position 3 can be D, E, A, or G and X at position 5 can be D, E, A or G
NVXHXY

SEQ ID NO: 91 - protein, 6 aa, Artificial Sequence
  Synthetic F1L peptide
  peptide (1)..(6): X at position 3 can be D or E and X at position 5 can be D, E, A, or G
NVXHXY

SEQ ID NO: 92 - protein, 6 aa, Artificial Sequence
  Synthetic F1L peptide
  peptide (1)..(6): X can be D, E, A or G
NVDHXY

SEQ ID NO: 93 - protein, 6 aa, Artificial Sequence
  Synthetic F1L peptide
  peptide (1)..(6): X can be D, E, A or G
NVAHXY

SEQ ID NO: 94 - protein, 6 aa, Artificial Sequence
  Synthetic F1L peptide
  peptide (1)..(6): X at position 3 can be D, E, A, G, V, L, F, I, W or P and X at position 5 can be D or E
NVXHXY

SEQ ID NO: 95 - protein, 6 aa, Artificial Sequence
  Synthetic F1L peptide
  peptide (1)..(6): X at position 3 can be D, E, A, G, V or L and X at position 5 can be D or E
NVXHXY

SEQ ID NO: 96 - protein, 6 aa, Artificial Sequence
  Synthetic F1L peptide
  peptide (1)..(6): X at position 3 can be D, E, A or G and X at position 5 can be D or E
NVXHXY

SEQ ID NO: 97 - protein, 6 aa, Artificial Sequence
  Synthetic F1L peptide
  peptide (1)..(6): X at position 3 can be D or E and X at position 5 can be D or E
NVXHXY

SEQ ID NO: 98 - protein, 6 aa, Artificial Sequence
  Synthetic F1L peptide
  peptide (1)..(6): X can be D or E
NVDHXY

SEQ ID NO: 99 - protein, 6 aa, Artificial Sequence
  Synthetic F1L peptide
  peptide (1)..(6): X can be D or E
NVAHXY

SEQ ID NO: 100 - protein, 6 aa, Artificial Sequence
  Synthetic F1L peptide
  peptide (1)..(6): X can be D, E, A, G, V, L, F, I, W, or P
NVXHDY

SEQ ID NO: 101 - protein, 6 aa, Artificial Sequence
  Synthetic F1L peptide
  peptide (1)..(6): X can be D, E, A, G, V or L
NVXHDY

SEQ ID NO: 102 - protein, 6 aa, Artificial Sequence
  Synthetic F1L peptide
  peptide (1)..(6): X can be D, E, A or G
NVXHDY

SEQ ID NO: 103 - protein, 6 aa, Artificial Sequence
  Synthetic F1L peptide
  peptide (1)..(6): X can be D or E
NVXHDY

SEQ ID NO: 104 - protein, 6 aa, Artificial Sequence
  Synthetic F1L peptide
  peptide (1)..(6)
NVAHDY

SEQ ID NO: 105 - protein, 6 aa, Artificial Sequence
  Synthetic F1L peptide
  peptide (1)..(6)
NVAHAY

SEQ ID NO: 106 - protein, 6 aa, Vaccinia virus
  peptide (1)..(6)
AVDHDY

SEQ ID NO: 107 - protein, 6 aa, Vaccinia virus
  peptide (1)..(6)
NADHDY

SEQ ID NO: 108 - protein, 6 aa, Vaccinia virus
  peptide (1)..(6)
NVDADY

SEQ ID NO: 109 - protein, 6 aa, Vaccinia virus
  peptide (1)..(6)
NVDHDA

SEQ ID NO: 110 - protein, 25 aa, Artificial Sequence
  labeled peptide - Synthetic F1L peptide with FAM label
  peptide (1)..(25)
CNNIVDYVDDIDNGIVQDIEDEASN

SEQ ID NO: 111 - protein, 25 aa, Artificial Sequence
  labeled peptide - Synthetic F1L peptide with FAM label
  peptide (1)..(25)
DYVDDIDNGIVQDIEDEASNNVDHD

SEQ ID NO: 112 - protein, 25 aa, Artificial Sequence
  labeled peptide - Synthetic F1L peptide with FAM label
  peptide (1)..(25)
IDNGIVQDIEDEASNNVDHDYVYPL

SEQ ID NO: 113 - protein, 26 aa, Artificial Sequence
  labeled peptide - Synthetic F1L peptide with FAM label
  peptide (1)..(26)
VQDIEDEASNNVDHDYVYPLPENMVY

SEQ ID NO: 114 - protein, 10 aa, Artificial Sequence
  Synthetic Bcl-2 Loop peptide
  peptide (1)..(10)
PLQTPAAPGA

SEQ ID NO: 115 - protein, 10 aa, Artificial Sequence
  Synthetic Bcl-2 Loop peptide
  peptide (1)..(10): X can be any amino acid
PXXTPXXXXX

SEQ ID NO: 116 - protein, 10 aa, Artificial Sequence
  Synthetic Bcl-2 Loop peptide
  peptide (1)..(10): X can be any amino acid
PXXTPAAXXX

SEQ ID NO: 117 - protein, 10 aa, Artificial Sequence
  Synthetic Bcl-2 Loop peptide
  peptide (1)..(10): X at position 2 can be L, A, G, I, V, F, W or P and X at positions 3 and 6-10 can be any amino acid
PXXTPXXXXX

SEQ ID NO: 118 - protein, 10 aa, Artificial Sequence
  Synthetic Bcl-2 Loop peptide
  peptide (1)..(10): X at position 2 can be L, A, G, I, or V and X at positions 3 and 6-10 can be any amino acid
PXXTPXXXXX

SEQ ID NO: 119 - protein, 10 aa, Artificial Sequence
  Synthetic Bcl-2 Loop peptide
  peptide (1)..(10): X at position 2 can be L, A, or G and X at positions 3 and 6-10 can be any amino acid
PXXTPXXXXX

SEQ ID NO: 120 - protein, 10 aa, Artificial Sequence
  Synthetic Bcl-2 Loop peptide
  peptide (1)..(10): X can be any amino acid
PLXTPXXXXX

SEQ ID NO: 121 - protein, 10 aa, Artificial Sequence
  Synthetic Bcl-2 Loop peptide
  peptide (1)..(10): X can be any amino acid
PAXTPXXXXX

SEQ ID NO: 122 - protein, 10 aa, Artificial Sequence
  Synthetic Bcl-2 Loop peptide
  peptide (1)..(10): X at position 2 can be L, A, G, I, V, F, W or P and X at position 3 and 6-10 can be any amino acid
PXXTPAAXXX

SEQ ID NO: 123 - protein, 10 aa, Artificial Sequence
  Synthetic Bcl-2 Loop peptide
  peptide (1)..(10): X at position 2 can be L, A, G, I, or V and X at position 3 and 6-10 can be any amino acid
PXXTPAAXXX

SEQ ID NO: 124 - protein, 10 aa, Artificial Sequence
  Synthetic Bcl-2 Loop peptide
  peptide (1)..(10): X at position 2 can be L, A or G and X at positions 3 and 6-10 can be any amino acid
PXXTPAAXXX

SEQ ID NO: 125 - protein, 10 aa, Artificial Sequence
  Synthetic Bcl-2 Loop peptide
  peptide (1)..(10): X can be any amino acid
PLXTPAAXXX

SEQ ID NO: 126 - protein, 10 aa, Artificial Sequence
  Synthetic Bcl-2 Loop peptide
  peptide (1)..(10): X can be any amino acid
PAXTPAAXXX

SEQ ID NO: 127 - protein, 10 aa, Artificial Sequence
  Synthetic Bcl-2 Loop peptide
  peptide (1)..(10): X at position 2 can be Q, N, R, A, G, V, L, F, I, W or P and X at positions 3 and 6-10 can be any amino acid
PXXTPXXXXX

SEQ ID NO: 128 - protein, 10 aa, Artificial Sequence
  Synthetic Bcl-2 Loop peptide
  peptide (1)..(10): X at position 2 can be Z, N, R, A, G, V or L and X at position 3 and 6-10 can be any amino acid
PXXTPXXXXX

SEQ ID NO: 129 - protein, 10 aa, Artificial Sequence
  Synthetic Bcl-2 Loop peptide
  peptide (1)..(10): X at position 2 can be Q, N, R, A or G and X at position 3 and 6-10 can be any amino acid
PXXTPXXXXX

SEQ ID NO: 130 - protein, 10 aa, Artificial Sequence
  Synthetic Bcl-2 Loop peptide
  peptide (1)..(10): X at position 2 can be Q, N or R and X at position 3 and 6-10 can be any amino acid
PXXTPXXXXX

SEQ ID NO: 131 - protein, 10 aa, Artificial Sequence
  Synthetic Bcl-2 Loop peptide
  peptide (1)..(10): X can be any amino acid
PXQTPXXXXX

SEQ ID NO: 132 - protein, 10 aa, Artificial Sequence
  Synthetic Bcl-2 Loop peptide
  peptide (1)..(10): X can be any amino acid
PXATPXXXXX

SEQ ID NO: 133 - protein, 10 aa, Artificial Sequence
  Synthetic Bcl-2 Loop peptide
  peptide (1)..(10): X at postion 2 can be Q, N, R, a, G, V, L, F, I, W or P and X at positions 3 and 8-10 can be any amino acid
PXXTPAAXXX

SEQ ID NO: 134 - protein, 10 aa, Artificial Sequence
  Synthetic Bcl-2 Loop peptide
  peptide (1)..(10): X at position 2 can be Q, N, R, A, G, V or L and X at positions 3 and 8-10 can be any amino acid
PXXTPAAXXX

SEQ ID NO: 135 - protein, 10 aa, Artificial Sequence
  Synthetic Bcl-2 Loop peptide
  peptide (1)..(10): X at position 2 can be Q, N, R, A or G and X at postions 3 and 8-10 can be any amino acid
PXXTPAAXXX

SEQ ID NO: 136 - protein, 10 aa, Artificial Sequence
  Synthetic Bcl-2 Loop peptide
  peptide (1)..(10): X at position 2 can be Q, N or R and X at position 3 and 8-10 can be any amino acid
PXXTPAAXXX

SEQ ID NO: 137 - protein, 10 aa, Artificial Sequence
  Synthetic Bcl-2 Loop peptide
  peptide (1)..(10): X can be any amino acid
PXQTPAAXXX

SEQ ID NO: 138 - protein, 10 aa, Artificial Sequence
  Synthetic Bcl-2 Loop peptide
  peptide (1)..(10): X can be any amino acid
PXATPAAXXX

SEQ ID NO: 139 - protein, 10 aa, Artificial Sequence
  Synthetic Bcl-2 Loop peptide
  peptide (1)..(10): X at postiion 2, 3 and 7-10 can be any amino acid and X at postion 6 can be A, G, V, L, F, I, W, or P
PXXTPXXXXX

SEQ ID NO: 140 - protein, 10 aa, Artificial Sequence
  Synthetic Bcl-2 Loop peptide
  peptide (1)..(10): X at positions 2, 3 and 7-10 can be any amino acid and X at position 6 can be A, G, V or L
PXXTPXXXXX

SEQ ID NO: 141 - protein, 10 aa, Artificial Sequence
  Synthetic Bcl-2 Loop peptide
  peptide (1)..(10): X at position 2, 3 and 7-10 can be any amino acid and X at position 6 can be A or G
PXXTPXXXXX

SEQ ID NO: 142 - protein, 10 aa, Artificial Sequence
  Synthetic Bcl-2 Loop peptide
  peptide (1)..(10): X can be any amino acid
PXXTPAXXXX

SEQ ID NO: 143 - protein, 10 aa, Artificial Sequence
  Synthetic Bcl-2 Loop peptide
  peptide (1)..(10): X at position 2, 3, 6 and 8-10 can be any amino acid and X at position 7 can be A, G, V, L, F, I, W or P
PXXTPXXXXX

SEQ ID NO: 144 - protein, 10 aa, Artificial Sequence
  Synthetic Bcl-2 Loop peptide
  peptide (1)..(10): X at position 2, 3, 6 and 8-10 can be any amino acid and X at position 7 can be A, G, V or L
PXXTPXXXXX

SEQ ID NO: 145 - protein, 10 aa, Artificial Sequence
  Synthetic Bcl-2 Loop peptide
  peptide (1)..(10): X at position 2, 3, 6, and 8-10 can be any amino acid and X at position 7 can be A or G
PXXTPXXXXX

SEQ ID NO: 146 - protein, 10 aa, Artificial Sequence
  Synthetic Bcl-2 Loop peptide
  peptide (1)..(10): X can be any amino acid
PXXTPXAXXX

SEQ ID NO: 147 - protein, 10 aa, Artificial Sequence
  Synthetic Bcl-2 Loop peptide
  peptide (1)..(10): X at positions 2, 3, 6, 7, 9 and 10 can be any amino acid and X at position 8 can be P, A, G, V, L, F, I or W
PXXTPXXXXX

SEQ ID NO: 148 - protein, 10 aa, Artificial Sequence
  Synthetic Bcl-2 Loop peptide
  peptide (1)..(10): X at positions 2, 3, 6, 7, 9 and 10 can be any amino acid and X at position 8 can be P, A, G, V or L
PXXTPXXXXX

SEQ ID NO: 149 - protein, 10 aa, Artificial Sequence
  Synthetic Bcl-2 Loop peptide
  peptide (1)..(10): X at positions 2, 3, 6, 7, 9 and 10 can be any amino acid and X at position 8 can be P, A, or G
PXXTPXXXXX

SEQ ID NO: 150 - protein, 10 aa, Artificial Sequence
  Synthetic Bcl-2 Loop peptide
  peptide (1)..(10): X can be any amino acid
PXXTPXXPXX

SEQ ID NO: 151 - protein, 10 aa, Artificial Sequence
  Synthetic Bcl-2 Loop peptide
  peptide (1)..(10): X can be any amino acid
PXXTPXXAXX

SEQ ID NO: 152 - protein, 10 aa, Artificial Sequence
  Synthetic Bcl-2 Loop peptide
  peptide (1)..(10): X at positions 2,3, 6-8 and 10 can be any amino acid and X at position 9 can be G, A, V, L, F, I, W or P
PXXTPXXXXX

SEQ ID NO: 153 - protein, 10 aa, Artificial Sequence
  Synthetic Bcl-2 Loop peptide
  peptide (1)..(10): X at positions 2, 3, 6-8 and 10 can be any amino acid and X at position 9 can be G, A, V or L
PXXTPXXXXX

SEQ ID NO: 154 - protein, 10 aa, Artificial Sequence
  Synthetic Bcl-2 Loop peptide
  peptide (1)..(10): X at positions 2, 3, 6-8 and 10 can be any amino acid and X at position 9 can be G or A
PXXTPXXXXX

SEQ ID NO: 155 - protein, 10 aa, Artificial Sequence
  Synthetic Bcl-2 Loop peptide
  peptide (1)..(10): X can be any amino acid
PXXTPXXXGX

SEQ ID NO: 156 - protein, 10 aa, Artificial Sequence
  Synthetic Bcl-2 Loop peptide
  peptide (1)..(10): X can be any amino acid
PXXTPXXXAX

SEQ ID NO: 157 - protein, 10 aa, Artificial Sequence
  Synthetic Bcl-2 Loop peptide
  peptide (1)..(10): X at positions 2, 3, 6-19 can be any amino acid and X at position 10 can be A, G, V, L, F, I, W, or P
PXXTPXXXXX

SEQ ID NO: 158 - protein, 10 aa, Artificial Sequence
  Synthetic Bcl-2 Loop peptide
  peptide (1)..(10): X at positions 2, 3 and 6-9 can be any amino acid and X at position 10 can be A, G, V or L
PXXTPXXXXX

SEQ ID NO: 159 - protein, 10 aa, Artificial Sequence
  Synthetic Bcl-2 Loop peptide
  peptide (1)..(10): X at positions 2, 3, 6-9 can be any amino acids and X at positions 10 can be A or G
PXXTPXXXXX

SEQ ID NO: 160 - protein, 10 aa, Artificial Sequence
  Synthetic Bcl-2 Loop peptide
  peptide (1)..(10): X can be any amino acid
PXXTPXXXXA

SEQ ID NO: 161 - protein, 49 aa, Artificial Sequence
  Synthetic Bcl-2 Loop peptide
  peptide (1)..(49)
VGAAPPGAAPAPGIFSSQPGHTPHPAASRDPVARTSPLQTPAAPGAAAG

SEQ ID NO: 162 - protein, 22 aa, Artificial Sequence
  Synthetic Bcl-2 Loop peptides
  peptide (1)..(22)
YDNREIVMKYIHYKLSQRGYEW

SEQ ID NO: 163 - protein, 20 aa, Artificial Sequence
  Synthetic Bcl-2 Loop peptide
  peptide (1)..(20)
DAGDVGAAPPGAAPAPGIFS

SEQ ID NO: 164 - protein, 20 aa, Artificial Sequence
  Synthetic Bcl-2 Loop peptide
  peptide (1)..(20)
GAAPAPGIFSSQPGHTPHPA

SEQ ID NO: 165 - protein, 20 aa, Artificial Sequence
  Synthetic Bcl-2 Loop peptide
  peptide (1)..(20)
SQPGHTPHPAASRDPVARTS

SEQ ID NO: 166 - protein, 20 aa, Artificial Sequence
  Synthetic Bcl-2 Loop peptide
  peptide (1)..(20)
ASRDPVARTSPLQTPAAPGA

SEQ ID NO: 167 - protein, 20 aa, Artificial Sequence
  Synthetic Bcl-2 Loop peptide
  peptide (1)..(20)
PLQTPAAPGAAAGPALSPVP

SEQ ID NO: 168 - protein, 15 aa, Artificial Sequence
  Synthetic Bcl-2 Loop peptide
  peptide (1)..(15)
PLQTPAAPGAAAGPA

SEQ ID NO: 169 - protein, 15 aa, Artificial Sequence
  Synthetic Bcl-2 Loop peptide
  peptide (1)..(15)
AAPGAAAGPALSPVP

SEQ ID NO: 170 - protein, 10 aa, Artificial Sequence
  Synthetic Bcl-2 Loop peptide
  peptide (1)..(10)
AAGPALSPVP

SEQ ID NO: 171 - protein, 5 aa, Artificial Sequence
  Synthetic Bcl-2 Loop peptide
  peptide (1)..(5)
PLQTP

SEQ ID NO: 172 - protein, 10 aa, Artificial Sequence
  Synthetic Bcl-2 Loop peptide
  peptide (1)..(10)
ALQTPAAPGA

SEQ ID NO: 173 - protein, 10 aa, Artificial Sequence
  Synthetic Bcl-2 Loop Peptide
  peptide (1)..(10)
PAQTPAAPGA

SEQ ID NO: 174 - protein, 10 aa, Artificial Sequence
  Synthetic Bcl-2 Loop peptide
  peptide (1)..(10)
PLATPAAPGA

SEQ ID NO: 175 - protein, 10 aa, Artificial Sequence
  Synthetic Bcl-2 Loop peptide
  peptide (1)..(10)
PLQAPAAPGA

SEQ ID NO: 176 - protein, 10 aa, Artificial Sequence
  Synthetic Bcl-2 Loop peptide
  peptide (1)..(10)
PLQTAAAPGA

SEQ ID NO: 177 - protein, 10 aa, Artificial Sequence
  Synthetic Bcl-2 Loop peptide
  peptide (1)..(10)
PLQTPAAAGA

SEQ ID NO: 178 - protein, 10 aa, Artificial Sequence
  Synthetic Bcl-2 Loop peptide
  peptide (1)..(10)
PLQTPAAPAA

SEQ ID NO: 179 - protein, 10 aa, Artificial Sequence
  Synthetic Bcl-2 Loop peptide
  peptide (1)..(10)
PLQTPGAPGA

SEQ ID NO: 180 - protein, 10 aa, Artificial Sequence
  Synthetic Bcl-2 Loop peptide
  peptide (1)..(10)
PLQTPAGPGA

SEQ ID NO: 181 - protein, 10 aa, Artificial Sequence
  Synthetic Bcl-2 Loop peptide
  peptide (1)..(10)
PLQTPAAPGG

SEQ ID NO: 182 - protein, 6 aa, Artificial Sequence
  Synthetic F1L peptide
  peptide (1)..(6)
NVDHAY

(19) United States
(12) Patent Application Publication
(10) Pub. No.: US 2010/0093623 A1
(43) Pub. Date: Apr. 15, 2010

(54) COMPOSITIONS AND METHODS FOR MODULATING NOD-LIKE RECEPTOR ACTIVITY AND USES THEREOF

(75) Inventors: John C. Reed, Rancho Santa Fe, CA (US); Benjamin Faustin, La Jolla, CA (US); Arnold Satterthwait, La Jolla, CA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/504,649

(22) Filed: Jul. 16, 2009

Related U.S. Application Data
(60) Provisional application No. 61/081,350, filed on Jul. 16, 2008.

(51) Int. Cl. A61K 38/16; C07K 7/06; C07K 7/08; C07K 14/00; A61P 31/04; C12N 9/96; A61K 38/08; A61K 38/10; A61P 29/00; G01N 33/566; G01N 33/53

(52) U.S. Cl. 514/12; 530/329; 530/326; 530/324; 530/328; 435/188; 514/17; 514/13; 514/15; 436/501; 435/7.9; 435/7.5; 435/7.92

(57) ABSTRACT

Disclosed herein are compositions and methods relating to a peptide that inhibits Nod-like Receptors. Further provided are compositions and methods for treating or preventing inflammation, including diseases associated with inflammation such as inflammatory bowel disease, Crohn's disease, ulcerative colitis, arthritis, psoriasis, Alzheimer's disease, cardiovascular disease, diabetes, and sepsis.

### CROSS-REFERENCE TO RELATED APPLICATIONS

This application claims benefit of U.S. Provisional Application No. 61/081,350, filed Jul. 16, 2008. Application No. 61/081,350, filed Jul. 16, 2008, is hereby incorporated herein by reference in its entirety.

### STATEMENT REGARDING FEDERALLY SPONSORED RESEARCH

This invention was made with government support under Grants AI055789 and AI056324 awarded by the National Institutes of Health. The government has certain rights in the invention.

### REFERENCE TO SEQUENCE LISTING

The Sequence Listing submitted Nov. 25, 2009 as a text file named “24520_{—}7_{—}8402_{—}2009_{—}11_{—}25_Sequence_Listing_ST25.txt,” created on Nov. 24, 2009, and having a size of 60 kilobytes is hereby incorporated by reference pursuant to 37 C.F.R. §1.52(e)(5).

### BACKGROUND

NLR-family proteins (NOD-Like Receptors [NLRs]) are recently recognized components of innate immunity, constituting large families of related proteins that contain a nucleotide-binding oligomerization domain called NACHT and several leucine-rich repeat (LRR) domains involved in pathogen sensing (Ting et al., (2008) Immunity 28, 285-7; Mariathasan & Monack, (2007) Nat Rev Immunol 7, 31-40). Binding of pathogen-derived molecules to the LRRs is thought to induce conformational changes that allow NACHT-domain mediated oligomerization, thus initiating downstream signaling events, including activation of proteases involved in cytokine processing and activation. In this regard, the NACHT and LRRs are often associated with other domains that allow many of the NLRs to bind directly to pro-Caspase-1 (via CARD domains) or indirectly through adaptor proteins (via PYRIN domains). Caspase-1 belongs to the inflammatory group of Caspases, which cleave pro-Interleukin-1β (IL-1β), pro-IL-18, and pro-IL-33 (Salvesen, (2002) Essays Biochem 38, 9-19) in the cytosol, thus preparing them for secretion. Excessive activation of Caspase-1 can also induce cell death, either by apoptosis or by a variant recently termed “pyroptosis”, especially during host responses to pathogens (for review, see (Ting et al., (2008) Nat Rev Immunol 8, 372-9)).

The human genome contains at least 22 NLR-encoding genes. NLRP1 is among the best characterized, with the NLRP1 protein representing the central component of a multi-protein, Caspase-1-activating complex termed the “inflammasome.” (Martinon et al., (2002) Mol Cell 10, 417-26). Microbial ligands capable of activating NLRP1 include muramyl dipeptide (MDP), a component of peptidoglycans produced by gram-positive and gram-negative bacteria (Faustin et al., Mol. Cell. 2007 25(5):713-724; Bruey et al., Cell 2007 129(1):45-56). The murine NLRP1b isoform was reported to be crucial for anthrax lethal toxin-mediated macrophage cell killing (Boyden 2006), whereas the human ortholog is involved in tissue injury in the context of ultraviolet (UV)-irradiated keratinocytes (Feldmeyer et al., (2007) Curr Biol 17, 1140-5; Faustin & Reed, (2008) Trends Cell Biol 18, 4-8). In addition, hereditary polymorphisms in NLR-encoding genes are involved in several autoimmune and inflammatory diseases (Inohara et al., (2005) Annu Rev Biochem 74, 355-83), including NLRP1, which is associated with Vitiligo and related inflammatory diseases (Jin et al., (2007) N Engl J Med 356, 1216-25).

### BRIEF SUMMARY

In accordance with the purpose of this invention, as embodied and broadly described herein, this invention relates to compositions and methods relating to peptides that inhibit or activate Nod-like Receptors. Further provided are compositions and methods for treating or preventing, for example, inflammation, including diseases associated with inflammation such as inflammatory bowel disease, Crohn's disease, ulcerative colitis, arthritis, psoriasis, Alzheimer's disease, cardiovascular disease, diabetes, and sepsis.

Additional uses and advantages of the disclosed methods and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B and 1C shows immunoblots indicating that the viral Bcl-2 homolog F1L of vaccinia virus bind NLRs.

FIG. 2A shows immunoblots indicating that a portion of F1L (residues 1-44) are required to bind NLRP1.

FIG. 2B shows a graph of IL-1β production requires a portion of F1L (residues 1-44) to inhibit MDP-inducible IL-1β production in macrophages.

FIGS. 3A, 3B and 3C show graphs of caspase-1 activity indicating that a portion of recombinant F1L (residues 1-48), but not N1L, suppresses the in vitro reconstituted NLRP1 inflammasome.

FIGS. 4A, 4B, 4C and 4D show data indicating that synthetic F1L peptide (22-47) inhibits the ATP binding to NLRP1 as measured by caspase-1 activity (4A), processing of caspase-1 (4B), percent inhibition of ATP binding (4C) and unlabled F1L competition (4D).

FIGS. 5A, 5B and 5C show immunoblots indicating that F1L inhibits caspase-1 cleavage in vaccinia-infected macrophages.

FIGS. 6A, 6B and 6C show graphs of IL-1β, TNFα and IL-8 production indicating that F1L inhibits proIL-1β processing in vaccinia-infected macrophages.

FIGS. 7A, 7B, 7C and 7D show graphs of IL-1β and IL-8 production indicating that F1L inhibits proIL-1β processing in vaccinia-infected macrophages and PBMC.

FIG. 8 shows a graph of percent initial body weight of mice infected with a form of vaccinia virus indicating that F1L, and not N1L, is required for substantial weight loss.

FIGS. 9A and 9B show graphs of caspase-1 activity (RFU/min) for various peptides of F1L and Bcl-2.

FIG. 10 shows a graph of caspase-1 activity indicating that recombinant F1L (residues 32-37) provides maximum suppression of the in vitro reconstituted NLRP1 inflammasome.

FIG. 11 shows NLRP1 inflammasome components and Bcl-2 family proteins. Domain structures of NLRP1, pro-Caspase-1, and Bcl-2 family proteins used for the studies are depicted, showing Pyrin (PYD); NACHT (with Walker-A and B motifs forming the nucleotide binding site) NLRP1-associated domain (NAD); leucine-rich repeats (LRR); domain with unknown function (FIIND); Caspase-recruitment domain (CARD); Bcl-2 homology domain (BH) 1, 2, 3, and 4 (black rectangles), and loop domains are indicated in Bcl-2 and Bcl-XL proteins, which were expressed without their C-terminal transmembrane (ATM) domains.

FIGS. 12A, 12B and 12C show Bcl-2 and Bcl-X_{L} suppress in vitro reconstituted NLRP1 inflammasome. (A) Reactions contained His-NLRP1 (8.5 nM), pro-Caspase-1 (8.5 nM), 0.25 mM ATP, 0.5 mM Mg2+, 0.1 μg/ml MDP and GST-Bcl-2, or GST-Bcl-X_{L}, or His-Bcl-X_{L}ΔLoop, or GST, or Bid (17 nM). Caspase-1 activity was measured after 60 minutes by hydrolysis of Ac-WEHD-AMC substrate (20 μM), expressing data as mean±SD, n=3. Asterisks indicate p<0.05. (B) His6-NLRP1 with or without GST-Bcl-2, GST-Bcl-X_{L}, or His-Bcl-X_{L}ΔLoop (molar ratio: 1/5) was incubated 15 min in ice, then with pro-Caspase-1 in the presence of 1 mM ATP, 1 mM Mg2+, 20 μg/ml MDP for 30 mM at 37° C. Proteins were separated by SDS-PAGE and then immunoblotted using anti-p10 Caspase-1 antibodies. (C) Purified active p10/p20 Caspase-1 (5 nM) in presence or absence of GST-Bcl-2, or GST-Bcl-X_{L}, or Ac-WEHD-CHO was incubated with 10 mM DTT 10 min at 37° C. Caspase-1 activity was measured after 60 minutes by hydrolysis of Ac-WEHD-AMC substrate (20 μM), expressing data as mean±SD, n=3.

FIGS. 13A and 13B show enzymology of NLRP1 inflammasome inhibition by Bcl-2 and Bcl-X_{L}. (A) Reactions contained His-NLRP1 (8.5 nM), pro-Caspase-1 (8.5 nM), 0.25 mM ATP, 0.5 mM Mg2+, 0.1 μg/ml MDP and various concentrations of recombinant GST-Bcl-2 (black symbols) or GST-Bcl-X_{L} (white symbols). Caspase-1 activity was measured after 60 minutes by hydrolysis of Ac-WEHD-AMC substrate (20 μM), expressing data as mean±SD, n=3. (B) Reactions contained His-NLRP1 (8.5 nM), pro-caspase-1 (8.5 nM), 0.25 mM ATP, 0.5 mM Mg2+, 0.1 μg/ml MDP, without (black symbols) or with (white symbols) GST-Bcl-2 or GST-Bcl-X_{L} (2 nM). Caspase-1 activity was measured after 60 minutes by hydrolysis of various concentrations of Ac-WEHD-AMC substrate, expressing data as mean±SD, n=3. Experimental data points were analyzed by a linear regression method to fit the Lineweaver-Burk equation. Parameters in FIG. 17 are obtained from the data presented in (B) (mean±SD; n=3).

FIGS. 14A and 14B show Bcl-2 and Bcl-X_{L} inhibit ATP binding to NLRP1. (A) His6-NLRP1 (0.125 μM) was incubated for 15 min in ice in the presence of GST-Bcl-X_{L}, His-Bcl-X_{L}ΔLoop, GST-Bcl-2, GST, or Bid (1 μM). The mixture was then incubated for additional 15 min in ice with 1 μM MDP-LD, FITC-conjugated ATP analog (10 nM) and Mg2+ (0.5 mM). ATP binding was analyzed by FPA (n=3), measuring milliPolars [mP], and the percentage of inhibition was determined vs NLRP1 incubated only with MDP-LD (mean±SD). (B) His6-NLRP1 (0.1 μM) was incubated for 15 min in ice with various amounts of GST-Bcl-2 (black squares), GST-Bcl-X_{L} (white triangles), or GST-Bcl-X_{L}ΔLoop (black triangles) and then incubated 15 min on ice with 1 μM MDP-LD, FITC-conjugated ATP analog (10 nM) and Mg2+ (0.5 mM). ATP binding to NLRP1 was analyzed by FPA (n=3), measuring milliPolars [mP], and the percentage of inhibition calculated (mean±SD).

FIG. 15 shows Bcl-X_{L} inhibits oligomerization of NLRP1. Purified His6-NLRP1 monomers obtained from gel filtration were incubated 15 min in ice with or without GST-Bcl-X_{L}, His-Bcl-X_{L}ΔLoop, or GST (1/10 molar ratio). The mixture was then incubated without (no treatment [NT]) or with Mg2+ (1 mM), ATP (1 mM) and MDP-LD (20 μg/ml) for 30 min at 37° C. Proteins were separated by a first native-PAGE dimension, then by a second denaturating SDS-PAGE dimension, and stained using Sypro-Ruby. Arrows at bottom indicate positions of NLRP1 monomers and oligomers.

FIGS. 16A-16E show Bcl-2 Loop peptide inhibits NLRP1. (A) Reactions contained His6-NLRP1 (8.5 nM), pro-Caspase-1 (8.5 nM), 0.25 mM ATP, 0.5 mM Mg2+, 0.1 μg/ml MDP and Bcl-2 Loop peptide 35-83 (50 nM). Asterisk indicates p<0.05. (B) Purified active p10/p20 Caspase-1 (5 nM) was incubated with or without Bcl-2 Loop peptide 35-83 or Ac-WEHD-CHO (10 nM). (C) Reactions contained His6-NLRP1 (8.5 nM), pro-Caspase-1 (8.5 nM), 0.25 mM ATP, 0.5 mM Mg2+, 0.1 μg/ml MDP and various 20′mer Bcl-2 Loop peptides (50 nM), or Ac-WEHD-CHO (10 nM). Asterisks indicate p<0.05. (D) Reactions contained His6-NLRP1ΔLRR (8.5 nM), pro-Caspase-1 (8.5 nM), 0.25 mM ATP, 0.5 mM Mg2+, 0.1 μg/ml MDP and Bcl-2 Loop peptide 35-83, 71-90 peptide (50 nM), or Ac-WEHD-CHO (10 nM). (E) Reactions contained His6-NLRP1 (8.5 nM), pro-Caspase-1 (8.5 nM), 0.25 mM ATP, 0.5 mM Mg2+, 0.1 μg/ml MDP and various amounts of Bcl-2 Loop peptide 35-83, 71-90 Loop peptide, or BH4 peptide 9-30. For all experiments, Caspase-1 activity was measured after 60 minutes by hydrolysis of Ac-WEHD-AMC substrate (20 μM), expressing data as mean±SD, n=3.

FIG. 17 shows kinetic parameters of NLRP1 inflammasome inhibition by Bcl-2 and Bcl-X_{L}. Parameters are obtained from the data presented in FIG. 13B using a non-linear regression method to fit the Michaelis-Menten equation (mean±SD; n=3).

FIG. 18 shows alanine scanning of Bcl-2 71-80 and F1L 32-37 peptides. Reactions contained His6-NLRP1 (8.5 nM), pro-Caspase-1 (8.5 nM), 0.25 mM ATP, 0.5 mM Mg2+, 0.1 μg/ml MDP and various (A) Bcl-2 or (B) F1L Loop mutant peptides (50 nM) for which each amino acid was consecutively substituted by Alanine. Caspase-1 activity was measured after 60 minutes by hydrolysis of Ac-WEHD-AMC substrate (20 μM), expressing data as mean±SD, n=3.

FIG. 19 shows Bcl-2 and F1L peptides inhibit ATP binding to NLRP1. (A) ATP binding by NLRP1 was measured in presence or absence of peptides. Reactions contained His6-NLRP1 (0.125 μM), GST-Bcl-2 protein or Bcl-2 loop peptides (2 or 20 μM), 1 μM MDP[LD], FITC-ATP analog (10 nM) and Mg2+ (0.5 mM). FITC-ATP binding was analyzed by FPA (n=3), measuring milliPolars [mP], and values corrected for non-specific FITC-ATP binding as determined by competition with excess unlabeled ATP. The percentage inhibition was determined compared to NLRP1 incubated only with MDP-LD (mean±SD). (B) His6-NLRP1 (0.125 μM) was incubated for 5 min on ice in the presence of F1L 1-47 (2 μM), N1L, or F1L peptides 22-47 (0.5 or 2 μM) or 32-37, or Bcl-2 peptide 41-60 (2 μM). The mixture was then incubated for additional 5 min with 1 μM MDP, FL-conjugated ATP analog (10 nM) and Mg2+ (0.5 mM). ATP binding was analyzed by fluorescence polarization (n=3 milliPolars [mP]), and the percentage of inhibition was determined vs NLRP1 incubated only with MDP (mean±SD; n=3).

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Nod-Like Receptors (NLRs) constitute a recently described protein family involved in innate immunity by acting in the cytosol as pathogen sensors which results in inducing the host inflammatory response through Interleukin-1β (IL-1β) maturation and secretion. NLRs constitute the core of a macrocomplex called “inflammasome” that activates procaspase-1 which in turn maturates IL-1β to be secreted to mediate the acute phase of inflammation. Diverse inflammatory diseases have been characterized showing uncontrolled IL-1β secretion, and many diseases-associated mutations were evidenced for some members, such as NLRP1 for Vitiligo and associated diseases. Moreover, NLRP1 is involved in the susceptibility for macrophage cell death mediated by the Anthrax lethal toxin. Recently, proapoptotic proteins Bcl-2 and Bcl-XL were described to inhibit NLRP1-mediated caspase-1 activation. Some Nod-like Receptors have alternative names in the art. For example, NLRP1 and NLRP3 are also referred to as NALP1 and NALP3, respectively.

F1L has been identified as a Bcl-2 homolog. F1L peptide (residues 22-47) binds to NLRP1 and inhibits the ATP binding, subsequently suppressing NLRP1 inflammasome assembly. The disclosed peptides can be used to inhibit NLR activity, inflammasome activity, caspase-1 activity, production of IL-1β, IL-18 and IL-33, and inflammation and to identify small molecule inhibitors of NLR activity, inflammasome activity, caspase-1 activity, production of IL-1β, IL-18 and IL-33, and inflammation.

Inflammasomes can activate caspase-1. Inhibition of NLR by the disclosed compositions and methods can thus inhibit activation of caspase-1. Caspase-1 activates IL-1β, IL-18 and IL-33. Accordingly, modulation of caspase-1 activity can modulate the activity of IL-1β, IL-18 and IL-33, which in turn modulates systems and pathways affected by IL-1β, IL-18 and IL-33. For example, IL-1β is involved in inflammation and inflammatory processes and modulation of caspase-1 activity can affect inflammation and processes, conditions and diseases that involve inflammation. In this way, modulation of NLR activity can modulate the activity of IL-1β, IL-18 and IL-33, which in turn modulates systems and pathways affected by IL-1β, IL-18 and IL-33, such as inflammation. Caspase-1 can also stimulate apoptosis. Accordingly, modulation of caspase-1 activity can modulate apoptosis. In this way, modulation of NLR activity can modulate apoptosis.

The disclosed F1L peptides are the first peptide inhibitor ever identified for a NLR family member, like NLRP1. The disclosed peptides can inhibit inflammasome assembly, by blocking the ATP binding to NLR monomers.

The disclosed peptides can also be used for drug screening assays where peptide binding to NLR is measured and compounds that displace the peptide are detected.

The binding of the peptide to NLR can be measured by, for example, Fluorescence Polarization Assay (FPA) using fluorochome-conjugated peptide, Time Resolved Fluorescent Resonance Energy Transfer (TR-FRET), and Scintillation Proximity Assay (SPA). In addition, it is contemplated that the peptide can be used to treat cells, especially using peptides incorporating membrane-penetrating carrier sequences such as HIV-tat, Drosophila antennapedia protein, poly-L-arginine, poly-D-arginine, and poly-Arginine.

Disclosed are isolated peptides comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR.

Also disclosed are peptides comprising an amino acid sequence having at least 70% identity to the sequence set forth in SEQ ID NO:2 that can bind to a Nod-Like Receptor (NLR), or a fragment of the sequence set forth in SEQ ID NO:2 of at least 6 amino acids in length that can bind to a NLR. For example, the amino acid sequence can comprise amino acid residues 32-37 of SEQ ID NO:2. The amino acid sequence can comprise amino acid residues 27-37 of SEQ ID NO:2, or a fragment thereof of at least 6 amino acids. The amino acid sequence can comprise amino acid residues 22-37 of SEQ ID NO:2, or a fragment thereof of at least 6 amino acids. The amino acid sequence can comprise amino acid residues 22-47 of SEQ ID NO:2, or a fragment thereof of at least 6 amino acids. The amino acid sequence can consist of amino acid residues 22-47 of SEQ ID NO:2, or a fragment thereof of at least 6 amino acids. The amino acid sequence can consist of amino acid residues 22-37 of SEQ ID NO:2, or a fragment thereof of at least 6 amino acids. The amino acid sequence can consist of amino acid residues 32-37 of SEQ ID NO:2. The amino acid sequence can comprise or consist of amino acids 32-37 of SEQ ID NO:2, amino acids 32-37 of SEQ ID NO:2 where one or both of amino acids 34 and 36 can be substituted independently with any amino acid, or amino acids 71-80 of SEQ ID NO:3 where one or both of amino acids 34 and 36 can be substituted independently with E, A, G, V, L, F, I, W, or P.

Also disclosed are peptides comprising an amino acid sequence having at least 70% identity to the sequence set forth in SEQ ID NO:2 that can bind to a Nod-Like Receptor (NLR), or a fragment of the sequence set forth in SEQ ID NO:2 of at least 8 amino acids in length that can bind to a NLR. For example, the amino acid sequence can comprise amino acid residues 32-37 of SEQ ID NO:2. The amino acid sequence can comprise amino acid residues 27-37 of SEQ ID NO:2, or a fragment thereof of at least 8 amino acids. The amino acid sequence can comprise amino acid residues 22-37 of SEQ ID NO:2, or a fragment thereof of at least 8 amino acids. The amino acid sequence can comprise amino acid residues 22-47 of SEQ ID NO:2, or a fragment thereof of at least 8 amino acids. The amino acid sequence can consist of amino acid residues 22-47 of SEQ ID NO:2, or a fragment thereof of at least 8 amino acids. The amino acid sequence can consist of amino acid residues 22-37 of SEQ ID NO:2, or a fragment thereof of at least 8 amino acids. The amino acid sequence can consist of amino acid residues 32-37 of SEQ ID NO:2.

Also disclosed are peptides comprising an amino acid sequence having at least 70% identity to the sequence set forth in SEQ ID NO:3 or SEQ ID NO:4 that can bind to a Nod-Like Receptor (NLR), or a fragment of the sequence set forth in SEQ ID NO:3 or SEQ ID NO:4 of at least 6 amino acids in length that can bind to a NLR. For example, the amino acid sequence can comprise amino acid residues 71-83 of SEQ ID NO:3 or SEQ ID NO:4, or a fragment thereof of at least 6 amino acids. The amino acid sequence can comprise amino acid residues 71-90 of SEQ ID NO:3 or SEQ ID NO:4, or a fragment thereof of at least 6 amino acids. The amino acid sequence can comprise amino acid residues 60-90 of SEQ ID NO:3 or SEQ ID NO:4, or a fragment thereof of at least 6 amino acids. The amino acid sequence can comprise amino acid residues 35-90 of SEQ ID NO:3 or SEQ ID NO:4, or a fragment thereof of at least 6 amino acids. The amino acid sequence can consist of amino acid residues 35-90 of SEQ ID NO:3 or SEQ ID NO:4, or a fragment thereof of at least 6 amino acids. The amino acid sequence can consist of amino acid residues 60-90 of SEQ ID NO:3 or SEQ ID NO:4, or a fragment thereof of at least 6 amino acids. The amino acid sequence can consist of amino acid residues 71-90 of SEQ ID NO:3 or SEQ ID NO:4, or a fragment thereof of at least 6 amino acids. The amino acid sequence can consist of amino acid residues 71-83 of SEQ ID NO:3 or SEQ ID NO:4, or a fragment thereof of at least 6 amino acids. The amino acid sequence can comprise or consist of amino acids 71-80 of SEQ ID NO:3, amino acids 71-80 of SEQ ID NO:3 where one or more of amino acids 72, 73, 76, 77, 78, 79, and 80 can be substituted independently with any amino acid, or amino acids 71-80 of SEQ ID NO:3 where one or more of amino acids 72, 73, 76, 77, 78, 79, and 80 can be substituted independently with A, G, I, V, F, W, or P (for amino acid 72), N, R, A, G, V, L, F, I, W, or P (for amino acid 73), G, V, L, F, I, W, or P (for amino acid 76), G, V, L, F, I, W, or P (for amino acid 77), A, G, V, L, F, I, or W (for amino acid 78), A, V, L, F, I, or W (for amino acid 79), and G, V, L, F, I, W, or P (for amino acid 80).

Also disclosed are peptides comprising an amino acid sequence having at least 70% identity to the sequence set forth in SEQ ID NO:3 or SEQ ID NO:4 that can bind to a Nod-Like Receptor (NLR), or a fragment of the sequence set forth in SEQ ID NO:3 or SEQ ID NO:4 of at least 8 amino acids in length that can bind to a NLR. For example, the amino acid sequence can comprise amino acid residues 71-83 of SEQ ID NO:3 or SEQ ID NO:4, or a fragment thereof of at least 8 amino acids. The amino acid sequence can comprise amino acid residues 71-90 of SEQ ID NO:3 or SEQ ID NO:4, or a fragment thereof of at least 8 amino acids. The amino acid sequence can comprise amino acid residues 60-90 of SEQ ID NO:3 or SEQ ID NO:4, or a fragment thereof of at least 8 amino acids. The amino acid sequence can comprise amino acid residues 35-90 of SEQ ID NO:3 or SEQ ID NO:4, or a fragment thereof of at least 8 amino acids. The amino acid sequence can consist of amino acid residues 35-90 of SEQ ID NO:3 or SEQ ID NO:4, or a fragment thereof of at least 8 amino acids. The amino acid sequence can consist of amino acid residues 60-90 of SEQ ID NO:3 or SEQ ID NO:4, or a fragment thereof of at least 8 amino acids. The amino acid sequence can consist of amino acid residues 71-90 of SEQ ID NO:3 or SEQ ID NO:4, or a fragment thereof of at least 8 amino acids. The amino acid sequence can consist of amino acid residues 71-83 of SEQ ID NO:3 or SEQ ID NO:4, or a fragment thereof of at least 8 amino acids. The amino acid sequence can comprise amino acid residues 71-80 of SEQ ID NO:3. The amino acid sequence can consist of amino acid residues 71-80 of SEQ ID NO:3. The amino acid sequence can comprise amino acid residues 71-80, 71-90, 71-100, 65-80, 60-80, 60-90, or 60-100 of SEQ ID NO:3, or a fragment thereof of at least 6 amino acids. The amino acid sequence can consist of amino acid residues 71-80, 71-90, 71-100, 65-80, 60-80, 60-90, or 60-100 of SEQ ID NO:3, or a fragment thereof of at least 6 amino acids. The amino acid sequence need not consist of an amino acid segment of a naturally occurring protein other than Bcl-2. The peptide need not consist of an amino acid segment of a naturally occurring protein other than Bcl-2.

Particular useful are peptides comprising an amino acid sequence having at least 70% identity to the sequence set forth in SEQ ID NO:2, SEQ ID NO:3 or SEQ ID NO:4 that can compete with ATP binding to a Nod-Like Receptor (NLR), or a fragment of the sequence set forth in SEQ ID NO:2, SEQ ID NO:3 or SEQ ID NO:4 of at least 6 amino acids in length that can compete with ATP binding to a NLR. The peptide can inhibit the binding of ATP to the NLR.

Particular useful are peptides comprising an amino acid sequence having at least 70% identity to the sequence set forth in SEQ ID NO:2, SEQ ID NO:3 or SEQ ID NO:4 that can compete with ATP binding to a Nod-Like Receptor (NLR), or a fragment of the sequence set forth in SEQ ID NO:2, SEQ ID NO:3 or SEQ ID NO:4 of at least 8 amino acids in length that can compete with ATP binding to a NLR. The peptide can inhibit the binding of ATP to the NLR.

In some peptides, the amino acid sequence does not consist of an amino acid segment of a naturally occurring protein other than F1L and/or Bcl-2. It is contemplated that such amino acid sequences exclude amino acid segments of naturally occurring proteins (other than F1L and Bcl-2) that happen to contain an amino acid sequence related to the NLR-binding sequence of F1L or Bcl-2. In some peptides, the peptide does not consist of an amino acid segment of a naturally occurring protein other than F1L and/or Bcl-2. It is contemplated that such peptides exclude peptides of naturally occurring proteins (other than F1L and Bcl-2) that happen to contain an amino acid sequence related to the NLR-binding sequence of F1L or Bcl-2. Thus, for example, the amino acid sequence need not consist of an amino acid segment of a naturally occurring protein other than F1L. The peptide need not consist of an amino acid segment of a naturally occurring protein other than F1L. The NLR can be any NLR. For example, the NLR can be NLRP1 or NLRP3. The methionine at position 45 of SEQ ID NO:2 can be oxidized. The methionine at position 45 of SEQ ID NO:2 can be reduced. The peptide can comprise an internalization sequence. The peptide can be conjugated to a tag. The tag can be a label, biotin, streptavidin, avidin, a fluorochrome, a fluorescent label, a label enzyme, a tag, or a combination. The internalization sequence can comprise HIV-Tat protein, Drosophila antennapedia protein, poly-arginine, poly-L-arginine, poly-D-arginine, or a combination. The peptide can be conjugated to a fluorochrome. The peptide can be isolated, purified, and/or recombinant. Also disclosed are compositions comprising any of the disclosed peptides and a pharmaceutically acceptable carrier.

The disclosed peptides can be referred to as binding Nod-like Receptor or binding NLR. There are a variety of different Nod-like Receptors and the disclosed peptides may not bind to all Nod-like Receptors. Thus, for the sake of convenience, reference to a peptide (or other disclosed component) as binding a Nod-like Receptor or binding a NLR is intended to mean that the peptide binds to at least one NLR. If a peptide can bind any of the Nod-like Receptors, the peptide can be referred to as a peptide that “binds NLR.” Such a peptide need only bind a single type of NLR. Of course, if a particular type of NLR is being used and binding of the peptide to this NLR is being used, detected or otherwise assessed, then the peptide must bind the NLR being used. Those of skill in the art will understand such issues form the context of the peptide, NLR and method involved.

Also disclosed is a method of identifying an inhibitor of inflammation. For example, the method can comprise preparing a sample comprising a Nod-Like Receptor (NLR), any of the disclosed peptides, and a candidate agent, detecting the binding of the isolated peptide to the NLR, wherein a decrease in the binding of the isolated peptide and the NLR as compared to a control is an indication that the candidate agent is an inhibitor of inflammation. As another example, the method can comprise preparing a sample comprising a Nod-Like Receptor (NLR), any of the disclosed peptides that can bind to a NLR, and a candidate agent, detecting the binding of the isolated peptide to the NLR, wherein a decrease in the binding of the isolated peptide and the NLR as compared to a control is an indication that the candidate agent is an inhibitor of inflammation.

As another example, the method can comprise preparing a sample comprising a Nod-Like Receptor (NLR), an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and a candidate agent, detecting the binding of the isolated peptide to the NLR, wherein a decrease in the binding of the isolated peptide and the NLR as compared to a control is an indication that the candidate agent is an inhibitor of inflammation. As another example, the method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 8 amino acids in length that can bind to the NLR, and a candidate agent, and detecting the binding of the isolated peptide to the NLR.

A decrease in the binding of the isolated peptide and the NLR as compared to a control is an indication that the candidate agent is an inhibitor of inflammation. Because of the relationship between inflammation, IL-1β production, IL-18 production, IL-33 production, caspase-1 activity, inflammasome activity, inflammasome assembly, NLR activity, and NLR ATP binding, a similar method can be used to identify an inhibitor of IL-1β production, IL-18 production, IL-33 production, caspase-1 activity, inflammasome activity, inflammasome assembly, NLR activity, and NLR ATP binding. In such methods, a decrease in the binding of the isolated peptide and the NLR as compared to a control is an indication that the candidate agent is an inhibitor of IL-1β production, IL-18 production, IL-33 production, caspase-1 activity, inflammasome activity, inflammasome assembly, NLR activity, and NLR ATP binding. The binding of the peptide to the NLR can be measured by fluorescent polarization assay (FPA), time resolved fluorescent resonance energy transfer (TR-FRET), and/or scintillation proximity assay (SPA). The peptide can be conjugated to a fluorochrome, wherein binding of the isolated peptide to the NLR can be measured by fluorescent polarization assay (FPA). The isolated peptide to the NLR can be measured by time resolved fluorescent resonance energy transfer (TR-FRET). Binding of the isolated peptide to the NLR can be measured by scintillation proximity assay (SPA).

Also disclosed is a method of treating inflammation in a subject. For example, the method can comprise administering to the subject any of the disclosed peptides. As another example, the method can comprise administering to the subject any of the disclosed peptides that can bind to a Nod-Like Receptor (NLR). As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR.

As another example, the method can comprise administering to the subject a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. The subject can be a subject identified as having or being at risk for inflammation or an inflammation related disease.

The inflammation can be acute and/or chronic. The inflammation can be caused or exacerbated by IL-1β secretion. The IL-1β secretion can be activated by inflammasome-mediated caspase-1 activation. The inflammation can be caused or exacerbated by Vitiligo.

Also disclosed are methods of treating Bacillus anthracis infection or ameliorating Bacillus anthracis symptoms in a subject. For example, the method can comprise administering to the subject any of the disclosed peptides. As another example, the method can comprise administering to the subject any of the disclosed peptides that can bind to a Nod-Like Receptor (NLR). As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR. As another example, the method can comprise administering to the subject a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. The subject can be a subject identified as having or being at risk for Bacillus anthracis infection.

Also disclosed is a method of inhibiting apoptosis. For example, the method can comprise administering to the subject any of the disclosed peptides. As another example, the method can comprise administering to the subject any of the disclosed peptides that can bind to a Nod-Like Receptor (NLR). As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR. As another example, the method can comprise administering to the subject a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. The subject can be a subject identified as having or being at risk of apoptosis or an apoptosis related disease.

Also disclosed are methods of detecting Nod-Like Receptors (NLRs). For example, the method can comprise (a) bringing into contact a sample and any of the disclosed peptides, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. As another example, the method can comprise (a) bringing into contact a sample and any of the disclosed peptides that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate.

As another example, the method can comprise (a) bringing into contact a sample and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. As another example, the method can comprise (a) bringing into contact a sample and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. The first tag can be, for example, biotin and the second tag can be streptavidin or avidin, or vice versa. The support surface can comprise a bead, a plate, or a multi-well plate. The NLR can be detected via, for example, an enzyme-linked immunosorbent assay.

Also disclosed are methods of identifying inhibitory sites on Nod-Like Receptors (NLRs). For example, the method can comprise bringing into contact a NLR and any of the disclosed peptides, and detecting the location where the peptide binds the NLR. As another example, the method can comprise bringing into contact a NLR and any of the disclosed peptides that can bind to a NLR, and detecting the location where the peptide binds the NLR. As another example, the method can comprise bringing into contact a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and detecting the location where the peptide binds the NLR. As another example, the method can comprise bringing into contact a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, and detecting the location where the peptide binds the NLR. The location where the peptide binds the NLR can be detected using, for example, nuclear magnetic resonance. The location where the peptide binds the NLR can be detected using, for example x-ray crystallography.

Also disclosed are methods of treating a subject suffering from a viral disease. For example, the method can comprise administering to the subject any of the disclosed peptides, wherein the subject is suffering from a viral disease. As another example, the method can comprise administering to the subject any of the disclosed peptides that can compete with F1L, Bcl-2, or both for binding a NLR, wherein the subject is suffering from a viral disease. As another example, the method can comprise administering to the subject an isolated peptide comprising an amino acid sequence having at least 70% identity to the sequence set forth in SEQ ID NO:2, SEQ ID NO:3, or SEQ ID NO:4 that can compete with F1L, Bcl-2, or both for binding a Nod-Like Receptor (NLR) and, or a fragment the sequence set forth in SEQ ID NO:2, SEQ ID NO:3, or SEQ ID NO:4 of at least 6 amino acids in length that can compete with F1L, Bcl-2, or both for binding a NLR, wherein the subject is suffering from a viral disease. As another example, the method can comprise administering to the subject an isolated peptide comprising an amino acid sequence having at least 70% identity to the sequence set forth in SEQ ID NO:2, SEQ ID NO:3, or SEQ ID NO:4 that can compete with F1L, Bcl-2, or both for binding a Nod-Like Receptor (NLR) and, or a fragment the sequence set forth in SEQ ID NO:2, SEQ ID NO:3, or SEQ ID NO:4 of at least 8 amino acids in length that can compete with F1L, Bcl-2, or both for binding a NLR, wherein the subject is suffering from a viral disease. The subject can be a subject identified as having or being at risk of viral disease or a virus-related disease. The subject can be a subject identified as having or being at risk of viral infection.

Also disclosed are methods of identifying compounds that can bind to a Nod-Like Receptor (NLR). For example, the method can comprise modeling the interaction of a NLR and any of the disclosed peptides, selecting or designing compounds predicted to have similar contacts with the NLR as the peptide, and assessing the interaction of the compound using modeling software and/or a modeling system. As another example, the method can comprise modeling the interaction of a NLR and any of the disclosed peptides that can bind to a Nod-Like Receptor (NLR), selecting or designing compounds predicted to have similar contacts with the NLR as the peptide, and assessing the interaction of the compound using modeling software and/or a modeling system. As another example, the method can comprise modeling the interaction of a NLR and any of the disclosed peptides that can compete with F1L, Bcl-2, or both for binding a Nod-Like Receptor (NLR), selecting or designing compounds predicted to have similar contacts with the NLR as the peptide, and assessing the interaction of the compound using modeling software and/or a modeling system. As another example, the method can comprise modeling the interaction of a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, selecting or designing compounds predicted to have similar contacts with the NLR as the peptide, and assessing the interaction of the compound using modeling software and/or a modeling system. As another example, the method can comprise modeling the interaction of a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, selecting or designing compounds predicted to have similar contacts with the NLR as the peptide, and assessing the interaction of the compound using modeling software and/or a modeling system.

### A. COMPOSITIONS

### 1. Peptides

Provided herein are peptides useful for treating inflammation and inflammation related diseases such as inflammatory bowel disease, Crohn's disease, ulcerative colitis, arthritis, psoriasis, Alzheimer's disease, cardiovascular disease, diabetes, and sepsis and for identifying compounds that can be used for treating inflammation and inflammation related diseases such as inflammatory bowel disease, Crohn's disease, ulcerative colitis, arthritis, psoriasis, Alzheimer's disease, cardiovascular disease, diabetes, and sepsis. The peptide can comprise all or a portion of the amino acid sequence SEQ ID NO:2, SEQ ID NO:3 or SEQ ID NO:4 that can bind to a Nod-Like Receptor (NLR). Thus, the peptide can be encoded by, for example, the nucleic acid sequence SEQ ID NO:1 (which is the nucleotide sequence of the F1L open reading frame of vaccinia virus). The ID for the F1L amino acid sequence is YP_{—}232922 and can be found at the web site ncbi.nlm.nih.gov/entrez/viewer.fcgi?val=YP_{—}232922.1. The ID for the Bcl-2 amino acid sequence is NP_{—}000648 (beta isoform) and NP_{—}000624 (alpha isoform) and can be found at the web sites ncbi.nlm.nih.gov/entrez/viewer.fcgi?db=protein&id=72198346 and ncbi.nlm.nih.gov/entrez/viewer.fcgi?db=protein&id=72198189, respectively.

Thus, provided is a purified peptide, comprising an amino acid sequence at least about 70%, 75%, 80%, 85%, 90%, 92%, 92%, 93%, 94%, 95%, 96%, 97%, 98%, 99%, 100% identical to the sequence SEQ ID NO:2, SEQ ID NO:3 or SEQ ID NO:4, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length. The peptide can bind to a NLR. The amino acid sequence can bind to a NLR.

In some aspects, the peptide disclosed herein decreases Nod-like Receptor activity. In some aspects, the peptide disclosed herein decreases ATP binding to Nod-like Receptor. In some aspects, the peptide disclosed herein decreases inflammasome assembly or inflammasome activity. In some aspects, the peptide disclosed herein decreases caspase-1 activity or activation. In some aspects, the peptide disclosed herein decreases IL-1β production. In some aspects, the peptide disclosed herein decreases IL-18 production. In some aspects, the peptide disclosed herein decreases IL-33 production. In some aspects, the peptide disclosed herein decreases inflammation. In some aspects, the peptide disclosed herein decreases inflammatory activity. In some aspects, the peptide disclosed herein decreases inflammatory response. The methionine at position 45 of SEQ ID NO:2 can be oxidized. The methionine at position 45 of SEQ ID NO:2 can be reduced. The peptide can comprise an internalization sequence. The peptide can be conjugated to a tag. The tag can be a label, biotin, streptavidin, avidin, a fluorochrome, a fluorescent label, a label enzyme, a tag, or a combination. The internalization sequence can comprise HIV-Tat protein, Drosophila antennapedia protein, poly-arginine, poly-L-arginine, poly-D-arginine, or a combination. The peptide can be conjugated to a fluorochrome. The peptide can be isolated, purified, and/or recombinant. Also disclosed are compositions comprising any of the disclosed peptides and a pharmaceutically acceptable carrier.

The amino acid sequence can comprise the sequence SEQ ID NO:2, SEQ ID NO:3 or SEQ ID NO:4, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length. The amino acid sequence can comprise at least 8 consecutive residues of SEQ ID NO:2, SEQ ID NO:3 or SEQ ID NO:4.

The provided peptide can comprise the N-terminal amino acid residues of SEQ ID NO:2, SEQ ID NO:3 or SEQ ID NO:4. Thus, provided is a peptide comprising an amino acid sequence at least about 90% identical to, at least about 80% identical to, or at least about 70% identical to amino acids 1 to 48 of SEQ ID NO:2. Thus, provided is a peptide comprising an amino acid sequence identical to, having only conservative substitutions of, at least about 90% identical to, at least about 80% identical to, or at least about 70% identical to, amino acids 1 to 52, 2 to 52, 3 to 52, 4 to 52, 5 to 52, 6 to 52, 7 to 52, 8 to 52, 9 to 52, 10 to 52, 11 to 52, 12 to 52, 13 to 52, 14 to 52, 15 to 52, 16 to 52, 17 to 52, 18 to 52, 19 to 52, 20 to 52, 21 to 52, 22 to 52, 23 to 52, 24 to 52, 25 to 52, 26 to 52, 27 to 52, 28 to 52, 29 to 52, 30 to 52, 31 to 52, or 32 to 52 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 51, 2 to 51, 3 to 51, 4 to 51, 5 to 51, 6 to 51, 7 to 51, 8 to 51, 9 to 51, 10 to 51, 11 to 51, 12 to 51, 13 to 51, 14 to 51, 15 to 51, 16 to 51, 17 to 51, 18 to 51, 19 to 51, 20 to 51, 21 to 51, 22 to 51, 23 to 51, 24 to 51, 25 to 51, 26 to 51, 27 to 51, 28 to 51, 29 to 51, 30 to 51, 31 to 51, or 32 to 51 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 50, 2 to 50, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 26 to 50, 27 to 50, 28 to 50, 29 to 50, 30 to 50, 31 to 50, or 32 to 50 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 49, 2 to 49, 3 to 49, 4 to 49, 5 to 49, 6 to 49, 7 to 49, 8 to 49, 9 to 49, 10 to 49, 11 to 49, 12 to 49, 13 to 49, 14 to 49, 15 to 49, 16 to 49, 17 to 49, 18 to 49, 19 to 49, 20 to 49, 21 to 49, 22 to 49, 23 to 49, 24 to 49, 25 to 49, 26 to 49, 27 to 49, 28 to 49, 29 to 49, 30 to 49, 31 to 49, or 32 to 49 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 48, 2 to 48, 3 to 48, 4 to 48, 5 to 48, 6 to 48, 7 to 48, 8 to 48, 9 to 48, 10 to 48, 11 to 48, 12 to 48, 13 to 48, 14 to 48, 15 to 48, 16 to 48, 17 to 48, 18 to 48, 19 to 48, 20 to 48, 21 to 48, 22 to 48, 23 to 48, 24 to 48, 25 to 48, 26 to 48, 27 to 48, 28 to 48, 29 to 48, 30 to 48, 31 to 48, or 32 to 48 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 47, 2 to 47, 3 to 47, 4 to 47, 5 to 47, 6 to 47, 7 to 47, 8 to 47, 9 to 47, 10 to 47, 11 to 47, 12 to 47, 13 to 47, 14 to 47, 15 to 47, 16 to 47, 17 to 47, 18 to 47, 19 to 47, 20 to 47, 21 to 47, 22 to 47, 23 to 47, 24 to 47, 25 to 47, 26 to 47, 27 to 47, 28 to 47, 29 to 47, 30 to 47, 31 to 47, or 32 to 47 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 46, 2 to 46, 3 to 46, 4 to 46, 5 to 46, 6 to 46, 7 to 46, 8 to 46, 9 to 46, 10 to 46, 11 to 46, 12 to 46, 13 to 46, 14 to 46, 15 to 46, 16 to 46, 17 to 46, 18 to 46, 19 to 46, 20 to 46, 21 to 46, 22 to 46, 23 to 46, 24 to 46, 25 to 46, 26 to 46, 27 to 46, 28 to 46, 29 to 46, 30 to 46, 31 to 46, or 32 to 46 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 45, 2 to 45, 3 to 45, 4 to 45, 5 to 45, 6 to 45, 7 to 45, 8 to 45, 9 to 45, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 26 to 45, 27 to 45, 28 to 45, 29 to 45, 30 to 45, 31 to 45, or 32 to 45 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 44, 2 to 44, 3 to 44, 4 to 44, 5 to 44, 6 to 44, 7 to 44, 8 to 44, 9 to 44, 10 to 44, 11 to 44, 12 to 44, 13 to 44, 14 to 44, 15 to 44, 16 to 44, 17 to 44, 18 to 44, 19 to 44, 20 to 44, 21 to 44, 22 to 44, 23 to 44, 24 to 44, 25 to 44, 26 to 44, 27 to 44, 28 to 44, 29 to 44, 30 to 44, 31 to 44, or 32 to 44 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 43, 2 to 43, 3 to 43, 4 to 43, 5 to 43, 6 to 43, 7 to 43, 8 to 43, 9 to 43, 10 to 43, 11 to 43, 12 to 43, 13 to 43, 14 to 43, 15 to 43, 16 to 43, 17 to 43, 18 to 43, 19 to 43, 20 to 43, 21 to 43, 22 to 43, 23 to 43, 24 to 43, 25 to 43, 26 to 43, 27 to 43, 28 to 43, 29 to 43, 30 to 43, 31 to 43, or 32 to 43 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 42, 2 to 42, 3 to 42, 4 to 42, 5 to 42, 6 to 42, 7 to 42, 8 to 42, 9 to 42, 10 to 42, 11 to 42, 12 to 42, 13 to 42, 14 to 42, 15 to 42, 16 to 42, 17 to 42, 18 to 42, 19 to 42, 20 to 42, 21 to 42, 22 to 42, 23 to 42, 24 to 42, 25 to 42, 26 to 42, 27 to 42, 28 to 42, 29 to 42, 30 to 42, 31 to 42, or 32 to 42 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 41, 2 to 41, 3 to 41, 4 to 41, 5 to 41, 6 to 41, 7 to 41, 8 to 41, 9 to 41, 10 to 41, 11 to 41, 12 to 41, 13 to 41, 14 to 41, 15 to 41, 16 to 41, 17 to 41, 18 to 41, 19 to 41, 20 to 41, 21 to 41, 22 to 41, 23 to 41, 24 to 41, 25 to 41, 26 to 41, 27 to 41, 28 to 41, 29 to 41, 30 to 41, 31 to 41, or 32 to 41 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 40, 2 to 40, 3 to 40, 4 to 40, 5 to 40, 6 to 40, 7 to 40, 8 to 40, 9 to 40, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 26 to 40, 27 to 40, 28 to 40, 29 to 40, 30 to 40, 31 to 40, or 32 to 40 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 39, 2 to 39, 3 to 39, 4 to 39, 5 to 39, 6 to 39, 7 to 39, 8 to 39, 9 to 39, 10 to 39, 11 to 39, 12 to 39, 13 to 39, 14 to 39, 15 to 39, 16 to 39, 17 to 39, 18 to 39, 19 to 39, 20 to 39, 21 to 39, 22 to 39, 23 to 39, 24 to 39, 25 to 39, 26 to 39, 27 to 39, 28 to 39, 29 to 39, 30 to 39, 31 to 39, or 32 to 39 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 38, 2 to 38, 3 to 38, 4 to 38, 5 to 38, 6 to 38, 7 to 38, 8 to 38, 9 to 38, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 26 to 38, 27 to 38, 28 to 38, 29 to 38, 30 to 38, 31 to 38, or 32 to 38 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 37, 2 to 37, 3 to 37, 4 to 37, 5 to 37, 6 to 37, 7 to 37, 8 to 37, 9 to 37, 10 to 37, 11 to 37, 12 to 37, 13 to 37, 14 to 37, 15 to 37, 16 to 37, 17 to 37, 18 to 37, 19 to 37, 20 to 37, 21 to 37, 22 to 37, 23 to 37, 24 to 37, 25 to 37, 26 to 37, 27 to 37, 28 to 37, 29 to 37, 30 to 37, 31 to 37, or 32 to 37 of SEQ ID NO:2.

Thus, provided is a peptide comprising an amino acid sequence identical to, having only conservative substitutions of, at least about 90% identical to, at least about 80% identical to, or at least about 70% identical to, amino acids 1 to 36, 2 to 36, 3 to 36, 4 to 36, 5 to 36, 6 to 36, 7 to 36, 8 to 36, 9 to 36, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 26 to 36, 27 to 36, 28 to 36, 29 to 36, 30 to 36, 31 to 36, or 32 to 36 of SEQ ID NO:2.

Thus, provided is a peptide comprising an amino acid sequence at least about 90% identical to, at least about 80% identical to, or at least about 70% identical to amino acids 35 to 90 of SEQ ID NO:3 or SEQ ID NO:4. Thus, provided is a peptide comprising an amino acid sequence identical to, having only conservative substitutions of, at least about 90% identical to, at least about 80% identical to, or at least about 70% identical to, amino acids 47 to 90, 48 to 90, 49 to 90, 50 to 90, 51 to 90, 52 to 90, 53 to 90, 54 to 90, 55 to 90, 56 to 90, 57 to 90, 58 to 90, 59 to 90, 60 to 90, 61 to 90, 62 to 90, 63 to 90, 64 to 90, 65 to 90, 66 to 90, 67 to 90, 68 to 90, 69 to 90, 70 to 90, 71 to 90, 72 to 90, 73 to 90, 74 to 90, 75 to 90, 76 to 90, 77 to 90, or 78 to 90 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 89, 48 to 89, 49 to 89, 50 to 89, 51 to 89, 52 to 89, 53 to 89, 54 to 89, 55 to 89, 56 to 89, 57 to 89, 58 to 89, 59 to 89, 60 to 89, 61 to 89, 62 to 89, 63 to 89, 64 to 89, 65 to 89, 66 to 89, 67 to 89, 68 to 89, 69 to 89, 70 to 89, 71 to 89, 72 to 89, 73 to 89, 74 to 89, 75 to 89, 76 to 89, 77 to 89, or 78 to 89 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 88, 48 to 88, 49 to 88, 50 to 88, 51 to 88, 52 to 88, 53 to 88, 54 to 88, 55 to 88, 56 to 88, 57 to 88, 58 to 88, 59 to 88, 60 to 88, 61 to 88, 62 to 88, 63 to 88, 64 to 88, 65 to 88, 66 to 88, 67 to 88, 68 to 88, 69 to 88, 70 to 88, 71 to 88, 72 to 88, 73 to 88, 74 to 88, 75 to 88, 76 to 88, 77 to 88, or 78 to 88 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 87, 48 to 87, 49 to 87, 50 to 87, 51 to 87, 52 to 87, 53 to 87, 54 to 87, 55 to 87, 56 to 87, 57 to 87, 58 to 87, 59 to 87, 60 to 87, 61 to 87, 62 to 87, 63 to 87, 64 to 87, 65 to 87, 66 to 87, 67 to 87, 68 to 87, 69 to 87, 70 to 87, 71 to 87, 72 to 87, 73 to 87, 74 to 87, 75 to 87, 76 to 87, 77 to 87, or 78 to 87 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 86, 48 to 86, 49 to 86, 50 to 86, 51 to 86, 52 to 86, 53 to 86, 54 to 86, 55 to 86, 56 to 86, 57 to 86, 58 to 86, 59 to 86, 60 to 86, 61 to 86, 62 to 86, 63 to 86, 64 to 86, 65 to 86, 66 to 86, 67 to 86, 68 to 86, 69 to 86, 70 to 86, 71 to 86, 72 to 86, 73 to 86, 74 to 86, 75 to 86, 76 to 86, 77 to 86, or 78 to 86 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 85, 48 to 85, 49 to 85, 50 to 85, 51 to 85, 52 to 85, 53 to 85, 54 to 85, 55 to 85, 56 to 85, 57 to 85, 58 to 85, 59 to 85, 60 to 85, 61 to 85, 62 to 85, 63 to 85, 64 to 85, 65 to 85, 66 to 85, 67 to 85, 68 to 85, 69 to 85, 70 to 85, 71 to 85, 72 to 85, 73 to 85, 74 to 85, 75 to 85, 76 to 85, 77 to 85, or 78 to 85 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 84, 48 to 84, 49 to 84, 50 to 84, 51 to 84, 52 to 84, 53 to 84, 54 to 84, 55 to 84, 56 to 84, 57 to 84, 58 to 84, 59 to 84, 60 to 84, 61 to 84, 62 to 84, 63 to 84, 64 to 84, 65 to 84, 66 to 84, 67 to 84, 68 to 84, 69 to 84, 70 to 84, 71 to 84, 72 to 84, 73 to 84, 74 to 84, 75 to 84, 76 to 84, 77 to 84, or 78 to 84 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 83, 48 to 83, 49 to 83, 50 to 83, 51 to 83, 52 to 83, 53 to 83, 54 to 83, 55 to 83, 56 to 83, 57 to 83, 58 to 83, 59 to 83, 60 to 83, 61 to 83, 62 to 83, 63 to 83, 64 to 83, 65 to 83, 66 to 83, 67 to 83, 68 to 83, 69 to 83, 70 to 83, 71 to 83, 72 to 83, 73 to 83, 74 to 83, 75 to 83, 76 to 83, 77 to 83, or 78 to 83 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 82, 48 to 82, 49 to 82, 50 to 82, 51 to 82, 52 to 82, 53 to 82, 54 to 82, 55 to 82, 56 to 82, 57 to 82, 58 to 82, 59 to 82, 60 to 82, 61 to 82, 62 to 82, 63 to 82, 64 to 82, 65 to 82, 66 to 82, 67 to 82, 68 to 82, 69 to 82, 70 to 82, 71 to 82, 72 to 82, 73 to 82, 74 to 82, 75 to 82, 76 to 82, 77 to 82, or 78 to 82 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 81, 48 to 81, 49 to 81, 50 to 81, 51 to 81, 52 to 81, 53 to 81, 54 to 81, 55 to 81, 56 to 81, 57 to 81, 58 to 81, 59 to 81, 60 to 81, 61 to 81, 62 to 81, 63 to 81, 64 to 81, 65 to 81, 66 to 81, 67 to 81, 68 to 81, 69 to 81, 70 to 81, 71 to 81, 72 to 81, 73 to 81, 74 to 81, 75 to 81, 76 to 81, or 77 to 81 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 80, 48 to 80, 49 to 80, 50 to 80, 51 to 80, 52 to 80, 53 to 80, 54 to 80, 55 to 80, 56 to 80, 57 to 80, 58 to 80, 59 to 80, 60 to 80, 61 to 80, 62 to 80, 63 to 80, 64 to 80, 65 to 80, 66 to 80, 67 to 80, 68 to 80, 69 to 80, 70 to 80, 71 to 80, 72 to 80, 73 to 80, 74 to 80, 75 to 80, or 76 to 80 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 79, 48 to 79, 49 to 79, 50 to 79, 51 to 79, 52 to 79, 53 to 79, 54 to 79, 55 to 79, 56 to 79, 57 to 79, 58 to 79, 59 to 79, 60 to 79, 61 to 79, 62 to 79, 63 to 79, 64 to 79, 65 to 79, 66 to 79, 67 to 79, 68 to 79, 69 to 79, 70 to 79, 71 to 79, 72 to 79, 73 to 79, 74 to 79, or 75 to 79 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 78, 48 to 78, 49 to 78, 50 to 78, 51 to 78, 52 to 78, 53 to 78, 54 to 78, 55 to 78, 56 to 78, 57 to 78, 58 to 78, 59 to 78, 60 to 78, 61 to 78, 62 to 78, 63 to 78, 64 to 78, 65 to 78, 66 to 78, 67 to 78, 68 to 78, 69 to 78, 70 to 78, 71 to 78, 72 to 78, 73 to 78, or 74 to 78 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 77, 48 to 77, 49 to 77, 50 to 77, 51 to 77, 52 to 77, 53 to 77, 54 to 77, 55 to 77, 56 to 77, 57 to 77, 58 to 77, 59 to 77, 60 to 77, 61 to 77, 62 to 77, 63 to 77, 64 to 77, 65 to 77, 66 to 77, 67 to 77, 68 to 77, 69 to 77, 70 to 77, 71 to 77, 72 to 77, or 73 to 77 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 76, 48 to 76, 49 to 76, 50 to 76, 51 to 76, 52 to 76, 53 to 76, 54 to 76, 55 to 76, 56 to 76, 57 to 76, 58 to 76, 59 to 76, 60 to 76, 61 to 76, 62 to 76, 63 to 76, 64 to 76, 65 to 76, 66 to 76, 67 to 76, 68 to 76, 69 to 76, 70 to 76, 71 to 76, or 72 to 76 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 75, 48 to 75, 49 to 75, 50 to 75, 51 to 75, 52 to 75, 53 to 75, 54 to 75, 55 to 75, 56 to 75, 57 to 75, 58 to 75, 59 to 75, 60 to 75, 61 to 75, 62 to 75, 63 to 75, 64 to 75, 65 to 75, 66 to 75, 67 to 75, 68 to 75, 69 to 75, 70 to 75, or 71 to 75 of SEQ ID NO:3 or SEQ ID NO:4.

Thus, provided is a peptide comprising an amino acid sequence identical to, having only conservative substitutions of, at least about 90% identical to, at least about 80% identical to, or at least about 70% identical to, amino acids 47 to 74, 48 to 74, 49 to 74, 50 to 74, 51 to 74, 52 to 74, 53 to 74, 54 to 74, 55 to 74, 56 to 74, 57 to 74, 58 to 74, 59 to 74, 60 to 74, 61 to 74, 62 to 74, 63 to 74, 64 to 74, 65 to 74, 66 to 74, 67 to 74, 68 to 74, 69 to 74, or 70 to 74 of SEQ ID NO:3 or SEQ ID NO:4.

The amino acid sequence can comprise or consist of amino acids 32-37 of SEQ ID NO:2, amino acids 32-37 of SEQ ID NO:2 where one or both of amino acids 34 and 36 can be substituted independently with any amino acid, or amino acids 71-80 of SEQ ID NO:3 where one or both of amino acids 34 and 36 can be substituted independently with E, A, G, V, L, F, I, W, or P.

The amino acid sequence can comprise or consist of NVDHDY (SEQ ID NO:34); NVXHXY (SEQ ID NO:35), where X at amino acids 34 and 36 can be any amino acid; NVXHXY (SEQ ID NO:36), where X at amino acid 34 can be any amino acid and X at amino acid 36 can be D, E, A, G, V, L, F, I, W, or P; NVXHXY (SEQ ID NO:37), where X at amino acid 34 can be any amino acid and X at amino acid 36 can be D, E, A, G, V, or L; NVXHXY (SEQ ID NO:38), where X at amino acid 34 can be any amino acid and X at amino acid 36 can be D, E, A or G; NVXHXY (SEQ ID NO:39), where X at amino acid 34 can be any amino acid and X at amino acid 36 can be D or E; NVXHDY (SEQ ID NO:40), where X at amino acid 34 can be any amino acid; NVXHAY (SEQ ID NO:41), where X at amino acid 34 can be any amino acid; NVXHXY (SEQ ID NO:42), where X at amino acid 36 can be any amino acid and X at amino acid 34 can be D, E, A, G, V, L, F, I, W, or P; NVXHXY (SEQ ID NO:43), where X at amino acid 36 can be any amino acid and X at amino acid 34 can be D, E, A, G, V, or L; NVXHXY (SEQ ID NO:44), where X at amino acid 36 can be any amino acid and X at amino acid 34 can be D, E, A or G; NVXHXY (SEQ ID NO:45), where X at amino acid 36 can be any amino acid and X at amino acid 34 can be D or E; NVDHXY (SEQ ID NO:46), where X at amino acid 36 can be any amino acid; NVAHXY (SEQ ID NO:47), where X at amino acid 36 can be any amino acid; NVXHXY (SEQ ID NO:48), where X at amino acid 34 can be D, E, A, G, V, L, F, I, W, or P and X at amino acid 36 can be D, E, A, G, V, L, F, I, W, or P; NVXHXY (SEQ ID NO:49), where X at amino acid 34 can be D, E, A, G, V, L, F, I, W, or P and X at amino acid 36 can be D, E, A, G, V, or L; NVXHXY (SEQ ID NO:50), where X at amino acid 34 can be D, E, A, G, V, L, F, I, W, or P and X at amino acid 36 can be D, E, A or G; NVXHXY (SEQ ID NO:51), where X at amino acid 34 can be D, E, A, G, V, L, F, I, W, or P and X at amino acid 36 can be D or E; NVXHDY (SEQ ID NO:52), where X at amino acid 34 can be D, E, A, G, V, L, F, I, W, or P; NVXHAY (SEQ ID NO:53), where X at amino acid 34 can be D, E, A, G, V, L, F, I, W, or P; NVXHXY (SEQ ID NO:54), where X at amino acid 34 can be D, E, A, G, V, or L and X at amino acid 36 can be D, E, A, G, V, L, F, I, W, or P;

NVXHXY (SEQ ID NO:55), where X at amino acid 34 can be D, E, A, G, V, or L and X at amino acid 36 can be D, E, A, G, V, or L; NVXHXY (SEQ ID NO:56), where X at amino acid 34 can be D, E, A, G, V, or L and X at amino acid 36 can be D, E, A, or G; NVXHXY (SEQ ID NO:57), where X at amino acid 34 can be D, E, A, G, V, or L and X at amino acid 36 can be D or E; NVXHDY (SEQ ID NO:58), where X at amino acid 34 can be D, E, A, G, V, or L; NVXHAY (SEQ ID NO:59), where X at amino acid 34 can be D, E, A, G, V, or L; NVXHXY (SEQ ID NO:60), where X at amino acid 34 can be D, E, A or G and X at amino acid 36 can be D, E, A, G, V, L, F, I, W, or P; NVXHXY (SEQ ID NO:61), where X at amino acid 34 can be D, E, A or G and X at amino acid 36 can be D, E, A, G, V, or L; NVXHXY (SEQ ID NO:62), where X at amino acid 34 can be D, E, A or G and X at amino acid 36 can be D, E, A, or G; NVXHXY (SEQ ID NO:63), where X at amino acid 34 can be D, E, A or G and X at amino acid 36 can be D or E; NVXHDY (SEQ ID NO:64), where X at amino acid 34 can be D, E, A or G; NVXHAY (SEQ ID NO:65), where X at amino acid 34 can be D, E, A or G; NVXHXY (SEQ ID NO:66), where X at amino acid 34 can be D or E and X at amino acid 36 can be D, E, A, G, V, L, F, I, W, or P; NVXHXY (SEQ ID NO:67), where X at amino acid 34 can be D or E and X at amino acid 36 can be D, E, A, G, V, or L; NVXHXY (SEQ ID NO:68), where X at amino acid 34 can be D or E and X at amino acid 36 can be D, E, A, or G; NVXHXY (SEQ ID NO:69), where X at amino acid 34 can be D or E and X at amino acid 36 can be D or E; NVXHDY (SEQ ID NO:70), where X at amino acid 34 can be D or E; NVXHAY (SEQ ID NO:71), where X at amino acid 34 can be D or E; NVDHXY (SEQ ID NO:72), where X at amino acid 36 can be D, E, A, G, V, L, F, I, W, or P; NVDHXY (SEQ ID NO:73), where X at amino acid 36 can be D, E, A, G, V, or L; NVDHXY (SEQ ID NO:74), where X at amino acid 36 can be D, E, A, or G; NVDHXY (SEQ ID NO:75), where X at amino acid 36 can be D or E; NVDHAY (SEQ ID NO:76); NVXHXY (SEQ ID NO:77), where X at amino acid 36 can be D, E, A, G, V, L, F, I, W, or P and X at amino acid 34 can be D, E, A, G, V, or L; NVXHXY (SEQ ID NO:78), where X at amino acid 36 can be D, E, A, G, V, L, F, I, W, or P and X at amino acid 34 can be D, E, A or G; NVXHXY (SEQ ID NO:79), where X at amino acid 36 can be D, E, A, G, V, L, F, I, W, or P and X at amino acid 34 can be D or E; NVDHXY (SEQ ID NO:80), where X at amino acid 36 can be D, E, A, G, V, L, F, I, W, or P; NVAHXY (SEQ ID NO:81), where X at amino acid 36 can be D, E, A, G, V, L, F, I, W, or P; NVXHXY (SEQ ID NO:82), where X at amino acid 36 can be D, E, A, G, V, or L and X at amino acid 34 can be D, E, A, G, V, L, F, I, W, or P; NVXHXY (SEQ ID NO:83), where X at amino acid 36 can be D, E, A, G, V, or L and X at amino acid 34 can be D, E, A, G, V, or L; NVXHXY (SEQ ID NO:84), where X at amino acid 36 can be D, E, A, G, V, or L and X at amino acid 34 can be D, E, A, or G; NVXHXY (SEQ ID NO:85), where X at amino acid 36 can be D, E, A, G, V, or L and X at amino acid 34 can be D or E; NVDHXY (SEQ ID NO:86), where X at amino acid 36 can be D, E, A, G, V, or L; NVAHXY (SEQ ID NO:87), where X at amino acid 36 can be D, E, A, G, V, or L; NVXHXY (SEQ ID NO:88), where X at amino acid 36 can be D, E, A or G and X at amino acid 34 can be D, E, A, G, V, L, F, I, W, or P; NVXHXY (SEQ ID NO:89), where X at amino acid 36 can be D, E, A or G and X at amino acid 34 can be D, E, A, G, V, or L; NVXHXY (SEQ ID NO:90), where X at amino acid 36 can be D, E, A or G and X at amino acid 34 can be D, E, A, or G; NVXHXY (SEQ ID NO:91), where X at amino acid 36 can be D, E, A or G and X at amino acid 34 can be D or E; NVDHXY (SEQ ID NO:92), where X at amino acid 36 can be D, E, A or G; NVAHXY (SEQ ID NO:93), where X at amino acid 36 can be D, E, A or G; NVXHXY (SEQ ID NO:94), where X at amino acid 36 can be D or E and X at amino acid 34 can be D, E, A, G, V, L, F, I, W, or P; NVXHXY (SEQ ID NO:95), where X at amino acid 36 can be D or E and X at amino acid 34 can be D, E, A, G, V, or L; NVXHXY (SEQ ID NO:96), where X at amino acid 36 can be D or E and X at amino acid 34 can be D, E, A, or G; NVXHXY (SEQ ID NO:97), where X at amino acid 36 can be D or E and X at amino acid 34 can be D or E; NVDHXY (SEQ ID NO:98), where X at amino acid 36 can be D or E; NVAHXY (SEQ ID NO:99), where X at amino acid 36 can be D or E; NVXHDY (SEQ ID NO:100), where X at amino acid 34 can be D, E, A, G, V, L, F, I, W, or P; NVXHDY (SEQ ID NO:101), where X at amino acid 34 can be D, E, A, G, V, or L; NVXHDY (SEQ ID NO:102), where X at amino acid 34 can be D, E, A, or G; NVXHDY (SEQ ID NO:103), where X at amino acid 34 can be D or E; NVAHDY (SEQ ID NO:104); and NVAHAY (SEQ ID NO:105).

The amino acid sequence can comprise or consist of amino acids 71-80 of SEQ ID NO:3, amino acids 71-80 of SEQ ID NO:3 where one or more of amino acids 72, 73, 76, 77, 78, 79, and 80 can be substituted independently with any amino acid, or amino acids 71-80 of SEQ ID NO:3 where one or more of amino acids 72, 73, 76, 77, 78, 79, and 80 can be substituted independently with A, G, I, V, F, W, or P (for amino acid 72), N, R, A, G, V, L, F, I, W, or P (for amino acid 73), G, V, L, F, I, W, or P (for amino acid 76), G, V, L, F, I, W, or P (for amino acid 77), A, G, V, L, F, I, or W (for amino acid 78), A, V, L, F, I, or W (for amino acid 79), and G, V, L, F, I, W, or P (for amino acid 80).

The amino acid sequence can comprise or consist of PLQTPAAPGA (SEQ ID NO:114); PXXTPXXXXX (SEQ ID NO:115), where X at amino acids 72, 73, and 76-80 can be any amino acid; PXXTPAAXXX (SEQ ID NO:116), where X at amino acids 72, 73, and 78-80 can be any amino acid; PXXTPXXXXX (SEQ ID NO:117), where X at amino acid 72 can be L, A, G, I, V, F, W, or P and X at amino acids 73 and 76-80 can be any amino acid; PXXTPXXXXX (SEQ ID NO:118), where X at amino acid 72 can be L, A, G, I, or V and X at amino acids 73 and 76-80 can be any amino acid; PXXTPXXXXX (SEQ ID NO:119), where X at amino acid 72 can be L, A, or G and X at amino acids 73 and 76-80 can be any amino acid; PLXTPXXXXX (SEQ ID NO:120), where X at amino acids 73 and 76-80 can be any amino acid; PAXTPXXXXX (SEQ ID NO:121), where X at amino acids 73 and 76-80 can be any amino acid; PXXTPAAXXX (SEQ ID NO:122), where X at amino acid 72 can be L, A, G, I, V, F, W, or P and X at amino acids 73 and 76-80 can be any amino acid; PXXTPAAXXX (SEQ ID NO:123), where X at amino acid 72 can be L, A, G, I, or V and X at amino acids 73 and 76-80 can be any amino acid; PXXTPAAXXX (SEQ ID NO:124), where X at amino acid 72 can be L, A, or G and X at amino acids 73 and 76-80 can be any amino acid; PLXTPAAXXX (SEQ ID NO:125), where X at amino acids 73 and 76-80 can be any amino acid; PAXTPAAXXX (SEQ ID NO:126), where X at amino acids 73 and 76-80 can be any amino acid; PXXTPXXXXX (SEQ ID NO:127), where X at amino acid 73 can be Q, N, R, A, G, V, L, F, I, W, or P and X at amino acids 73 and 76-80 can be any amino acid; PXXTPXXXXX (SEQ ID NO:128), where X at amino acid 73 can be Q, N, R, A, G, V, or L and X at amino acids 73 and 76-80 can be any amino acid; PXXTPXXXXX (SEQ ID NO:129), where X at amino acid 73 can be Q, N, R, A, or G and X at amino acids 73 and 76-80 can be any amino acid; PXXTPXXXXX (SEQ ID NO:130), where X at amino acid 73 can be Q, N, or R and X at amino acids 73 and 76-80 can be any amino acid; PXQTPXXXXX (SEQ ID NO:131), where X at amino acids 72 and 76-80 can be any amino acid; PXATPXXXXX (SEQ ID NO:132), where X at amino acids 72 and 76-80 can be any amino acid; PXXTPAAXXX (SEQ ID NO:133), where X at amino acid 73 can be Q, N, R, A, G, V, L, F, I, W, or P and X at amino acids 73 and 76-80 can be any amino acid; PXXTPAAXXX (SEQ ID NO:134), where X at amino acid 73 can be Q, N, R, A, G, V, or L and X at amino acids 73 and 76-80 can be any amino acid; PXXTPAAXXX (SEQ ID NO:135), where X at amino acid 73 can be Q, N, R, A, or G and X at amino acids 73 and 76-80 can be any amino acid; PXXTPAAXXX (SEQ ID NO:136), where X at amino acid 73 can be Q, N, or R and X at amino acids 73 and 76-80 can be any amino acid; PXQTPAAXXX (SEQ ID NO:137), where X at amino acids 72 and 76-80 can be any amino acid; PXATPAAXXX (SEQ ID NO:138), where X at amino acids 72 and 76-80 can be any amino acid; PXXTPXXXXX (SEQ ID NO:139), where X at amino acid 76 can be A, G, V, L, F, I, W, or P and X at amino acids 72, 73, and 77-80 can be any amino acid; PXXTPXXXXX (SEQ ID NO:140), where X at amino acid 76 can be A, G, V, or L and X at amino acids 72, 73, and 77-80 can be any amino acid; PXXTPXXXXX (SEQ ID NO:141), where X at amino acid 76 can be A or G and X at amino acids 72, 73, and 77-80 can be any amino acid; PXXTPAXXXX (SEQ ID NO:142), where X at amino acids 72, 73, and 77-80 can be any amino acid; PXXTPXXXXX (SEQ ID NO:143), where X at amino acid 77 can be A, G, V, L, F, I, W, or P and X at amino acids 72, 73, 76, and 78-80 can be any amino acid; PXXTPXXXXX (SEQ ID NO:144), where X at amino acid 77 can be A, G, V, or L and X at amino acids 72, 73, 76, and 78-80 can be any amino acid; PXXTPXXXXX (SEQ ID NO:145), where X at amino acid 77 can be A or G and X at amino acids 72, 73, 76, and 78-80 can be any amino acid; PXXTPXAXXX (SEQ ID NO:146), where X at amino acids 72, 73, 76, and 78-80 can be any amino acid; PXXTPXXXXX (SEQ ID NO:147), where X at amino acid 78 can be P, A, G, V, L, F, I, or W and X at amino acids 72, 73, 76, 77, 79, and 80 can be any amino acid; PXXTPXXXXX (SEQ ID NO:148), where X at amino acid 78 can be P, A, G, V, or L and X at amino acids 72, 73, 76, 77, 79, and 80 can be any amino acid; PXXTPXXXXX (SEQ ID NO:149), where X at amino acid 78 can be P, A, or G and X at amino acids 72, 73, 76, 77, 79, and 80 can be any amino acid; PXXTPXXPXX (SEQ ID NO:150), where X at amino acids 72, 73, 76, 77, 79, and 80 can be any amino acid; PXXTPXXAXX (SEQ ID NO:151), where X at amino acids 72, 73, 76, 77, 79, and 80 can be any amino acid; PXXTPXXXXX (SEQ ID NO:152), where X at amino acid 79 can be G, A, V, L, F, I, W, or P and X at amino acids 72, 73, 76-78, and 80 can be any amino acid; PXXTPXXXXX (SEQ ID NO:153), where X at amino acid 79 can be G, A, V, or L and X at amino acids 72, 73, 76-78, and 80 can be any amino acid; PXXTPXXXXX (SEQ ID NO:154), where X at amino acid 79 can be G or A and X at amino acids 72, 73, 76-78, and 80 can be any amino acid; PXXTPXXXGX (SEQ ID NO:155), where X at amino acids 72, 73, 76-78, and 80 can be any amino acid; PXXTPXXXAX (SEQ ID NO:156), where X at amino acids 72, 73, 76-78, and 80 can be any amino acid; PXXTPXXXXX (SEQ ID NO:157), where X at amino acid 80 can be A, G, V, L, F, I, W, or P and X at amino acids 72, 73, and 76-79 can be any amino acid; PXXTPXXXXX (SEQ ID NO:158), where X at amino acid 80 can be A, G, V, or L and X at amino acids 72, 73, and 76-79 can be any amino acid; PXXTPXXXXX (SEQ ID NO:159), where X at amino acid 80 can be A, or G and X at amino acids 72, 73, and 76-79 can be any amino acid; and PXXTPXXXXA (SEQ ID NO:160), where X at amino acids 72, 73, and 76-79 can be any amino acid.

a. Fusion Protein

Also provided is a fusion protein comprising the disclosed peptide. Fusion proteins, also know as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6xhis-tag) which can be isolated using nickel or cobalt resins (affinity chromatography) Chimeric proteins can also be manufactured with toxins or anti-bodies attached to them in order to study disease development.

Alternatively, internal ribosome entry sites (IRES) elements can be used to create multigene, or polycistronic, messages. IRES elements are able to bypass the ribosome scanning model of 5′methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781). IRES sequences are known in the art and include those from encephalomycarditis virus (EMCV) (Ghattas, I. R. et al., Mol. Cell. Biol., 11:5848-5849 (1991); BiP protein (Macejak and Sarnow, Nature, 353:91 (1991)); the Antennapedia gene of drosophila (exons d and e) [Oh et al., Genes & Development, 6:1643-1653 (1992)); those in polio virus [Pelletier and Sonenberg, Nature, 334:320325 (1988); see also Mountford and Smith, TIG, 11:179-184 (1985)).

b. Internalization Sequences

The disclosed peptides can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides, polypeptide fragments, or peptides, wherein the linker does not otherwise contribute to the essential function of the composition. A peptide as provided herein can have an amino acid linker comprising, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided peptide can further have deleted N-terminal, C-terminal or intermediate amino acids that do not contribute to the essential activity of the polypeptide.

The disclosed peptide can be linked to an internalization sequence or a protein transduction domain to effectively enter the cell. Several cell penetrating peptides, including the TAT transactivation domain of the HIV virus, antennapedia, and transportan that can readily transport molecules and small peptides across the plasma membrane have been described (Schwarze et al., 1999; Derossi et al., 1996; Yuan et al., 2002). More recently, polyarginine has shown an even greater efficiency of transporting peptides and proteins across the plasma, membrane making it an attractive tool for peptide mediated transport (Fuchs and Raines, 2004). Nonaarginine (R₉, SEQ ID NO:21) has been described as one of the most efficient polyarginine based protein transduction domains, with maximal uptake of significantly greater than TAT or antennapeadia. Peptide mediated cytotoxicity has also been shown to be less with polyarginine-based internalization sequences. R₉ mediated membrane transport is facilitated through heparan sulfate proteoglycan binding and endocytic packaging. Once internalized, heparan is degraded by heparanases, releasing R₉ which leaks into the cytoplasm (Deshayes et al., 2005). Studies have recently shown that derivatives of polyarginine can deliver a full length p53 protein to oral cancer cells, suppressing their growth and metastasis, defining polyarginine as a potent cell penetrating peptide (Takenobu et al., 2002).

Thus, the disclosed peptides can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 3).

TABLE 3
Cell Internalization Transporters

| Name | Sequence | SEQ ID NO |
|---|---|---|
| Polyarginine | RRRRRRRRR | SEQ ID NO: 21 |
| Antp | RQPKIWFPNRRKPWKK | SEQ ID NO: 22 |
| HIV-Tat | GRKKRRQRPPQ | SEQ ID NO: 5 |
| Penetratin | RQIKIWFQNRRMKWKK | SEQ ID NO: 6 |
| Antp-3A | RQIAIWFQNRRMKWAA | SEQ ID NO: 7 |
| Tat | RKKRRQRRR | SEQ ID NO: 8 |
| Buforin II | TRSSRAGLQFPVGRVHRLLRK | SEQ ID NO: 9 |
| Transportan | GWTLNSAGYLLGKINKALAALAKKIL | SEQ ID NO: 10 |
| model amphipathic peptide (MAP) | KLALKLALKALKAALKLA | SEQ ID NO: 11 |
| K-FGF | AAVALLPAVLLALLAP | SEQ ID NO: 12 |
| Ku70 | VPMLK- PMLKE | SEQ ID NO: 13 |
| Prion | MANLGYWLLALFVTMWTDVGLCKKRPKP | SEQ ID NO: 14 |
| pVEC | LLIILRRRIRKQAHAHSK | SEQ ID NO: 15 |
| Pep-1 | KETWWETWWTEWSQPKKKRKV | SEQ ID NO: 16 |
| SynB1 | RGGRLSYSRRRFSTSTGR | SEQ ID NO: 17 |
| Pep-7 | SDLWEMMMVSLACQY | SEQ ID NO: 18 |
| HN-1 | TSPLNIHNGQKL | SEQ ID NO: 19 |
| BGSC (Bis-Guanidinium- Spermidine-Cholesterol) | 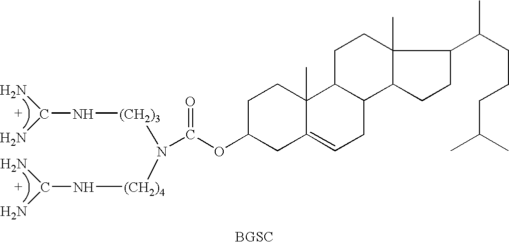 | |
| BGTC (Bis-Guanidinium-Tren- Cholesterol) | 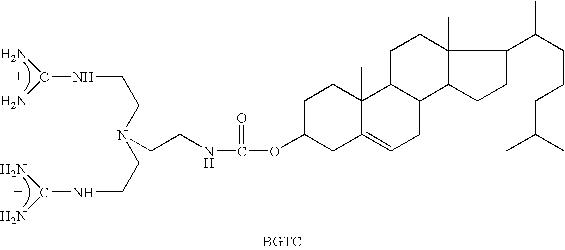 | |

Any other internalization sequences now known or later identified can be combined with the disclosed peptides.

c. Protein Variants

Protein variants and derivatives (which also apply to peptides and to amino acid sequences) are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:2 sets forth a particular sequence of F1L. Specifically disclosed are variants of these and other proteins herein disclosed which have at least, 70% or 75% or 80% or 85% or 90% or 95% homology to the stated sequence. Wherein a sequence is said to have at least about 70% sequence identity, it is understood to also have at least about 75%, 80%, 85%, 90%, 92%, 92%, 93%, 94%, 95%, 96%, 97%, 98%, 99%, or 100% sequence identity.

Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various peptides, proteins and amino acid sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:2 is set forth in SEQ ID NO:1. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH_{I}S—, —CH₂—CH₂—CH═CH—(cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann Rev. Biochem. 61:387 (1992), incorporated herein by reference).

### 2. Nucleic Acids

Also provided is a nucleic acid sequence encoding a polypeptide having the amino acid sequence SEQ ID NO:2, SEQ ID NO:3 or SEQ ID NO:4, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length. Also provided is an isolated nucleic acid comprising a sequence at least about 70% identical to, for example, SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, wherein the sequence encodes a peptide that binds NLR. Thus, for example, the isolated nucleic acid sequence can comprise SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length.

The nucleic acid sequence can comprise a nucleic acid sequence that encodes any of the various peptides disclosed herein.

Also disclosed herein is an expression vector comprising an isolated nucleic acid disclosed herein operably linked to an expression control sequence. Thus, disclosed herein is an expression vector comprising the nucleic acid sequence that encodes any of the various peptides disclosed herein operably linked to an expression control sequence.

The expression control sequence can be a tissue specific promoter. Any tissues specific promoter can be used. For example, neural, tumor, and pancreatic specific promoters are disclosed. Examples of some tissue-specific promoters include but are not limited to MUC1, EIIA, ACTB, WAP, bHLH-EC2, HOXA-1, Alpha-fetoprotein (AFP), opsin, CR1/2, Fc-γ-Receptor 1 (Fc-γ-R1), MMTVD-LTR, the human insulin promoter, Pdha-2. HOXA-1 is a neuronal tissue specific promoter, and as such, proteins expressed under the control of HOXA-1 are only expressed in neuronal tissue. Sequences for these and other tissue-specific promoters are known in the art and can be found, for example, in Genbank, at the web site pubmed.gov.

The expression control sequence can be an inducible promoter. For example, tetracycline controlled transcriptional activation is a method of inducible expression where transcription is reversibly turned on or off in the presence of the antibiotic tetracycline or one of its derivatives (etc. doxycycline). In nature, pTet promotes TetR, the repressor, and TetA, the protein that pumps tetracycline antibiotic out of the cell. Two systems named Tet-off and Tet-on are used.

The Tet-off system makes use of the tetracycline transactivator (tTA) protein created by fusing one protein, TetR (tetracycline repressor), found in Escherichia coli bacteria with another protein, VP16, produced by the Herpes Simplex Virus. The tTA protein binds on DNA at a ‘tet’O operator. Once bound the ‘tet’O operator will activate a promoter coupled to the ‘tet’O operator, activating the transcription of nearby gene. Tetracycline derivatives bind tTA and render it incapable of binding to TRE sequences, therefore preventing transactivation of target genes. This expression system is also used in generation of transgenic mice, which conditionally express gene of interest.

The Tet-on system works in the opposite fashion. In that system the rtTA protein is only capable of binding the operator when bound by doxycycline. Thus the introduction of doxycyline to the system initiates the transcription of the genetic product. The tet-on system is sometimes preferred for the faster responsiveness.

Also disclosed for use in the provided compositions and methods are Cre, FRT and ER (estrogen receptor) conditional gene expression systems. In Cre and FRT systems, activation of knockout of the gene is irreversible once recombination is accomplished, while in Tet and ER systems it is reversible. Tet system has very tight control on expression, while ER system is somewhat leaky. However, Tet system, which depends on transcription and subsequent translation of target gene, is not as fast acting as ER system, which stabilizes the already expressed target protein upon hormone administration.

There are a variety of molecules disclosed herein that are nucleic acid based, including for example the nucleic acids that encode, for example the disclosed peptides, as well as various functional nucleic acids. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to the disclosed peptide and protein molecules, all of which can be encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at the web site ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

iii. Primers and Probes

Disclosed are compositions including primers and probes, which are capable of interacting with the disclosed nucleic acids, such as the nucleic acid encoding the disclosed peptides. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

i. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

ii. Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have an appropriate gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 4. Cells

Also disclosed herein is a cultured cell comprising any of the nucleic acids disclosed herein operably linked to an expression control sequence. The cell can be any cell or cell line, including transformed cells and primary cell lines, that can be used to produce recombinant protein. In some aspects, the cell is a eukaryotic cell. For example, the cell can be a Chinese Hamster Ovary (CHO) cell. CHO cells are a cell line derived from Chinese Hamster ovary cells. The cell can be a HEK 293 cell. HEK29 cells were generated by transformation of human embryonic kidney cell cultures (hence HEK) with sheared adenovirus 5 DNA. The cell can be a SF9 cell. SF9 cells are an insect cell line derived from Spodoptera frugiperda much used for production of recombinant protein.

In some aspects, the cell is a stem cell. One category of stem cells is a pluripotent embryonic stem cell. A “pluripotent stem cell” as used herein means a cell which can give rise to many differentiated cell types in an embryo or adult, including the germ cells (sperm and eggs). Pluripotent stem cells are also capable of self-renewal. Thus, these cells not only populate the germ line and give rise to a plurality of terminally differentiated cells which comprise the adult specialized organs, but also are able to regenerate themselves. One category of stem cells are cells which are capable of self renewal and which can differentiate into cell types of the mesoderm, ectoderm, and endoderm, but which do not give rise to germ cells, sperm or egg.

Another category of stem cells is an adult stem cell which is any type of stem cell that is not derived from an embryo/fetus. For example, recent studies have indicated the presence of a more primitive cell population in the bone marrow capable of self-renewal as well as differentiation into a number of different tissue types other than blood cells. These multi-potential cells were discovered as a minor component in the CD34-plastic-adherent cell population of adult bone marrow, and are variously referred to as mesenchymal stem cells (MSC) (Pittenger, et al., Science 284:143-147 (1999)) or multi-potent adult progenitor cells (MAPC) cells (Furcht, L. T., et al., U.S. patent publication 20040107453 A1). MSC cells do not have a single specific identifying marker, but have been shown to be positive for a number of markers, including CD29, CD90, CD105, and CD73, and negative for other markers, including CD14, CD3, and CD34. Various groups have reported to differentiate MSC cells into myocytes, neurons, pancreatic beta-cells, liver cells, bone cells, and connective tissue. Another group (Wemet et al., U.S. patent publication 20020164794 A1) has described an unrestricted somatic stem cell (USSC) with multi-potential capacity that is derived from a CD45/CD34 population within cord blood. Typically, these stem cells have a limited capacity to generate new cell types and are committed to a particular lineage, although adult stem cells capable of generating all three cell types have been described (for example, United States Patent Application Publication No 20040107453 by Furcht, et al. published Jun. 3, 2004 and PCT/US02/04652, which are both incorporated by reference at least for material related to adult stem cells and culturing adult stem cells). An example of an adult stem cell is the multipotent hematopoietic stem cell, which forms all of the cells of the blood, such as erythrocytes, macrophages, T and B cells. Cells such as these are often referred to as “pluripotent hematopoietic stem cell” for its pluripotency within the hematopoietic lineage. A pluripotent adult stem cell is an adult stem cell having pluripotential capabilities (See for example, United States Patent Publication no. 20040107453, which is U.S. patent application Ser. No. 10/467,963).

Another category of stem cells is a blastocyst-derived stem cell which is a pluripotent stem cell which was derived from a cell which was obtained from a blastocyst prior to the, for example, 64, 100, or 150 cell stage. Blastocyst-derived stem cells can be derived from the inner cell mass of the blastocyst and are the cells commonly used in transgenic mouse work (Evans and Kaufman, (1981) Nature 292:154-156; Martin, (1981) Proc. Natl. Acad. Sci. 78:7634-7638). Blastocyst-derived stem cells isolated from cultured blastocysts can give rise to permanent cell lines that retain their undifferentiated characteristics indefinitely. Blastocyst-derived stem cells can be manipulated using any of the techniques of modern molecular biology, then re-implanted in a new blastocyst. This blastocyst can give rise to a full term animal carrying the genetic constitution of the blastocyst-derived stem cell. (Misra and Duncan, (2002) Endocrine 19:229-238). Such properties and manipulations are generally applicable to blastocyst-derived stem cells. It is understood blastocyst-derived stem cells can be obtained from pre or post implantation embryos and can be referred to as that there can be pre-implantation blastocyst-derived stem cells and post-implantation blastocyst-derived stem cells respectively.

Pluripotential stem cells can be isolated from fetal material, for example, from gonadal tissues, genital ridges, mesenteries or embryonic yolk sacs of embryos or fetal material. For example, such cells can be derived from primordial germ cells (PGCs). Pluripotential stem cells can also be derived from early embryos, such as blastocysts, testes (fetal and adult), and from other pluripotent stem cells such as ES and EG cells following the methods and using the compositions described herein.

The disclosed cells can lack the cell surface molecules required to substantially stimulate allogeneic lymphocytes in a mixed lymphocyte reaction. For example, the cells can lack the surface molecules required to substantially stimulate CD4+ T-cells in in vitro assessments, or in vivo in allogeneic, syngeneic, or autologous recipients. Preferably, the disclosed cells do not cause any substantial adverse immunological consequences for in vivo applications. For example, the therapeutic cell cultures can lack detectable amounts of at least two, or several, or all of the stimulating proteins HLA-DR, HLA-DP, HLA-DQ, CD80, CD86, and B7-H2, as determined by flow cytometry. Those lacking all of the foregoing are most preferred. Also preferred are therapeutic cell cultures which further lack detectable amounts of one or both of the immuno-modulating proteins HLA-G and CD178, as determined by flow cytometry. Also preferred are therapeutic cell cultures which express detectable amounts of the immuno-modulating protein PD-L2, as determined by flow cytometry. In one embodiment, the therapeutic cell culture does not substantially stimulate a lymphocyte mediated response in vitro, as compared to allogeneic controls in a mixed lymphocyte reaction.

### 5. Functional Nucleic Acids

Also disclosed herein are functional nucleic acids. In some aspects, the functional nucleic acid is an agonist of the activity of the disclosed peptides. In some aspects, the functional nucleic acid is an inhibitor of the expression or activity of the disclosed peptides. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, for example, functional nucleic acids can interact with the mRNA of the disclosed peptides or the genomic DNA of the disclosed peptides or they can interact with the disclosed peptides. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293).

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408).

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10-6, 10-8, 10-10, or 10-12. Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Massachusetts), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences encoding the disclosed peptides.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

### 6. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences.

This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 7. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 8. Combination Therapies

Provided herein is a composition that comprises the disclosed peptide and any known or newly discovered substance that can be administered systemically or to the site of a inflammation, cardiovascular disease, cancer, neurodegeneration, or metabolic disregulation. For example, the provided composition can further comprise one or more of classes of antibiotics (e.g. Aminoglycosides, Cephalosporins, Chloramphenicol, Clindamycin, Erythromycins, Fluoroquinolones, Macrolides, Azolides, Metronidazole, Penicillin's, Tetracycline's, Trimethoprim-sulfamethoxazole, Vancomycin), steroids (e.g. Andranes (e.g. Testosterone), Cholestanes (e.g. Cholesterol), Cholic acids (e.g. Cholic acid), Corticosteroids (e.g. Dexamethasone), Estraenes (e.g. Estradiol), Pregnanes (e.g. Progesterone), narcotic and non-narcotic analgesics (e.g. Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g. Alclofenac; Alclometasone Dipropionate; Algestone Acetonide; alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Decanoate; Deflazacort; Delatestryl; Depo-Testosterone; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Mesterolone; Methandrostenolone; Methenolone; Methenolone Acetate; Methylprednisolone Suleptanate; Morniflumate; Nabumetone; Nandrolone; Naproxen; Naproxen Sodium; Naproxol; Nimazone; Olsalazine Sodium; Orgotein; Orpanoxin; Oxandrolane; Oxaprozin; Oxyphenbutazone; Oxymetholone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Stanozolol; Sudoxicam; Sulindac; Suprofen; Talmetacin; Talniflumate; Talosalate; Tebufelone; Tenidap; Tenidap Sodium; Tenoxicam; Tesicam; Tesimide; Testosterone; Testosterone Blends; Tetrydamine; Tiopinac; Tixocortol Pivalate; Tolmetin; Tolmetin Sodium; Triclonide; Triflumidate; Zidometacin; Zomepirac Sodium), or anti-histaminic agents (e.g. Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

Numerous anti-cancer drugs are available for combination with the present method and compositions. The following are lists of anti-cancer (anti-neoplastic) drugs that can be used in conjunction with the presently disclosed DOC1 activity-enhancing or expression-enhancing methods.

Antineoplastic: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RIII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

The disclosed composition can further comprise one or more additional radiosensitizers. Examples of known radiosensitizers include gemcitabine, 5-fluorouracil, pentoxifylline, and vinorelbine. (Zhang et al., 1998; Lawrence et al., 2001; Robinson and Shewach, 2001; Strunz et al., 2002; Collis et al., 2003; Zhang et al., 2004).

The disclosed composition can further comprise Levodopa. The most widely used form of treatment is L-dopa in various forms. L-dopa is transformed into dopamine in the dopaminergic neurons by L-aromatic amino acid decarboxylase (often known by its former name dopa-decarboxylase). However, only 1-5% of L-DOPA enters the dopaminergic neurons. The remaining L-DOPA is often metabolised to dopamine elsewhere, causing a wide variety of side effects. Due to feedback inhibition, L-dopa results in a reduction in the endogenous formation of L-dopa, and so eventually becomes counterproductive.

The disclosed composition can further comprise Carbidopa or Benserazide. Carbidopa or Benserazide are dopa decarboxylase inhibitors. They help to prevent the metabolism of L-dopa before it reaches the dopaminergic neurons and are general given as combination preparations of carbidopa/levodopa (co-careldopa BAN) co-careldopa combined L-dopa and carbidopa in fixed ratios in such branded products of Sinemetand Parcopa and Benserazide/levodopa (co-beneldopa BAN) as Madopar. There are also controlled release versions of Sinemet and Madopar that spread out the effect of the L-dopa. Duodopa is a combination of levodopa and carbidopa, dispersed as a viscous gel. Using a patient-operated portable pump, the drug is continuously delivered via a tube directly into the upper small intestine, where it is rapidly absorbed.

The disclosed composition can further comprise Talcopone. Talcopone inhibits the COMT enzyme, thereby prolonging the effects of L-dopa, and so has been used to complement L-dopa. A similar drug, entacapone, has similar efficacy and has not been shown to cause significant alterations of liver function. Stalevo contains Levodopa, Carbidopa and Entacopone.

The disclosed composition can further comprise the dopamine-agonists bromocriptine (Parlodel), pergolide (Permax), pramipexole (Mirapex), ropinirole (Requip), cabergoline (Cabaser), apomorphine (Apokyn), or lisuride (Revanil). Dopamine agonists initially act by stimulating some of the dopamine receptors.

The disclosed composition can further comprise an MAO-B inhibitor. For example, selegiline (Eldepryl) and rasagiline (Azilect) reduce the symptoms by inhibiting monoamine oxidase-B (MAO-B), which inhibits the breakdown of dopamine secreted by the dopaminergic neurons. By-products of selegiline include amphetamine and methamphetamine, which can cause side effects such as insomnia.

The disclosed composition can further comprise a nucleic acid encoding glutamic acid decarboxylase (GAD), which catalyses the production of a neurotransmitter called GABA. GABA acts as a direct inhibitor on the overactive cells in the STN.

The herein provide composition can further comprise glial-derived neurotrophic factor (GDNF). Via a series of biochemical reactions, GDNF stimulates the formation of L-dopa.

The disclosed composition can further comprise an acetylcholinesterase inhibitor. Acetylcholinesterase inhibitors reduce the rate at which acetylcholine (ACh) is broken down and hence increase the concentration of ACh in the brain (combatting the loss of ACh caused by the death of the cholinergin neurons). Examples currently marketed include donepezil (Aricept, Eisai and Pfizer), galantamine (Razadyne, Ortho-McNeil Neurologics, US) and rivastigmine (Exelon and Exelon Patch, Novartis). Donepezil and galantamine are taken orally. Rivastigmine has oral forms and a once-daily transdermal patch. [118]

The disclosed composition can further comprise memantine (Namenda, Forest Pharmaceuticals, Axura, Merz GMBh, Ebixa, H. Lundbeck, and Akatinol). Memantine is a novel NMDA receptor antagonist, and has been shown to be moderately clinically efficacious.

The disclosed composition can further comprise one or more cells. The cell can be a stem cell. The stem cell can be a pluripotent stem cell. The cell can be a progenitor cell. The cell can be a neural progenitor cell. The cell can be a stem cell capable of differentiating into a neural cell. Thus, the herein provide composition can further comprise stem cells treated with factors to induce differentiation into neural cells. Other such cells known in the art for treating neurodegenerative disease or delivery of compositions to the brain are contemplated herein.

### 9. Antibodies

Also provided herein is an antibody that specifically binds F1L, Bcl-2 or the disclosed peptide. Thus, provided herein is an antibody that specifically binds any of the disclosed peptides.

Thus, also disclosed are immunodetection methods for detecting F1L, Bcl-2 or the disclosed peptide in a sample using the disclosed antibody. For example, the antibody can be used to identify proteins that bind F1L, Bcl-2 or the disclosed peptide. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

i. Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with an antigen of interest, such as the disclosed peptide, F1L, or Bcl-2. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding extracellular fragments of DR3 and TL1A expressed as a fusion protein with human IgG1 or an epitope tag is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing the extracellular domain of TL1A or DR3 as fusion proteins with a signal sequence fragment. The antigen is produced by inserting a gene fragment in-frame between the signal sequence and the mature protein domain of the TL1A or DR3 nucleotide sequence. This results in the display of the foreign proteins on the surface of the virion. This method allows immunization with whole virus, eliminating the need for purification of target antigens.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure.

The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

The term “antibody” as used herein is meant to include intact molecules as well as fragments thereof, such as, for example, Fab and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab')₂, Fab', Fab and the like, including hybrid fragments. Thus, fragments of the antibodies that retain the ability to bind their specific antigens are provided. For example, fragments of antibodies which maintain DR3 or TL1A binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Techniques can also be adapted for the production of single-chain antibodies specific to an antigenic protein of the present disclosure (see e.g., U.S. Pat. No. 4,946,778). In addition, methods can be adapted for the construction of F (ab) expression libraries (see e.g., Huse, et al., 1989 Science 246: 1275-1281) to allow rapid and effective identification of monoclonal F (ab)fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2)fragment produced by pepsin digestion of an antibody molecule; (ii) an Fab fragment generated by reducing the disulfide bridges of an F ((ab′))(2) fragment; (iii) an F (ab)fragment generated by the treatment of the antibody molecule with papain and a reducing agent and (iv) F (v), fragments.

Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding (Bedzyk et al., 1990; Chaudhary et al., 1990). The linker is chosen to permit the heavy chain and light chain to bind together in their proper conformational orientation. See, for example, Huston, J. S., et al., Methods in Enzym. 203:46-121 (1991), which is incorporated herein by reference. These Fvs lack the constant regions (Fc) present in the heavy and light chains of the native antibody.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, a humanized form of a non-human antibody (or a fragment thereof) is a chimeric antibody or fragment (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As used herein, the term “epitope” is meant to include any determinant capable of specific interaction with the anti-DR3 or anti-TL1A antibodies disclosed. Epitopic determinants usually consist of chemically active surface groupings of molecules such as amino acids or sugar side chains and usually have specific three dimensional structural characteristics, as well as specific charge characteristics.

An “epitope tag” denotes a short peptide sequence unrelated to the function of the antibody or molecule that can be used for purification or crosslinking of the molecule with anti-epitope tag antibodies or other reagents.

By “specifically binds” is meant that an antibody recognizes and physically interacts with its cognate antigen (e.g., a DR3 receptor polypeptide or a TL1A poly peptide) and does not significantly recognize and interact with other antigens; such an antibody may be a polyclonal antibody or a monoclonal antibody, which are generated by techniques that are well known in the art.

The antibody can be bound to a substrate or labeled with a detectable moiety or both bound and labeled. The detectable moieties contemplated with the present compositions include fluorescent, enzymatic and radioactive markers.

### 10. Cell Delivery Systems

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Feigner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### 11. Carriers

The disclosed compositions comprising the disclosed peptides or nucleic acids encoding the disclosed peptides can be combined, conjugated or coupled with or to carriers and other compositions to aid administration, delivery or other aspects of the inhibitors and their use. For convenience, such composition will be referred to herein as carriers. Carriers can, for example, be a small molecule, pharmaceutical drug, fatty acid, detectable marker, conjugating tag, nanoparticle, or enzyme.

The disclosed compositions can be used therapeutically in combination with a pharmaceutically acceptable carrier. By “pharmaceutically acceptable” is meant a material that is not biologically or otherwise undesirable, i.e., the material can be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds can be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The carrier molecule can be covalently linked to the disclosed peptides. The carrier molecule can be linked to the amino terminal end of the disclosed peptides. The carrier molecule can be linked to the carboxy terminal end of the disclosed peptides. The carrier molecule can be linked to an amino acid within the disclosed peptides. The disclosed compositions can further comprise a linker connecting the carrier molecule and disclosed inhibitors. The disclosed peptides can also be conjugated to a coating molecule such as bovine serum albumin (BSA) (see Tkachenko et al., (2003) J Am Chem Soc, 125, 4700-4701) that can be used to coat microparticles, nanoparticles of nanoshells with the inhibitors.

Protein crosslinkers that can be used to crosslink the carrier molecule to the inhibitors, such as the disclosed peptides, are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy)ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido/butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl (4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH(Wood's Reagent) (Methyl-p-hydroxybenzimidate hydrochloride, 98%), DMA (Dimethyladipimidate hydrochloride).

i. Nanoparticles, Microparticles, and Microbubbles

The term “nanoparticle” refers to a nanoscale particle with a size that is measured in nanometers, for example, a nanoscopic particle that has at least one dimension of less than about 100 nm Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, silica nanoparticles, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

For example, the disclosed compositions comprising the disclosed peptides or nucleic acids encoding the disclosed peptides can be immobilized on silica nanoparticles (SNPs). SNPs have been widely used for biosensing and catalytic applications owing to their favorable surface area-to-volume ratio, straightforward manufacture and the possibility of attaching fluorescent labels, magnetic nanoparticles (Yang, H. H. et al. 2005) and semiconducting nanocrystals (Lin, Y. W., et al. 2006).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells.

Targeting molecules can be attached to the disclosed compositions and/or carriers. For example, the targeting molecules can be antibodies or fragments thereof, ligands for specific receptors, or other proteins specifically binding to the surface of the cells to be targeted.

ii. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEB S Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the proprotein convertase inhibitors into liposomes. Generally, the fatty acid is a polar lipid. Thus, the fatty acid can be a phospholipid The provided compositions can comprise either natural or synthetic phospholipid. The phospholipids can be selected from phospholipids containing saturated or unsaturated mono or disubstituted fatty acids and combinations thereof. These phospholipids can be dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

iii. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 12. Molecular Modeling

The NLR and peptides can be modeled based on their structures. This information can be used to model and design compounds that interact with NLRs. For example, NLR and the disclosed peptides can be combined in a solution for collecting spectral data for a NLR/peptide complex, NMR data for this complex, or for growing a crystal of this complex.

Structural data describing a crystal can be obtained, for example, by X-ray diffraction. X-ray diffraction data can be collected by a variety of sources, X-ray wavelengths and detectors. In some embodiments, rotating anodes and synchrotron sources (e.g., Advanced Light Source (ALS), Berkeley, Calif.; or Advanced Photon Source (APS), Argonne, Ill.) can be used as the source(s) of X-rays. In some embodiments, area detectors and/or charge-couple devices (CCDs) can be used as the detector(s).

X-ray diffraction data of a crystal of a complex of the NLR bound to a peptide can be used to obtain the structural coordinates of the atoms in the complex. The structural coordinates are Cartesian coordinates that describe the location of atoms in three-dimensional space in relation to other atoms in the complex. The structural coordinates of the complex can be modified by mathematical manipulation, such as by inversion or integer additions or subtractions. As such, structural coordinates are relative coordinates.

The structural coordinates of a complex of the NLR bound to a peptide can be used to derive a representation (e.g., a two dimensional representation or three dimensional representation) of the complex, a fragment of the complex. Such a representation can be useful for a number of applications, including, for example, the visualization, identification and characterization of an active site of the polypeptide.

Various software programs allow for the graphical representation of a set of structural coordinates to obtain a representation of a complex of the NLR bound to the peptide, or a fragment of one of these complexes. In general, such a representation should accurately reflect (relatively and/or absolutely) structural coordinates, or information derived from structural coordinates, such as distances or angles between features. In some embodiments, the representation is a two-dimensional figure, such as a stereoscopic two-dimensional figure. In certain embodiments, the representation is an interactive two-dimensional display, such as an interactive stereoscopic two-dimensional display. An interactive two-dimensional display can be, for example, a computer display that can be rotated to show different faces of a polypeptide, a fragment of a polypeptide, a complex and/or a fragment of a complex. In some embodiments, the representation is a three-dimensional representation. As an example, a three-dimensional model can be a physical model of a molecular structure (e.g., a ball-and-stick model). As another example, a three dimensional representation can be a graphical representation of a molecular structure (e.g., a drawing or a figure presented on a computer display). A two-dimensional graphical representation (e.g., a drawing) can correspond to a three-dimensional representation when the two-dimensional representation reflects three-dimensional information, for example, through the use of perspective, shading, or the obstruction of features more distant from the viewer by features closer to the viewer. In some embodiments, a representation can be modeled at more than one level. As an example, when the three-dimensional representation includes a polypeptide, such as a complex of the NLR bound to a peptide, the polypeptide can be represented at one or more different levels of structure, such as primary (amino acid sequence), secondary (e.g., .alpha.-helices and .beta.-sheets), tertiary (overall fold), and quaternary (oligomerization state) structure. A representation can include different levels of detail. For example, the representation can include the relative locations of secondary structural features of a protein without specifying the positions of atoms. A more detailed representation could, for example, include the positions of atoms.

In some embodiments, a representation can include information in addition to the structural coordinates of the atoms in a complex of the NLR bound to a peptide. For example, a representation can provide information regarding the shape of a solvent accessible surface, the van der Waals radii of the atoms of the model, and the van der Waals radius of a solvent (e.g., water). Other features that can be derived from a representation include, for example, electrostatic potential, the location of voids or pockets within a macromolecular structure, and the location of hydrogen bonds and salt bridges.

An agent that interacts with a NLR can be identified or designed by a method that includes using a representation of the NLR or a fragment thereof, or a complex of NLR bound to a peptide or a fragment of either one of these complexes. Exemplary types of representations include the representations discussed above. In some embodiments, the representation can be of an analog polypeptide, polypeptide fragment, complex or fragment of a complex. A candidate agent that interacts with the representation can be designed or identified by performing computer fitting analysis of the candidate agent with the representation. In general, an agent is a molecule. Examples of agents include polypeptides, nucleic acids (including DNA or RNA), steroids and non-steroidal organic compounds. An agent that interacts with a polypeptide can interact transiently or stably with the polypeptide. The interaction can be mediated by any of the forces noted herein, including, for example, hydrogen bonding, electrostatic forces, hydrophobic interactions, and van der Waals interactions.

As noted above, X-ray crystallography can be used to obtain structural coordinates of a complex of NLR bound to a peptide. However, such structural coordinates can be obtained using other techniques including NMR techniques. Additional structural information can be obtained from spectral techniques (e.g., optical rotary dispersion (ORD), circular dichroism (CD)), homology modeling, and computational methods (e.g., computational methods that can include data from molecular mechanics, computational methods that include data from dynamics assays).

In some embodiments, the X-ray diffraction data can be used to construct an electron density map of a complex of NLR bound to a peptide or a fragment thereof, and the electron density map can be used to derive a representation (e.g., a two dimensional representation, a three dimensional representation). Creation of an electron density map typically involves using information regarding the phase of the X-ray scatter. Phase information can be extracted, for example, either from the diffraction data or from supplementing diffraction experiments to complete the construction of the electron density map. Methods for calculating phase from X-ray diffraction data include, for example, multiwavelength anomalous dispersion (MAD), multiple isomorphous replacement (MIR), multiple isomorphous replacement with anomalous scattering (MIRAS), single isomorphous replacement with anomalous scattering (SIRAS), reciprocal space solvent flattening, molecular replacement, or any combination thereof. Upon determination of the phase, an electron density map can be constructed. The electron density map can be used to derive a representation of the complex or a fragment thereof by aligning a three-dimensional model of a previously known polypeptide or a previously known complex (e.g., a complex containing a polypeptide bound to a ligand) with the electron density map. For example, the electron density map corresponding to a NLR/peptide complex can be aligned with the electron density map corresponding to NLR complexed to another peptide or compound, such as an agonist or antagonist.

The alignment process results in a comparative model that shows the degree to which the calculated electron density map varies from the model of the previously known polypeptide or the previously known complex. The comparative model is then refined over one or more cycles (e.g., two cycles, three cycles, four cycles, five cycles, six cycles, seven cycles, eight cycles, nine cycles, 10 cycles) to generate a better fit with the electron density map. A software program such as CNS (Brunger et al., Acta Crystallogr. D54:905-921, 1998) can be used to refine the model. The quality of fit in the comparative model can be measured by, for example, an R_{work} or R_{free} value. A smaller value of R_{work} or R_{free} generally indicates a better fit. Misalignments in the comparative model can be adjusted to provide a modified comparative model and a lower R_{work} or R_{free} value. The adjustments can be based on information (e.g., sequence information) relating to NRL or the peptide, the previously known polypeptide and/or the previously known complex. As an example, in embodiments in which a model of a previously known complex of a polypeptide bound to a ligand is used, an adjustment can include replacing the ligand in the previously known complex with the peptide. As another example, in certain embodiments, an adjustment can include replacing an amino acid in the previously known polypeptide with the amino acid in the corresponding site of NRL. When adjustments to the modified comparative model satisfy a best fit to the electron density map, the resulting model is that which is determined to describe the polypeptide or complex from which the X-ray data was derived (e.g., the NRL/peptide complex). Methods of such processes are disclosed, for example, in Carter and Sweet, eds., “Macromolecular Crystallography” in Methods in Enzymology, Vol. 277, Part B, New York: Academic Press, 1997, and articles therein, e.g., Jones and Kjeldgaard, “Electron-Density Map Interpretation,” p. 173, and Kleywegt and Jones, “Model Building and Refinement Practice,” p. 208.

Discussed above is a method of deriving a representation of a complex by aligning a three-dimensional model of a previously known polypeptide or a previously known complex with a newly calculated electron density map corresponding to a crystal of the complex. One adjustment that can be used in this modeling process can include replacing the compound in the representation of the previously known complex with the peptide.

A machine, such as a computer, can be programmed in memory with the structural coordinates of a complex of the NRL bound to the peptide, together with a program capable of generating a graphical representation of the structural coordinates on a display connected to the machine. Alternatively or additionally, a software system can be designed and/or utilized to accept and store the structural coordinates. The software system can be capable of generating a graphical representation of the structural coordinates. The software system can also be capable of accessing external databases to identify compounds (e.g., polypeptides) with similar structural features as NRL, and/or to identify one or more candidate agents with characteristics that may render the candidate agent(s) likely to interact with NRL.

A machine having a memory containing structure data or a software system containing such data can aid in the rational design or selection of NRL agonists and/or NRL antagonists. For example, such a machine or software system can aid in the evaluation of the ability of an agent to associate with a complex of the NRL bound to the peptide, or can aid in the modeling of compounds or proteins related by structural or sequence homology to an NRL. As used herein, an agonist refers to a compound that mimics or enhances at least one activity of NRL, and an antagonist refers to a compound that inhibits at least one activity, or has an opposite activity, of NRL. It is possible that one compound can act as an agonist in one respect and an antagonist in another respect, or that one compound can act as an agonist or antagonist in one respect and can have no effect (neither a positive nor negative effect) in another respect.

The machine can produce a representation (e.g., a two dimensional representation, a three dimensional representation) of a complex of the NRL bound to the peptide or a fragment thereof. A software system, for example, can cause the machine to produce such information. The machine can include a machine-readable data storage medium including a data storage material encoded with machine-readable data. The machine-readable data can include structural coordinates of atoms of a complex of the NRL bound to the peptide, or a fragment thereof. Machine-readable storage media (e.g., data storage material) include, for example, conventional computer hard drives, floppy disks, DAT tape, CD-ROM, DVD, and other magnetic, magneto-optical, optical, and other media which may be adapted for use with a machine (e.g., a computer). The machine can also have a working memory for storing instructions for processing the machine-readable data, as well as a central processing unit (CPU) coupled to the working memory and to the machine-readable data storage medium for the purpose of processing the machine-readable data into the desired three-dimensional representation. A display can be connected to the CPU so that the three-dimensional representation can be visualized by the user. Accordingly, when used with a machine programmed with instructions for using the data (e.g., a computer loaded with one or more programs of the sort described herein) the machine is capable of displaying a graphical representation (e.g., a two dimensional graphical representation, a three-dimensional graphical representation) of any of the polypeptides, polypeptide fragments, complexes, or complex fragments described herein.

A display (e.g., a computer display) can show a representation of a complex of NRL bound to the peptide, or a fragment of either of these complexes. The user can inspect the representation and, using information gained from the representation, generate a model of a complex or fragment thereof that includes an agent other than the peptide. The model can be generated, for example, by altering a previously existing representation of a NRL/peptide complex. Optionally, the user can superimpose a three-dimensional model of an agent on the representation of human NRL bound to the peptide. The agent can be an agonist (e.g., a candidate agonist) of NRL or an antagonist (e.g., a candidate antagonist) of NRL. In some embodiments, the agent can be a known compound or fragment of a compound. In certain embodiments, the agent can be a previously unknown compound, or a fragment of a previously unknown compound.

It can be desirable for the agent to have a shape that complements the shape of the active site. There can be a preferred distance, or range of distances, between atoms of the agent and atoms of the NRL. Distances longer than a preferred distance may be associated with a weak interaction between the agent and active site. Distances shorter than a preferred distance may be associated with repulsive forces that can weaken the interaction between the agent and the polypeptide. A steric clash can occur when distances between atoms are too short. A steric clash occurs when the locations of two atoms are unreasonably close together, for example, when two atoms are separated by a distance less than the sum of their van der Waals radii. If a steric clash exists, the user can adjust the position of the agent relative to the NRL (e.g., a rigid body translation or rotation of the agent), until the steric clash is relieved. The user can adjust the conformation of the agent or of the NRL in the vicinity of the agent in order to relieve a steric clash. Steric clashes can also be removed by altering the structure of the agent, for example, by changing a “bulky group,” such as an aromatic ring, to a smaller group, such as to a methyl or hydroxyl group, or by changing a rigid group to a flexible group that can accommodate a conformation that does not produce a steric clash. Electrostatic forces can also influence an interaction between an agent and a ligand-binding domain. For example, electrostatic properties can be associated with repulsive forces that can weaken the interaction between the agent and the NRL. Electrostatic repulsion can be relieved by altering the charge of the agent, e.g., by replacing a positively charged group with a neutral group.

Forces that influence binding strength between the peptide and NRL can be evaluated in the polypeptide/agent model. These can include, for example, hydrogen bonding, electrostatic forces, hydrophobic interactions, van der Waals interactions, dipole-dipole interactions, π-stacking forces, and cation-π interactions. The user can evaluate these forces visually, for example by noting a hydrogen bond donor/acceptor pair arranged with a distance and angle suitable for a hydrogen bond. Based on the evaluation, the user can alter the model to find a more favorable interaction between the NRL and the agent. Altering the model can include changing the three-dimensional structure of the polypeptide without altering its chemical structure, for example by altering the conformation of amino acid side chains or backbone dihedral angles. Altering the model can include altering the position or conformation of the agent, as described above. Altering the model can also include altering the chemical structure of the agent, for example by substituting, adding, or removing groups. For example, if a hydrogen bond donor on the NRL is located near a hydrogen bond donor on the agent, the user can replace the hydrogen bond donor on the agent with a hydrogen bond acceptor.

The relative locations of an agent and the NRL, or their conformations, can be adjusted to find an optimized binding geometry for a particular agent to the NRL. An optimized binding geometry is characterized by, for example, favorable hydrogen bond distances and angles, maximal electrostatic attractions, minimal electrostatic repulsions, the sequestration of hydrophobic moieties away from an aqueous environment, and the absence of steric clashes. The optimized geometry can have the lowest calculated energy of a family of possible geometries for an NRL/agent complex. An optimized geometry can be determined, for example, through molecular mechanics or molecular dynamics calculations.

A series of representations of complexes of NRL bound to theh peptide, having different bound agents can be generated. A score can be calculated for each representation. The score can describe, for example, an expected strength of interaction between NRL and the agent. The score can reflect one of the factors described above that influence binding strength. The score can be an aggregate score that reflects more than one of the factors. The different agents can be ranked according to their scores.

Steps in the design of the agent can be carried out in an automated fashion by a machine. For example, a representation of NRL can be programmed in the machine, along with representations of candidate agents. The machine can find an optimized binding geometry for each of the candidate agents to the active site, and calculate a score to determine which of the agents in the series is likely to interact most strongly with the NRL.

A software system can be designed and/or implemented to facilitate these steps. Software systems (e.g., computer programs) used to generate representations or perform the fitting analyses include, for example: MCSS, Ludi, QUANTA, Insight II, Cerius2, CHARMm, and Modeler from Accelrys, Inc. (San Diego, Calif.); SYBYL, Unity, FleXX, and LEAPFROG from TRIPOS, Inc. (St. Louis, Mo.); AUTODOCK (Scripps Research Institute, La Jolla, Calif.); GRID (Oxford University, Oxford, UK); DOCK (University of California, San Francisco, Calif.); and Flo+ and Flo99 (Thistlesoft, Morris Township, N.J.). Other useful programs include ROCS, ZAP, FRED, Vida, and Szybki from Openeye Scientific Software (Santa Fe, N. Mex.); Maestro, Macromodel, and Glide from Schrodinger, LLC (Portland, Oreg.); MOE (Chemical Computing Group, Montreal, Quebec), Allegrow (Boston De Novo, Boston, Mass.), and GOLD (Jones et al., J. Mol. Biol. 245:43-53, 1995). The structural coordinates can also be used to visualize the three-dimensional structure of an ERalpha polypeptide using MOLSCRIPT, RASTER3D, or PyMOL (Kraulis, J. Appl. Crystallogr. 24: 946-950, 1991; Bacon and Anderson, J. Mol. Graph. 6: 219-220, 1998; DeLano, The PyMOL Molecular Graphics System (2002) DeLano Scientific, San Carlos, Calif.).

The agent can, for example, be selected by screening an appropriate database, can be designed de novo by analyzing the steric configurations and charge potentials of unbound NRL in conjunction with the appropriate software systems, and/or can be designed using characteristics of known ligands of progesterone receptors or other hormone receptors. The method can be used to design or select agonists or antagonists of NRLs. A software system can be designed and/or implemented to facilitate database searching, and/or agent selection and design.

Once an agent has been designed or identified, it can be obtained or synthesized and further evaluated for its effect on NRL activity. For example, the agent can be evaluated by contacting it with NRL and measuring the effect of the agent on polypeptide activity. A method for evaluating the agent can include an activity assay performed in vitro or in vivo. An activity assay can be a cell-based assay, for example. Depending upon the action of the agent on NRL, the agent can act either as an agonist or antagonist of NRL activity. A crystal containing the NRL bound to the identified agent can be grown and the structure determined by X-ray crystallography. A second agent can be designed or identified based on the interaction of the first agent with NRL.

Various molecular analysis and rational drug design techniques are further disclosed in, for example, U.S. Pat. Nos. 5,834,228, 5,939,528 and 5,856,116, as well as in PCT Application No. PCT/US98/16879, published as WO 99/09148.

### B. METHODS

### 1. Modulating Nod-Like Receptor Activity

Provided herein are methods of inhibiting or enhancing Nod-like Receptor activity in a cell. The method can comprise contacting the cell with a composition comprising the disclosed peptide. Also provided herein is a method of identifying an inhibitor of Nod-like Receptor activity. The method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 8 amino acids in length that can bind to the NLR, and a candidate agent, and detecting the binding of the isolated peptide to the NLR. A decrease in the binding of the peptide and the NLR as compared to a control is an indication that the candidate agent is an inhibitor of Nod-like Receptor activity.

Also provided herein is a method of identifying an inhibitor of inflammation. The method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 6 amino acids in length that can bind to the NLR, and a candidate agent, and detecting the binding of the isolated peptide to the NLR. The method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 8 amino acids in length that can bind to the NLR, and a candidate agent, and detecting the binding of the isolated peptide to the NLR. A decrease in the binding of the peptide and the NLR as compared to a control is an indication that the candidate agent is an inhibitor of inflammation. Because of the relationship between inflammation, IL-1β production, IL-18 production, IL-33 production, caspase-1 activity, inflammasome activity, inflammasome assembly, NLR activity, and NLR ATP binding, a similar method can be used to identify an inhibitor of IL-1β production, IL-18 production, IL-33 production, caspase-1 activity, inflammasome activity, inflammasome assembly, NLR activity, and NLR ATP binding. In such methods, a decrease in the binding of the isolated peptide and the NLR as compared to a control is an indication that the candidate agent is an inhibitor of IL-1β production, IL-18 production, IL-33 production, caspase-1 activity, inflammasome activity, inflammasome assembly, NLR activity, and NLR ATP binding.

The binding of the peptide to the NLR can be measured by fluorescent polarization assay (FPA), time resolved fluorescent resonance energy transfer (TR-FRET), and/or scintillation proximity assay (SPA).

Also disclosed is a method of inhibiting Nod-like Receptor activity in a cell, comprising contacting the cell with a composition comprising the disclosed peptide or a nucleic acid encoding the disclosed peptide. Thus, provided herein is a method of inhibiting Nod-like Receptor activity in a cell, comprising contacting the cell with an isolated nucleic acid comprising a sequence at least about 70%, 75%, 80%, 85%, 90%, 92%, 92%, 93%, 94%, 95%, 96%, 97%, 98%, 99%, 100% identical to SEQ ID NO:1, or a fragment thereof at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 residues in length, operably linked to an expression control sequence, wherein the sequence encodes a peptide that binds NLR.

Also provided is a method of inhibiting Nod-like Receptor activity in a cell, comprising contacting the cell with a purified polypeptide comprising an amino acid sequence at least at least about 70%, 75%, 80%, 85%, 90%, 92%, 92%, 93%, 94%, 95%, 96%, 97%, 98%, 99%, 100% identical to the sequence SEQ ID NO:2, SEQ ID NO:4, or SEQ ID NO:4, or a fragment thereof at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 85, 90, or 100 residues in length, wherein the peptide binds NLR.

### 2. Treatment

Provided herein are compositions and methods for treating a condition or disease affected by Nod-like Receptor levels or activity. The method can comprise administering the disclosed peptide or a nucleic acid encoding the disclosed peptide.

Also disclosed is a method of treating inflammation in a subject. The method can comprise administering to the subject the disclosed peptides. Thus, for example, the method can comprise administering a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. As another example, the method can comprise administering to the subject any of the disclosed peptides that can bind to a Nod-Like Receptor (NLR). As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR. The subject can be a subject identified as having or being at risk for inflammation or an inflammation related disease.

The inflammation can be acute and/or chronic. The inflammation can be caused or exacerbated by IL-1β secretion. The IL-1β secretion can be activated by inflammasome-mediated caspase-1 activation. The inflammation can be caused or exacerbated by Vitiligo.

Also disclosed is a method of treating Bacillus anthracis infection or ameliorating Bacillus anthracis symptoms in a subject. The method can comprise administering to the subject the disclosed peptide. Thus, for example, the method can comprise administering to the subject a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR. The subject can be a subject identified as having or being at risk for Bacillus anthracis infection.

Also disclosed is a method of inhibiting apoptosis. The method can comprise administering to the subject the disclosed peptide. Thus, for example, the method can comprise administering to the subject a peptide comprising an amino acid sequence having at least 70% identity to the sequence set forth in SEQ ID NO:2, SEQ ID NO:3 or SEQ ID NO:4 that can bind to a Nod-Like Receptor (NLR), or a fragment thereof of at least 8 amino acids in length that can bind to a NLR. As another example, the method can comprise administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR. The subject can be a subject identified as having or being at risk apoptosis or an apoptosis related disease.

Thus, provided is a method of treating a subject with a condition or disease affected by Nod-like Receptor levels or activity, the method comprising administering to the subject an expression vector encoding the disclosed peptide, such that a therapeutically effective amount of the peptide is expressed in the subject.

Also provided is a method of treating a subject with a condition or disease affected by Nod-like Receptor levels or activity, the method comprising administering to the subject a cell comprising an expression vector encoding the disclosed peptide, such that a therapeutically effective amount of the peptide is expressed by the cell in the subject. In some aspects, the cell of the methods is a stem cell.

Also provided is a method of treating a subject with a condition or disease affected by Nod-like Receptor levels or activity, the method comprising administering to the subject a therapeutically effective amount of a composition comprising the disclosed peptide.

Also disclosed are methods of treating a subject suffering from a viral disease. For example, the method can comprise administering to the subject any of the disclosed peptides, wherein the subject is suffering from a viral disease. As another example, the method can comprise administering to the subject any of the disclosed peptides that can compete with F1L, Bcl-2, or both for binding a NLR, wherein the subject is suffering from a viral disease. As another example, the method can comprise administering to the subject an isolated peptide comprising an amino acid sequence having at least 70% identity to the sequence set forth in SEQ ID NO:2, SEQ ID NO:3, or SEQ ID NO:4 that can compete with F1L, Bcl-2, or both for binding a Nod-Like Receptor (NLR) and, or a fragment the sequence set forth in SEQ ID NO:2, SEQ ID NO:3, or SEQ ID NO:4 of at least 6 amino acids in length that can compete with F1L, Bcl-2, or both for binding a NLR, wherein the subject is suffering from a viral disease. As another example, the method can comprise administering to the subject an isolated peptide comprising an amino acid sequence having at least 70% identity to the sequence set forth in SEQ ID NO:2, SEQ ID NO:3, or SEQ ID NO:4 that can compete with F1L, Bcl-2, or both for binding a Nod-Like Receptor (NLR) and, or a fragment the sequence set forth in SEQ ID NO:2, SEQ ID NO:3, or SEQ ID NO:4 of at least 8 amino acids in length that can compete with F1L, Bcl-2, or both for binding a NLR, wherein the subject is suffering from a viral disease. The subject can be a subject identified as having or being at risk of viral disease or a virus-related disease. The subject can be a subject identified as having or being at risk of viral infection.

i. Inflammation

Examples of inflammatory diseases include, but are not limited to, chronic inflammatory diseases and acute inflammatory diseases.

According to some aspects, the disclosed peptides can be used to treat chronic inflammatory disease, such as colitis. According to some aspects, the disclosed peptides can be used to treat acute inflammatory disease, such as asthma.

According to some aspects, the disclosed peptides can be used to treat an inflammation associated with hypersensitivity. Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH. According to some aspects of the disclosed peptides, the disclosed peptides can be used to treat Type I or immediate hypersensitivity, such as asthma.

Examples of Type II hypersensitivity include, but are not limited to, rheumatoid diseases, rheumatoid autoimmune diseases, rheumatoid arthritis (Krenn V. et al., Histol Histopathol July 2000; 15 (3): 791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2): 49), sclerosis, systemic sclerosis (Renaudineau Y. et al., Clin Diagn Lab Immunol. March 1999; 6 (2): 156); Chan 0 T. et al., Immunol Rev June 1999; 169: 107), glandular diseases, glandular autoimmune diseases, pancreatic autoimmune diseases, diabetes, Type I diabetes (Zimmet P. Diabetes Res Clin Pract October 1996; 34 Suppl:S125), thyroid diseases, autoimmune thyroid diseases, Graves' disease (Orgiazzi J. Endocrinol Metab Clin North Am June 2000; 29 (2): 339), thyroiditis, spontaneous autoimmune thyroiditis (Braley-Mullen H. and Yu S, J Immunol Dec. 15, 2000; 165 (12): 7262), Hashimoto's thyroiditis (Toyoda N. et al., Nippon Rinsho August 1999; 57 (8): 1810), myxedema, idiopathic myxedema (Mitsuma T. Nippon Rinsho. August 1999; 57 (8): 1759); autoimmune reproductive diseases, ovarian diseases, ovarian autoimmunity (Garza K M. et al., J Reprod Immunol February 1998; 37 (2): 87), autoimmune anti-sperm infertility (Dickman A B. et al., Am J Reprod Immunol. March 2000; 43 (3): 134), repeated fetal loss (Tincani A. et al., Lupus 1998; 7 Suppl 2: S107-9), neurodegenerative diseases, neurological diseases, neurological autoimmune diseases, multiple sclerosis (Cross A H. et al., J Neuroimmunol Jan. 1, 2001; 112 (1-2): 1), Alzheimer's disease (Oron L. et al., J Neural Transm Suppl. 1997; 49: 77), myasthenia gravis (Infante A J. And Kraig E, Int Rev Immunol 1999; 18 (1-2): 83), motor neuropathies (Kornberg A J. J Clin Neurosci. May 2000; 7 (3): 191), Guillain-Barre syndrome, neuropathies and autoimmune neuropathies (Kusunoki S. Am J Med. Sci. April 2000; 319 (4): 234), myasthenic diseases, Lambert-Eaton myasthenic syndrome (Takamori M. Am J Med. Sci. April 2000; 319 (4): 204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) January 2000; 156 (1): 23); neuropathies, dysimmune neuropathies (Nobile-Orazio E. et al., Electroencephalogr Clin Neurophysiol Suppl 1999; 50: 419); neuromyotonia, acquired neuromyotonia, arthrogryposis multiplex congenita (Vincent A. et al., Ann N Y Acad. Sci. May 13, 1998; 841: 482), cardiovascular diseases, cardiovascular autoimmune diseases, atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), granulomatosis, Wegener's granulomatosis, arteritis, Takayasu's arteritis and Kawasaki syndrome (Praprotnik S. et al., Wien Klin Wochenschr Aug. 25, 2000; 112 (15-16): 660); anti-factor VIII autoimmune disease (Lacroix-Desmazes S. et al., Semin Thromb Hemost. 2000; 26 (2): 157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). May 2000; 151 (3): 178); antiphospholipid syndrome (Flamholz R. et al., J Clin Apheresis 1999; 14 (4): 171); heart failure, agonist-like beta-adrenoceptor antibodies in heart failure (Wallukat G. et al., Am J. Cardiol. Jun. 17, 1999; 83 (12A): 75H), thrombocytopenic purpura (Moccia F. Ann Ital Med. Int. April-June 1999; 14 (2): 114); hemolytic anemia, autoimmune hemolytic anemia (Efremov D G. et al., Leuk Lymphoma January 1998; 28 (3-4): 285), gastrointestinal diseases, autoimmune diseases of the gastrointestinal tract, intestinal diseases, chronic inflammatory intestinal disease (Garcia Herola A. et al., Gastroenterol Hepatol. January 2000; 23 (1): 16), celiac disease (Landau Y E. and Shoenfeld Y. Harefuah Jan. 16, 2000; 138 (2): 122), autoimmune diseases of the musculature, myositis, autoimmune myositis, Sjogren's syndrome (Feist E. et al., Int Arch Allergy Immunol September 2000; 123 (1): 92); smooth muscle autoimmune disease (Zauli D. et al., Biomed Pharmacother June 1999; 53 (5-6): 234), hepatic diseases, hepatic autoimmune diseases, autoimmune hepatitis (Manns M P. J Hepatol August 2000; 33 (2): 326) and primary biliary cirrhosis (Strassburg C P. et al., Eur J Gastroenterol Hepatol. June 1999; 11 (6): 595).

According to some aspects, the disclosed method can be used to treat Type IV or T lymphocyte mediated hypersensitivity. For example, the disclosed method can be used to treat Type IV or T lymphocyte mediated hypersensitivity such as DTH.

Examples of Type IV or T cell mediated hypersensitivity, include, but are not limited to, rheumatoid diseases, rheumatoid arthritis (Tisch R, McDevitt H O. Proc Natl Acad Sci USA Jan. 18, 1994; 91 (2): 437), systemic diseases, systemic autoimmune diseases, systemic lupus erythematosus (Datta SK., Lupus 1998; 7 (9): 591), glandular diseases, glandular autoimmune diseases, pancreatic diseases, pancreatic autoimmune diseases, Type 1 diabetes (Castano L. and Eisenbarth G S. Ann Rev. Immunol. 8: 647); thyroid diseases, autoimmune thyroid diseases, Graves' disease (Sakata S. et al., Mol Cell Endocrinol March 1993; 92 (1): 77); ovarian diseases (Garza K M. et al., J Reprod Immunol February 1998; 37 (2): 87), prostatitis, autoimmune prostatitis (Alexander R B. et al., Urology December 1997; 50 (6): 893), polyglandular syndrome, autoimmune polyglandular syndrome, Type I autoimmune polyglandular syndrome (Hara T. et al., Blood. Mar. 1, 1991; 77 (5): 1127), neurological diseases, autoimmune neurological diseases, multiple sclerosis, neuritis, optic neuritis (Soderstrom M. et al., J Neurol Neurosurg Psychiatry May 1994; 57 (5): 544), myasthenia gravis (Oshima M. et al., Eur J Immunol December 1990; 20 (12): 2563), stiffman syndrome (Hiemstra H S. et al., Proc Natl Acad Sci USA Mar. 27, 2001; 98 (7): 3988), cardiovascular diseases, cardiac autoimmunity in Chagas' disease (Cunha-Neto E. et al., J Clin Invest Oct. 15, 1996; 98 (8): 1709), autoimmune thrombocytopenic purpura (Semple J W. et al., Blood May 15, 1996; 87 (10): 4245), anti-helper T lymphocyte autoimmunity (Caporossi A P. et al., Viral Immunol 1998; 11 (1): 9), hemolytic anemia (Sallah S. et al., Ann Hematol March 1997; 74 (3): 139), hepatic diseases, hepatic autoimmune diseases, hepatitis, chronic active hepatitis (Franco A. et al., Clin Immunol Immunopathol March 1990; 54 (3): 382), biliary cirrhosis, primary biliary cirrhosis (Jones D E. Clin Sci (Colch) November 1996; 91 (5): 551), nephric diseases, nephric autoimmune diseases, nephritis, interstitial nephritis (Kelly C J. J Am Soc Nephrol August 1990; 1 (2): 140), connective tissue diseases, ear diseases, autoimmune connective tissue diseases, autoimmune ear disease (Yoo T J. et al., Cell Immunol August 1994; 157 (1): 249), disease of the inner ear (Gloddek B. et al., Ann N Y Acad Sci Dec. 29, 1997; 830: 266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption. Examples of types of T lymphocyte mediating hypersensitivity include, but are not limited to, helper T lymphocytes and cytotoxic T lymphocytes. Examples of helper T lymphocyte-mediated hypersensitivity include, but are not limited to, T.sub.h1 lymphocyte mediated hypersensitivity and T.sub.h2 lymphocyte mediated hypersensitivity.

According to some aspects, the disclosed peptides can be used to treat an inflammation associated with an autoimmune disease. Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases. According to some aspects, the disclosed peptides can be used to treat autoimmune gastrointestinal diseases, such as colitis.

Examples of autoimmune cardiovascular diseases include, but are not limited to atherosclerosis (Matsuura E. et al., Lupus. 1998; 7 Suppl 2: S135), myocardial infarction (Vaarala O. Lupus. 1998; 7 Suppl 2: S132), thrombosis (Tincani A. et al., Lupus 1998; 7 Suppl 2: S107-9), Wegener's granulomatosis, Takayasu's arteritis, Kawasaki syndrome (Praprotnik S. et al., Wien Klin Wochenschr Aug. 25, 2000; 112 (15-16): 660), anti-factor VIII autoimmune disease (Lacroix-Desmazes S. et al., Semin Thromb Hemost. 2000; 26 (2): 157), necrotizing small vessel vasculitis, microscopic polyangiitis, Churg and Strauss syndrome, pauci-immune focal necrotizing and crescentic glomerulonephritis (Noel L H. Ann Med Interne (Paris). May 2000; 151 (3): 178), antiphospholipid syndrome (Flamholz R. et al., J Clin Apheresis 1999; 14 (4): 171), antibody-induced heart failure (Wallukat G. et al., Am J. Cardiol. Jun. 17, 1999; 83 (12A): 75H), thrombocytopenic purpura (Moccia F. Ann Ital Med. Int. April-June 1999; 14 (2): 114; Semple J W. et al., Blood May 15, 1996; 87 (10): 4245), autoimmune hemolytic anemia (Efremov D G. et al., Leuk Lymphoma January 1998; 28 (3-4): 285; Sallah S. et al., Ann Hematol March 1997; 74 (3): 139), cardiac autoimmunity in Chagas' disease (Cunha-Neto E. et al., J Clin Invest Oct. 15, 1996; 98 (8): 1709) and anti-helper T lymphocyte autoimmunity (Caporossi A P. et al., Viral Immunol 1998; 11 (1): 9).

Examples of autoimmune rheumatoid diseases include, but are not limited to rheumatoid arthritis (Krenn V. et al., Histol Histopathol July 2000; 15 (3): 791; Tisch R, McDevitt H O. Proc Natl Acad Sci units S A Jan. 18, 1994; 91 (2): 437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann Rev. Immunol. 8: 647; Zimmet P. Diabetes Res Clin Pract October 1996; 34 Suppl: S125), autoimmune thyroid diseases, Graves' disease (Orgiazzi J. Endocrinol Metab Clin North Am June 2000; 29 (2): 339; Sakata S. et al., Mol Cell Endocrinol March 1993; 92 (1): 77), spontaneous autoimmune thyroiditis (Braley-Mullen H. and Yu S, J Immunol Dec. 15, 2000; 165 (12): 7262), Hashimoto's thyroiditis (Toyoda N. et al., Nippon Rinsho August 1999; 57 (8): 1810), idiopathic myxedema (Mitsuma T. Nippon Rinsho. August 1999; 57 (8): 1759), ovarian autoimmunity (Garza K M. et al., J Reprod Immunol February 1998; 37 (2): 87), autoimmune anti-sperm infertility (Diekman A B. et al., Am J Reprod Immunol. March 2000; 43 (3): 134), autoimmune prostatitis (Alexander R B. et al., Urology December 1997; 50 (6): 893) and Type I autoimmune polyglandular syndrome (Hara T. et al., Blood. Mar. 1, 1991; 77 (5): 1127).

Examples of autoimmune gastrointestinal diseases include, but are not limited to, chronic inflammatory intestinal diseases (Garcia Herola A. et al., Gastroenterol Hepatol. January 2000; 23 (1): 16), celiac disease (Landau Y E. and Shoenfeld Y. Harefuah Jan. 16, 2000; 138 (2): 122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol March 1990; 54 (3): 382), primary biliary cirrhosis (Jones D E. Clin Sci (Colch) November 1996; 91 (5): 551; Strassburg C P. et al., Eur J Gastroenterol Hepatol. June 1999; 11 (6): 595) and autoimmune hepatitis (Manns M P. J Hepatol August 2000; 33 (2): 326).

Examples of autoimmune neurological diseases include, but are not limited to, multiple sclerosis (Cross A H. et al., J Neuroimmunol Jan. 1, 2001; 112 (1-2): 1), Alzheimer's disease (Oron L. et al., J Neural Transm Suppl. 1997; 49: 77), myasthenia gravis (Infante A J. And Kraig E, Int Rev Immunol 1999; 18 (1-2): 83; Oshima M. et al., Eur J Immunol December 1990; 20 (12): 2563), neuropathies, motor neuropathies (Komberg A J. J Clin Neurosci. May 2000; 7 (3): 191); Guillain-Barre syndrome and autoimmune neuropathies (Kusunoki S. Am J Med. Sci. April 2000; 319 (4): 234), myasthenia, Lambert-Eaton myasthenic syndrome (Takamori M. Am J Med. Sci. April 2000; 319 (4): 204); paraneoplastic neurological diseases, cerebellar atrophy, paraneoplastic cerebellar atrophy and stiff-man syndrome (Hiemstra H S. et al., Proc Natl Acad Sci units SA Mar. 27, 2001; 98 (7): 3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) January 2000; 156 (1): 23); dysimmune neuropathies (Nobile-Orazio E. et al., Electroencephalogr Clin Neurophysiol Suppl 1999; 50: 419); acquired neuromyotonia, arthrogryposis multiplex congenita (Vincent A. et al., Ann N Y Acad. Sci. May 13, 1998; 841: 482), neuritis, optic neuritis (Soderstrom M. et al., J Neurol Neurosurg Psychiatry May 1994; 57 (5): 544) and neurodegenerative diseases.

Examples of autoimmune muscular diseases include, but are not limited to, myositis, autoimmune myositis and primary Sjogren's syndrome (Feist E. et al., Int Arch Allergy Immunol September 2000; 123 (1): 92) and smooth muscle autoimmune disease (Zauli D. et al., Biomed Pharmacother June 1999; 53 (5-6): 234).

Examples of autoimmune nephric diseases include, but are not limited to, nephritis and autoimmune interstitial nephritis (Kelly C J. J Am Soc Nephrol August 1990; 1 (2): 140).

Examples of autoimmune diseases related to reproduction include, but are not limited to, repeated fetal loss (Tincani A. et al., Lupus 1998; 7 Suppl 2: S107-9).

Examples of autoimmune connective tissue diseases include, but are not limited to, ear diseases, autoimmune ear diseases (Yoo T J. et al., Cell Immunol August 1994; 157 (1): 249) and autoimmune diseases of the inner ear (Gloddek B. et al., Ann N Y Acad Sci Dec. 29, 1997; 830: 266).

Examples of autoimmune systemic diseases include, but are not limited to, systemic lupus erythematosus (Erikson J. et al., Immunol Res 1998; 17 (1-2): 49) and systemic sclerosis (Renaudineau Y. et al., Clin Diagn Lab Immunol. March 1999; 6 (2): 156); Chan 0 T. et al., Immunol Rev June 1999; 169: 107).

According to some aspects of the disclosed method, the disclosed peptides can be used to treat an inflammation associated with infectious diseases. Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

According to some aspects of the disclosed method, the disclosed peptides can be used to treat an inflammation associated with a disease associated with transplantation of a graft. Examples of diseases associated with transplantation of a graft include, but are not limited to, graft rejection, chronic graft rejection, subacute graft rejection, hyperacute graft rejection, acute graft rejection and graft versus host disease. Types of grafts whose rejection can be treated by the disclosed method include, but are not limited to, syngeneic grafts, allografts and xenografts. According to some aspects, the disclosed peptides can be used to treat allograft rejection. Examples of grafts include cellular grafts, tissue grafts, organ grafts and appendage grafts. Examples of cellular grafts include, but are not limited to, stem cell grafts, progenitor cell grafts, hematopoietic cell grafts, embryonic cell grafts and a nerve cell grafts. Examples of tissue grafts include, but are not limited to, skin grafts, bone grafts, nerve grafts, intestine grafts, corneal grafts, cartilage grafts, cardiac tissue grafts, cardiac valve grafts, dental grafts, hair follicle grafts and muscle grafts. Examples of organ grafts include, but are not limited to, kidney grafts, heart grafts, skin grafts, liver grafts, pancreatic grafts, lung grafts and intestine grafts. Examples of appendage grafts include, but are not limited to, arm grafts, leg grafts, hand grafts, foot grafts, finger grafts, toe grafts and sexual organ grafts. According to some aspects, the disclosed peptides can be used to treat kidney allograft rejection.

According to some aspects of the disclosed method, the disclosed peptides can be used to treat inflammation associated with allergic diseases. Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy. For example, the disclosed peptides can be used, according to the disclosed method, to treat asthma.

According to some aspects of the disclosed method, the disclosed peptides can be used to treat inflammations associated with neurodegenerative diseases.

According to some aspects of the disclosed method, the disclosed peptides can be used to treat inflammations associated with cardiovascular diseases.

According to some aspects of the disclosed method, the disclosed peptides can be used to treat inflammations associated with gastrointestinal diseases. Examples of gastrointestinal diseases include, but are not limited to, the examples of antibody-mediated gastrointestinal diseases listed hereinabove, the examples of T lymphocyte-mediated gastrointestinal diseases listed hereinabove, the examples of autoimmune gastrointestinal diseases listed hereinabove and hemorrhoids. According to some aspects of the disclosed method, the disclosed peptides can be used to treat colitis.

According to some aspects of the disclosed method, the disclosed peptides can be used to treat inflammations associated with neurodegenerative diseases.

According to some aspects of the disclosed method, the disclosed peptides can be used to treat inflammations associated with tumors. Examples of tumors include, but are not limited to, malignant tumors, benign tumors, solid tumors, metastatic tumors and non-solid tumors. According to some aspects of the disclosed method, the disclosed peptides can be used to treat inflammation associated with septic shock.

According to some aspects of the disclosed method, the disclosed peptides can be used to treat inflammation associated with anaphylactic shock. According to some aspects of the disclosed method, the disclosed peptides can be used to treat inflammation associated with toxic shock syndrome.

According to some aspects of the disclosed method, the disclosed peptides can be used to treat inflammation associated with cachexia. According to some aspects of the disclosed method, the disclosed peptides can be used to treat inflammation associated with necrosis. According to some aspects of the disclosed method, the disclosed peptides can be used to treat inflammation associated with gangrene.

According to some aspects of the disclosed method, the disclosed peptides can be used to treat inflammations associated with prosthetic implants. Examples of prosthetic implants include, but are not limited to, breast implants, silicone implants, dental implants, penile implants, cardiac implants, artificial joints, bone fracture repair devices, bone replacement implants, drug delivery implants, catheters, pacemakers, respirator tubes and stents.

According to some aspects of the disclosed method, the disclosed peptides can be used to treat inflammation associated with menstruation.

According to some aspects of the disclosed method, the disclosed peptides can be used to treat inflammations associated with ulcers.

Examples of ulcers include, but are not limited to, skin ulcers, bed sores, gastric ulcers, peptic ulcers, buccal ulcers, nasopharyngeal ulcers, esophageal ulcers, duodenal ulcers, ulcerative colitis and gastrointestinal ulcers.

According to some aspects of the disclosed method, the disclosed peptides can be used to treat inflammations associated with injuries. Examples of injuries include, but are not limited to, abrasions, bruises, cuts, puncture wounds, lacerations, impact wounds, concussions, contusions, thermal burns, frostbite, chemical burns, sunburns, dessications, radiation bums, radioactivity burns, smoke inhalation, torn muscles, pulled muscles, torn tendons, pulled tendons, pulled ligaments, torn ligaments, hyperextensions, torn cartilage, bone fractures, pinched nerves and a gunshot wounds.

According to some aspects of the disclosed method, the disclosed peptides can be used to treat musculo-skeletal inflammations. Examples of musculo-skeletal inflammations include, but are not limited to, muscle inflammations, myositis, tendon inflammations, tendinitis, ligament inflammations, cartilage inflammation, joint inflammations, synovial inflammations, carpal tunnel syndrome and bone inflammations.

According to some aspects of the disclosed method, the disclosed peptides can be used to treat idiopathic inflammations.

According to some aspects of the disclosed method, the disclosed peptides can be used to treat inflammations of unknown etiology.

ii. Neurodegenerative Disease

The condition or disease can in some aspects be a neurodegenerative disease. Thus, provided is a method of treating, preventing, or reducing the risk of developing a neurodegenerative disorder, such as Alzheimer's disease, in a subject comprising administering to the subject a therapeutically effective amount of a composition comprising the disclosed peptide or a nucleic acid encoding the disclosed peptide. Also provided is a method of treating a subject at risk for a neurodegenerative disorder, such as Alzheimer's disease, comprising administering to the subject a composition comprising the disclosed peptide or a nucleic acid encoding the disclosed peptide. As used herein, the terms “disorder” and “disease” are used interchangeably to refer to a condition in a subject.

As used herein, the term “Aβ-related disorder” or an “Aβ disorder” is a disease (e.g., Alzheimer's disease) or a condition (e.g., senile dementia) that involves an aberration or dysregulation of Aβ levels. An Aβ-related disorder includes, but is not limited to Alzheimer's disease, Down's syndrome and inclusion body myositis. Thus, the Aβ related disorder can be Alzheimer's disease. The progression of the Aβ related disorder can be slowed or reversed.

Also provided is a method for modulating amyloid-β peptide (Aβ) levels exhibited by a cell or tissue comprising contacting said cell or tissue with an amount of a composition comprising the disclosed peptide or a nucleic acid encoding the disclosed peptide, sufficient to modulate said Aβ levels.

As used herein, a cell or tissue may include, but not be limited to: an excitable cell, e.g., a sensory neuron, motomeuron, or interneuron; a glial cell; a primary culture of cells, e.g., a primary culture of neuronal or glial cells; cell(s) derived from a neuronal or glial cell line; dissociated cell(s); whole cell(s) or intact cell(s); permeabilized cell(s); a broken cell preparation; an isolated and/or purified cell preparation; a cellular extract or purified enzyme preparation; a tissue or organ, e.g., brain, brain structure, brain slice, spinal cord, spinal cord slice, central nervous system, peripheral nervous system, or nerve; tissue slices, and a whole animal. In certain embodiments, the brain structure is cerebral cortex, the hippocampus, or their anatomical and/or functional counterparts in other mammalian species. In certain embodiments, the cell or tissue is an N2a cell, a primary neuronal culture or a hippocampal tissue explant.

Also provided is a method for prevention, treatment, e.g., management, of an Aβ-related disorder, or amelioration of a symptom of an Aβ-related disorder such as Alzheimer's disease. It is understood that the methods described herein in the context of treating and/or ameliorating a symptom can also routinely be utilized as part of a prevention protocol.

Also provided is a method of treating, or ameliorating a symptom of, an Aβ-related disorder comprising administering to a subject in need of such treating or ameliorating an amount of a composition comprising the disclosed peptide or a nucleic acid encoding the disclosed peptide sufficient to reduce Aβ levels in the subject such that the Aβ-related disorder is treated or a symptom of the Aβ related disorder is ameliorated.

Examples of neurodegenerative disorders include Alexander disease, Alper's disease, Alzheimer disease, Amyotrophic lateral sclerosis, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington disease, Kennedy's disease, Krabbe disease, Lewy body dementia, Machado-Joseph disease, Spinocerebellar ataxia type 3, Multiple sclerosis, Multiple System Atrophy, Parkinson's disease, Pelizaeus-Merzbacher Disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Transmissible spongiform encephalopathies (TSE), and Tabes dorsalis.

The condition or disease can in some aspects be Alzheimer's disease. Alzheimer's disease is a progressive neurodegenerative disorder that is characterized by the formation of senile plaques and neurofibrillary tangles containing amyloid β (Aβ) peptide. These plaques are found in limbic and association cortices of the brain. The hippocampus is part of the limbic system and plays an important role in learning and memory. In subjects with Alzheimer's disease, accumulating plaques damage the neuronal architecture in limbic areas and eventually cripple the memory process.

iii. Metabolic Disease

The condition or disease can in some aspects be a metabolic disease. Metabolic disease refers to diabetes and disorders of carbohydrate metabolism, amino acid metabolism, organic acid metabolism (organic acidurias), fatty acid oxidation and mitochondrial metabolism, porphyrin metabolism, purine or pyrimidine metabolism, steroid metabolism, mitochondrial function, peroxisomal function, Lysosomal storage disorders, Acromegaly, Addison's Disease, Cushing's Syndrome, Cystic Fibrosis, Endocrine Diseases, Human Growth Hormone related diseases, Hyperparathyroidism, Multiple Endocrine Neoplasia Type 1, Prolactinoma, Turner Syndrome.

Thus, the condition or disease can in some aspects be diabetes. The World Health Organization recognizes three main forms of diabetes: type 1, type 2, and gestational diabetes (occurring during pregnancy), which have similar signs, symptoms, and consequences, but different causes and population distributions. Type 1 is usually due to autoimmune destruction of the pancreatic beta cells which produce insulin. Type 2 is characterized by tissue-wide insulin resistance and varies widely; it sometimes progresses to loss of beta cell function. Gestational diabetes is similar to type 2 diabetes, in that it involves insulin resistance. The hormones of pregnancy cause insulin resistance in those women genetically predisposed to developing this condition. Types 1 and 2 are incurable chronic conditions, but have been treatable since insulin became medically available in 1921. Gestational diabetes typically resolves with delivery. Thus, in some aspects of the disclosed method, the subject has been diagnosed with or is at risk for type 1 diabetes mellitus.

Thus, provided is a method of treating or preventing diabetes in a subject, comprising administering to the subject a composition comprising the disclosed peptide or a nucleic acid encoding the disclosed peptide.

### 3. Screening Assays

Also provided herein is a method of identifying an inhibitor of inflammation. The method can comprise preparing a sample comprising a Nod-Like Receptor (NLR), the disclosed peptide, and a candidate agent, and detecting the binding of the peptide to the NLR. A decrease in the binding of the peptide and the NLR as compared to a control is an indication that the candidate agent is an inhibitor of inflammation. For example, the method can comprise preparing a sample comprising a Nod-Like Receptor (NLR), any of the disclosed peptides, and a candidate agent, detecting the binding of the isolated peptide to the NLR, wherein a decrease in the binding of the isolated peptide and the NLR as compared to a control is an indication that the candidate agent is an inhibitor of inflammation. As another example, the method can comprise preparing a sample comprising a Nod-Like Receptor (NLR), any of the disclosed peptides that can bind to a NLR, and a candidate agent, detecting the binding of the isolated peptide to the NLR, wherein a decrease in the binding of the isolated peptide and the NLR as compared to a control is an indication that the candidate agent is an inhibitor of inflammation.

As another example, the method can comprise preparing a sample comprising a Nod-Like Receptor (NLR), an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and a candidate agent, detecting the binding of the isolated peptide to the NLR, wherein a decrease in the binding of the isolated peptide and the NLR as compared to a control is an indication that the candidate agent is an inhibitor of inflammation. As another example, the method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 8 amino acids in length that can bind to the NLR, and a candidate agent, and detecting the binding of the peptide to the NLR. A decrease in the binding of the peptide and the NLR as compared to a control is an indication that the candidate agent is an inhibitor of inflammation.

Because of the relationship between inflammation, IL-1β production, IL-18 production, IL-33 production, caspase-1 activity, inflammasome activity, inflammasome assembly, NLR activity, and NLR ATP binding, a similar method can be used to identify an inhibitor of IL-1β production, IL-18 production, IL-33 production, caspase-1 activity, inflammasome activity, inflammasome assembly, NLR activity, and NLR ATP binding. In such methods, a decrease in the binding of the isolated peptide and the NLR as compared to a control is an indication that the candidate agent is an inhibitor of IL-1β production, IL-18 production, IL-33 production, caspase-1 activity, inflammasome activity, inflammasome assembly, NLR activity, and NLR ATP binding.

The binding of the peptide to the NLR can be measured by fluorescent polarization assay (FPA), time resolved fluorescent resonance energy transfer (TR-FRET), and/or scintillation proximity assay (SPA).

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the pathways and diseases disclosed herein.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity disclosed herein. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of the disclosed peptides.

Also provided is a method of identifying a modulator of Nod-like Receptor activity. The method can comprise preparing a sample comprising a Nod-Like Receptor (NLR), the disclosed peptide, and a candidate agent, and detecting the binding of the peptide to the NLR. A decrease in the binding of the peptide and the NLR as compared to a control is an indication that the candidate agent is an inhibitor of NLR activity. The method can comprise preparing a sample comprising a Nod-Like Receptor (NLR), a peptide comprising an amino acid sequence having at least 70% identity to the sequence set forth in SEQ ID NO:2, SEQ ID NO:3 or SEQ ID NO:4 that can bind to the NLR, or a fragment of the sequence set forth in SEQ ID NO:2, SEQ ID NO:3 or SEQ ID NO:4 of at least 8 amino acids in length that can bind to the NLR, and a candidate agent, and detecting the binding of the peptide to the NLR. A decrease in the binding of the peptide and the NLR as compared to a control is an indication that the candidate agent is an inhibitor of NLR activity.

Also provided is a process for making a modulator of Nod-like Receptor (NLR), the method comprising manufacturing the compound identified by the method disclosed herein.

Also disclosed are methods of identifying inhibitory sites on Nod-Like Receptors (NLRs). For example, the method can comprise bringing into contact a NLR and any of the disclosed peptides, and detecting the location where the peptide binds the NLR. As another example, the method can comprise bringing into contact a NLR and any of the disclosed peptides that can bind to a NLR, and detecting the location where the peptide binds the NLR. As another example, the method can comprise bringing into contact a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and detecting the location where the peptide binds the NLR. As another example, the method can comprise bringing into contact a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, and detecting the location where the peptide binds the NLR. The location where the peptide binds the NLR can be detected using, for example, nuclear magnetic resonance. The location where the peptide binds the NLR can be detected using, for example x-ray crystallography.

Also disclosed are methods of identifying compounds that can bind to a Nod-Like Receptor (NLR). For example, the method can comprise modeling the interaction of a NLR and any of the disclosed peptides, selecting or designing compounds predicted to have similar contacts with the NLR as the peptide, and assessing the interaction of the compound using modeling software and/or a modeling system. As another example, the method can comprise modeling the interaction of a NLR and any of the disclosed peptides that can bind to a Nod-Like Receptor (NLR), selecting or designing compounds predicted to have similar contacts with the NLR as the peptide, and assessing the interaction of the compound using modeling software and/or a modeling system. As another example, the method can comprise modeling the interaction of a NLR and any of the disclosed peptides that can compete with F1L, Bcl-2, or both for binding a Nod-Like Receptor (NLR), selecting or designing compounds predicted to have similar contacts with the NLR as the peptide, and assessing the interaction of the compound using modeling software and/or a modeling system. As another example, the method can comprise modeling the interaction of a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, selecting or designing compounds predicted to have similar contacts with the NLR as the peptide, and assessing the interaction of the compound using modeling software and/or a modeling system. As another example, the method can comprise modeling the interaction of a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, selecting or designing compounds predicted to have similar contacts with the NLR as the peptide, and assessing the interaction of the compound using modeling software and/or a modeling system.

### C. DETECTION

Binding and dissociation of the disclosed peptides and other components described herein can be detected using routine methods. In some aspects, one or more components, such as the disclosed peptide, of the present methods comprise a tag. By “tag” is meant an attached molecule or molecules useful for the identification or isolation of the attached component. Components having a tag are referred to as “tag-X”, wherein X is the component. For example, a peptide comprising a tag is referred to herein as “tag-peptide.” Moreover, reference to a component is also a reference to that component attached to a tag. For example, reference to peptide is also a reference to tag-peptide, such as His-peptide, which can be used, for example, to isolate, purify, or identify the peptide.

For example, disclosed are methods of detecting Nod-Like Receptors (NLRs). For example, the method can comprise (a) bringing into contact a sample and any of the disclosed peptides, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. As another example, the method can comprise (a) bringing into contact a sample and any of the disclosed peptides that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate.

As another example, the method can comprise (a) bringing into contact a sample and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. As another example, the method can comprise (a) bringing into contact a sample and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 8 amino acids in length that can bind to a NLR, wherein the peptide is conjugated to a first tag, wherein the first tag is in a binding pair with a second tag, (b) prior to, simultaneous with, or following step (a), bringing into contact the peptide and a surface substrate comprising the second tag, wherein the conjugated peptide binds to the second tag, and (c) detecting NLR associated with the surface substrate. The first tag can be, for example, biotin and the second tag can be streptavidin or avidin, or vice versa. The support surface can comprise a bead, a plate, or a multi-well plate. The NLR can be detected via, for example, an enzyme-linked immunosorbent assay.

The tag can be covalently bound to the attached component. When more than one component of a combination has a tag, the tags can be numbered for identification, for example “tag1-peptide”. Components can comprise more than one tag, in which case each tag can be numbered, for example “tag1,2-peptide”. Exemplary tags include, but are not limited to, a label, a partner of a binding pair, and a surface substrate binding molecule. As will be evident to the skilled artisan, many molecules can find use as more than one type of tag, depending upon how the tag is used.

Thus, binding and dissociation of the disclosed peptides and other components can be detected by detecting tagged peptide or component either in the bound state or in the dissociated state. In some aspects, tag1 and tag2 are fluorescent labels constituting a fluorescence resonance energy transfer (FRET) pair. In some aspects, detection is performed in a multi-well plate comprising a surface substrate comprising nickel.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is done can depend on the label. Exemplary labels include, but are not limited to, fluorescent labels, label enzymes and radioisotopes.

By “fluorescent label” is meant any molecule that an be detected via its inherent fluorescent properties. Suitable fluorescent labels include, but are not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO— TAGT™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydrorhodamine 123 (DHR); Dil (DilC18(3)); 1Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

By “label enzyme” is meant an enzyme which can be reacted in the presence of a label enzyme substrate which produces a detectable product. Suitable label enzymes for use in the present methods include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products can be opaque, such as the reaction of horseradish peroxidase with tetramethyl benzedine, and can have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

Fluorescence polarization is a detection method that ratios the intensities of vertically versus horizontally polarized fluorescence from a sample that has been illuminated with plane polarized light. Fluorescence polarization techniques have been described for the study of enzyme activity. A. Ping and J. Herron describe a competitive fluorescent polarization immunoassay wherein a fluorescent peptide substrate is displaced from an antibody by a natural substrate of interest (Anal. Chem., 65, 3372-3377 (1993)). U.S. Pat. No. 5,070,025, to Klein et al, describes a fluorescence polarization immunoassay. U.S. Pat. No. 4,640,893, to Mangel et al, describes rhodamine-peptide derivatives as fluorogenic protease substrates. H. Maeda describes the use of fluorescence polarization in the study of proteolytic enzyme cleavage of protein substrates (Anal. Biochem., 92, 222-227 (1979)). H. Maeda describes the use of fluorescence polarization in the study of lysozyme cleavage of an isolated peptidoglycan natural product (J. Biochem., 88, 1185-1191 (1980)).

Scintillation proximity assay (SPA) makes use of the limited pathlength of certain electron-emitters (Hart et al., Molecular Immunology 1979 16:265-267; Hart, U.S. Pat. Nos. 4,271,139 and 4,382,074; and Bertoglio-Matte, U.S. Pat. No. 4,568,649). An exemplary SPA is composed of an analyte in solution, plastic beads which scintillate when exposed to electrons, and a specific binding partner (such as an antibody) bound to the beads and specific for the analyte in solution. If the analyte incorporates a radioactive label which emits electrons of relatively short pathlength, such as tritium, the plastic beads will only scintillate when suspended in solution with the radioactive analyte when the analyte is specifically bound by the binding partner and thus localized near the surface of the beads.

SPAs have been developed and exploited for a variety of analytical purposes. SPAs have been used for radioimmunoassays, competition assays, enzyme kinetic assays, studies of ligand/receptor and antigen/antibody interactions, and studies of cellular processes (see, Cook, Drug Discovery Today 1996 1:287-294; and Cook, U.S. Pat. No. 5,665,562). The SPAs described to date all rely on specific binding interactions, such as antibody-antigen interactions, ligand-receptor interactions, biotinylated reagents which bind to streptavidin-coated beads, chelate complex formation of the species of interest, or other interactions which rely on the precise and specific structural complementarity of binding partners.

Scintillation Proximity Assay (SPA) can be an homogeneous assay procedure which produces quantifiable light energy at a level which is related to the amount of radioactively labelled product in the assay medium. The light energy is produced by a scintillant which is either incorporated, or forms part of, a support structure (beads or other solid surface which can be used in the assay process). While the support structure may be coated with a capture molecule, capture molecules are not necessary for the practice of the present invention. In a direct assay, a sample containing a radiolabelled product is mixed in aqueous solution containing scintillant support structure. The radiolabelled product is caused to bind to the scintillant-containing support structure. The scintillant is activated causing emission of light, which can be detected conventionally using a scintillation counter. The amount of light produced is directly proportional to the amount of reactant bound to the surface of the support structures. Beads that are used in SPA can be microspheres, approximately 5 um in diameter, and can be made from hydrophobic polymers such as but not limited to polyacrylamide, acrylamide, agarose, polystyrene, polypropylene, polycarbonate, and polyvinyltoluene or from inorganic scintillators such as yttrium silicate. The core of the bead can be coated with a polyhydroxy film which reduces the hydrophobicity of the bead. In some aspects, SPA beads are made from either yttrium silicate or polyvinyltoluene containing an organic scintillant such as diphenyloxazole and are commercially available from Amersham Biosciences (Piscataway, N.J.).

1. FRET

In some instances, multiple fluorescent labels are used. In some aspects, at least two fluorescent labels are used which are members of a Fluorescence (Förster) Resonance Energy Transfer (FRET) pair. FRET refers to an energy transfer mechanism between two chromophores. A donor chromophore in its excited state can transfer energy by a nonradiative, long-range dipole-dipole coupling mechanism to an acceptor chromophore in close proximity (typically <10 nm).

A FRET pair consists of a donor fluorophore and an acceptor fluorophore.

The fluorescence emission spectrum of the donor and the fluorescence absorption spectrum of the acceptor must overlap, and the two molecules must be in close proximity The distance between donor and acceptor at which 50% of donors are deactivated (transfer energy to the acceptor) is defined by the Förster radius (Rₒ), which is typically 10-100A. Changes in the fluorescence emission spectrum comprising FRET pairs can be detected, indicating changes in the number of that are in close proximity (i.e., within 100 Å of each other). This will typically result from the binding or dissociation of two molecules, one of which is labeled with a FRET donor and the other of which is labeled with a FRET acceptor, wherein such binding brings the FRET pair in close proximity. Binding of such molecules can result in an increased fluorescence emission of the acceptor and/or quenching of the fluorescence emission of the donor.

An example of a FRET pair for biological use is a cyan fluorescent protein (CFP)-yellow fluorescent protein (YFP) pair. Both are color variants of green fluorescent protein (GFP). While labeling with organic fluorescent dyes requires troublesome processes of purification, chemical modification, and intracellular injection of a host protein, GFP variants can be easily attached to a host protein by genetic engineering.

Other FRET pairs (donor/acceptor) useful in the present methods include, but are not limited to, EDANS/fluorescien, IAEDANS/fluorescein, fluorescein/tetramethylrhodamine, fluorescein/LC Red 640, fluorescein/Cy 5, fluorescein/Cy 5.5 and fluorescein/LC Red 705.

In another aspect of FRET, a fluorescent donor molecule and a nonfluorescent acceptor molecule (“quencher”) can be employed. In this application, fluorescent emission of the donor can increase when quencher is displaced from close proximity to the donor and fluorescent emission can decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DAB CYL, coumarin/DABCYL and fluorescein/QSY 7 dye.

Many compounds and proteins present in biological fluids or serum are naturally fluorescent, and the use of conventional, prompt fluorophores leads to serious limitations in assay sensitivity. The use of long-lived fluorophores combined with time-resolved detection (a delay between excitation and emission detection) minimizes prompt fluorescence interferences. Time-resolved fluorometry (TRF) takes advantage of the unique properties of the rare earth elements called lanthanides. The commonly used lanthanides in TRF assays are samarium (Sm), europium (Eu), terbium (Tb), and dysprosium (Dy). Because of their specific photophysical and spectral properties, complexes of rare earth ions are of major interest for fluorescence applications in biology. Specifically, they have large Stoke's shifts and extremely long emission half-lives (from μsec to msec) when compared to more traditional fluorophores. Thus, in some aspects the FRET pairs of the disclosed method are terbium and fluorescein.

It is difficult to generate fluorescence of lanthanide ions by direct excitation, because of the ions' poor ability to absorb light. Lanthanides can therefore be complexed with organic moieties that harvest light and transfer it to the lanthanide through intramolecular, non-radiative processes. Rare earth chelates and cryptates are examples of light-harvesting devices. The collected energy is transferred to the rare earth ion, which then emits its characteristic long-lived fluorescence.

Commercial systems are available from Wallac, Oy, Turku, Finland and Packard Instrument Company, Meriden, USA, which use lanthanide chelates as the donor label and dyes from the phycobiliprotein class e.g. allophycocyanin as the acceptor label. The lanthanide chelates have a luminescence lifetime in a range up to several milliseconds i.e. the acceptor emission can be observed for a corresponding length of time. Hence the energy released by lanthanide chelates is usually measured in a time window between 400-600 microseconds. This also inevitably means that there are also relatively long dead times. The stability of the lanthanide chelates is reduced under certain test conditions; thus for example a re-chelation can occur when complexing agents such as EDTA (ethylene-di-amino-tetra-acetic acid) are added.

U.S. Pat. No. 5,998,146 is incorporated herein by reference for the teaching of lanthanide chelate complexes, such as europium and terbium complexes, combined with fluorophores or quenchers. Ruthenium complexes can also be used for time-resolved fluorescent measurement where lumazine is used as the energy donor and a ruthenium complex is used as the energy acceptor. The dye “reactive blue” can also used as the resonance energy acceptor for ruthenium complexes. Reactive blue suppresses the fluorescence emitted by the ruthenium complex and hence the quantification is based on the suppressed fluorescence signal which was originally emitted by the ruthenium complex. Ruthenium complex known as “Fair Oaks Red™” can be used as the energy donor, and fast green or light green yellowish can be used as acceptors for ruthenium complexes.

Also disclosed are detection methods which additionally utilize a time-delayed measurement of the signal from a FRET system. The principle of time-resolved FRET measurements is essentially based on selecting a measuring window such that interfering background fluorescence, e.g., due to interfering substances in the sample, is not co-detected, but rather only the fluorescence generated or suppressed by the energy transfer is measured. The resulting fluorescence of the TR-FRET system can be determined by means of appropriate measuring devices. Such time-resolved detection systems use for example pulsed laser diodes, light emitting diodes (LEDs) or pulsed dye lasers as the excitation light source. The measurement occurs after an appropriate time delay i.e. after the interfering background signals have decayed.

FRET systems based on metallic complexes as energy donors and dyes from the class of phycobiliproteins as energy acceptors are known in the art. Established commercial systems (e.g. from Wallac, OY or Cis Bio Packard) use a FRET pair consisting of a lanthanide chelate as the metallic complex and a phycobiliprotein. The advantageous properties of the lanthanide-chelate complexes in particular of europium or terbium complexes are known and can be used in combination with quenchers as well as in combination with fluorophores.

TR-FRET unites TRF (Time-Resolved Fluorescence) and FRET (Fluorescence Resonance Energy Transfer) principles. This combination brings together the low background benefits of TRF with the homogeneous assay format of FRET. This powerful combination provides significant benefits to drug discovery researchers including assay flexibility, reliability, increased assay sensitivity, higher throughput and fewer false positive/false negative results. HTRF® is a TR-FRET based technology that uses the principles of both TRF and FRET. The HTRF® donor fluorophore is either Europium cryptate (Eu3+ cryptate) or Lumi4™-Tb (Tb2+ cryptate). Both donors have the long-lived emissions of lanthanides coupled with the stability of cryptate encapsulation. XL665, a modified allophycocyanin, is the HTRF® primary acceptor fluorophore.

When these two fluorophores are brought together by a biomolecular interaction, a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is present in the assay, light at 620 nm is detected even when the biomolecular interaction does not bring XL665 within close proximity.

2. Binding Pairs

In addition, labels can be indirectly detected, such as wherein the tag is a partner of a binding pair. By “partner of a binding pair” is meant one of a first and a second moiety, wherein said first and said second moiety have a specific binding affinity for each other. Suitable binding pairs for use in the method include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avid (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto.

As will be appreciated by those in the art, a partner of one binding pair can also be a partner of another binding pair. For example, an antigen (first moiety) can bind to a first antibody (second moiety) which can, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair can comprise a label. It will further be appreciated that this allows for a tag to be indirectly labeled upon the binding of a binding partner comprising a label. Attaching a label to a tag which is a partner of a binding pair, as just described, is referred to herein as “indirect labeling”.

By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present methods include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

Methods for labeling of proteins with radioisotopes are known in the art. For example, such methods are found in Ohta et al., Molec. Cell 3:535-541 (1999), which is hereby incorporated by reference in its entirety. By “radioisotope” is meant any radioactive molecule. Suitable radioisotopes for use in the method include, but are not limited to ¹⁴C, ³H, ³²P, ³³P, ³⁵S, ¹²⁵I and ¹³¹I. The use of radioisotopes as labels is well known in the art.

The functionalization of labels with chemically reactive groups such as thiols, amines, carboxyls, etc. is generally known in the art. In some aspects, the tag is functionalized to facilitate covalent attachment.

3. Tag Attachment

The covalent attachment of the tag can be either direct or via a linker. In some aspects, the linker is a relatively short coupling moiety, that is used to attach the molecules. A coupling moiety can be synthesized directly onto a component of the method, peptide for example, and contains at least one functional group to facilitate attachment of the tag. Alternatively, the coupling moiety can have at least two functional groups, which are used to attach a functionalized component to a functionalized tag, for example. In some aspects, the linker is a polymer. In this aspect, covalent attachment is accomplished either directly, or through the use of coupling moieties from the component or tag to the polymer. In some aspects, the covalent attachment is direct, that is, no linker is used. In this aspect, the component can contain a functional group such as a carboxylic acid which is used for direct attachment to the functionalized tag. It should be understood that the component and tag can be attached in a variety of ways, including those listed above. What is important is that manner of attachment does not significantly alter the functionality of the component. For example, in tag-peptide, the tag should be attached in such a manner as to allow the peptide to be covalently bound to other peptide to form polypeptide chains. As will be appreciated by those in the art, the above description of covalent attachment of a label and peptide applies equally to the attachment of virtually any two molecules of the present disclosure.

In some aspects, the tag is functionalized to facilitate covalent attachment. Thus, a wide variety of tags are commercially available which contain functional groups, including, but not limited to, isothiocyanate groups, amino groups, haloacetyl groups, maleimides, succinimidyl esters, and sulfonyl halides, all of which can be used to covalently attach the tag to a second molecule, as is described herein. The choice of the functional group of the tag can depend on the site of attachment to either a linker, as outlined above or a component of the method. Thus, for example, for direct linkage to a carboxylic acid group of a peptide, amino modified or hydrazine modified tags can be used for coupling via carbodiimide chemistry, for example using 1-ethyl-3-(3-dimethylaminopropyl)-carbodiimi-de (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In some aspects, the carbodiimide is first attached to the tag, such as is commercially available for many of the tags described herein.

In some aspects, the disclosed peptide is in the form of tag-peptide. In some aspects, the disclosed peptide is in the form of tag-peptide, wherein, tag is a partner of a binding pair. In some aspects, the disclosed peptide is in the form of tag-peptide, wherein the tag is a fluorescent label. In some aspects, the disclosed peptide and NLR are in the form of tag 1-peptide and tag2-NLR, wherein tag 1 and tag2 are the members of a FRET pair. In some aspects, the disclosed peptide and NLR are in the form of tag1-peptide and tag2-NLR, wherein tag 1 is a fluorescent label and tag2 is a quencher of the fluorescent label. In some aspects, when tag1-peptide and tag2-NLR bind, tag1 and tag2 are within 100 Å, 90 Å, 80 Å, 70 Å, 60 Å, 50 Å, 40 Å, 30 Å or less.

4. Surface Substrates

Surface substrates or surfaces are solid-state substrates, compositions, surfaces and/or supports with which molecules, such as hemagglutinin, hemagglutinin compositions and/or antibodies, can be associated. Molecules can be associated with surface substrates directly or indirectly. For example, molecules can be bound to the surface of a surface substrate or associated with capture agents (e.g., compounds or molecules that bind an analyte) immobilized on surface substrates. An array is a surface substrate to which multiple molecules have been associated in an array, grid, or other organized pattern.

Solid-state substrates for use in surface substrates can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and surface substrates can be porous or non-porous. A chip is a polygonal (e.g., rectangular, square, triangular, circular) small piece of material. Useful forms for solid-state substrates are plates, dishes, thin films, beads, or chips. A useful form for a solid-state substrate is a microtiter dish. In some embodiments, a multiwell glass slide can be employed.

An array can include a plurality of molecules immobilized at identified or predefined locations on the surface substrate. Each predefined location on the surface substrate generally has one type of component (that is, all the components at that location are the same). Alternatively, multiple types of components can be immobilized in the same predefined location on a surface substrate. Each location will have multiple copies of the given components. The spatial separation of different components on the surface substrate allows separate detection and identification.

Although useful, it is not required that the surface substrate be a single unit or structure. A set of molecules can be distributed over any number of surface substrates. For example, at one extreme, each component can be immobilized in a separate reaction tube or container, or on separate beads or microparticles.

Each of the components immobilized on the surface substrate can be located in a different predefined region of the surface substrate. The different locations can be different reaction chambers. Each of the different predefined regions can be physically separated from each other of the different regions. The distance between the different predefined regions of the surface substrate can be either fixed or variable. For example, in an array, each of the components can be arranged at fixed distances from each other, while components associated with beads will not be in a fixed spatial relationship. In particular, the use of multiple surface substrate units (for example, multiple beads) will result in variable distances.

Components can be associated or immobilized on a surface substrate at any density. Components can be immobilized to the surface substrate at a density exceeding 400 different components per cubic centimeter. Arrays of components can have any number of components. For example, an array can have at least 1,000 different components immobilized on the surface substrate, at least 10,000 different components immobilized on the surface substrate, at least 100,000 different components immobilized on the surface substrate, or at least 1,000,000 different components immobilized on the surface substrate.

### D. ADMINISTRATION

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of a composition comprising the disclosed peptide or a nucleic acid encoding the disclosed peptide used alone might range from about 1 μg/kg to up to 100 mg/kg of body weight or more per day, depending on the factors mentioned above.

Following administration of a disclosed composition, the efficacy of the therapeutic peptide or nucleic acid can be assessed in various ways well known to the skilled practitioner. For instance, one of ordinary skill in the art will understand that a composition disclosed herein is efficacious in treating or inhibiting Alzheimer's disease in a subject by observing that the composition reduces amyloid beta or prevents a further increase in plaque formation. Other indicators of therapeutic efficacy disclosed herein can be measured by methods that are known in the art, for example, using polymerase chain reaction assays to detect the presence of nucleic acid or antibody assays to detect the presence of protein in a sample (e.g., but not limited to, blood) from a subject or patient, or by measuring the level of circulating levels in the patient. Efficacy of the administration of the disclosed composition may also be determined by routine diagnostic means. For example, efficacy of the disclosed compositions for treating diabetes can be determined by monitoring blood sugar.

The compositions disclosed herein may be administered prophylactically to patients or subjects who are at risk for inflammation, neurodegenerative disease, cardiovascular disease, or diabetes or who have been newly diagnosed with inflammation, neurodegenerative disease, cardiovascular disease, or diabetes.

The disclosed compositions and methods can also be used for example as tools to isolate and test new drug candidates for a variety of inflammation related diseases, neurodegenerative disease, cardiovascular disease, or diabetes related diseases.

### E. KITS

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for modulating Nod-like Receptor activity, the kit comprising the disclosed peptide or a nucleic acid encoding the disclosed peptide. The kits also can contain materials for detecting Nod-like Receptor activity.

### F. USES

The disclosed compositions can be used in a variety of ways as research tools. Other uses are disclosed, apparent from the disclosure, and/or will be understood by those in the art.

### G. METHODS OF MAKING THE COMPOSITIONS

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. For example, disclosed are nucleic acids in SEQ ID NO:1. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO:1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO:1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO:1 and a sequence controlling the expression of the nucleic acid.

Disclosed are nucleic acid molecules produced by the process comprising linking in an operative way a nucleic acid molecule comprising a sequence encoding, for example, a peptide set forth in SEQ ID NO:2 and a sequence controlling an expression of the nucleic acid molecule.

Disclosed are nucleic acid molecules produced by the process comprising linking in an operative way a nucleic acid molecule comprising a sequence encoding a peptide having 80% identity to, for example, a peptide set forth in SEQ ID NO:2 and a sequence controlling an expression of the nucleic acid molecule.

Disclosed are nucleic acids produced by the process comprising linking in an operative way a nucleic acid molecule comprising a sequence encoding a peptide having 80% identity to, for example, a peptide set forth in SEQ ID NO:2, wherein any change is a conservative change, and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

Immune regulatory proteins such as CIITA, NAIP, IPAF, NOD1, NOD2, NALP1, and cryopyrin (also known as NALP3) are members of a family characterized by the presence of a NACHT nucleotide-binding domain (NBD) and leucine-rich repeats (LRRs). Members of this gene family encode a protein structure similar to the NB-LRR subgroup of disease-resistance genes in plants and are involved in the sensing of pathogenic products and the regulation of cell signaling and death. A variety of different names have been used to describe the products encoded by the NBD and LRR containing gene family, its subfamilies, and individual genes, including CATERPILLER (CLR), NOD-LRR, NACHT-LRR, NOD-like receptor, CARD, NALP, NOD, PAN, and PYPAF. A standardized nomenclature, NLR, was proposed, which stands for the nucleotide-binding domain and leucine-rich repeat containing gene family.

The NLR family includes several subfamilies distinguishable by their N-terminal effector domains. There are four recognizable NLR N-terminal domains: acidic transactivation domain, pyrin domain, caspase recruitment domain (CARD), and baculoviral inhibitory repeat (BIR)-like domains (see Table 4 for the human NLR genes). These N-terminal domains have been used by several groups to subdivide the NLR gene family, and there are now multiple names for each subfamily: the largest pyrin-containing subfamily has been named PAN, NALP, and PYPAF; members of the CARD-containing subfamily have been named CARDs or NODs; the BIR-containing subfamily has been named NAIP or BIRC.

A new nomenclature system for human and mouse NLR genes was agreed upon (see table available online at the web site immunity.com/cgi/content/full/28/3/285/DC1/for human and mouse NLR genes). The family name “nucleotide-binding domain and leucine-rich repeat containing” should be used to highlight these two evolutionarily conserved domains and to reflect the similarity of the NLR family to the plant NB-LRR proteins. Furthermore, a subfamily-derived nomenclature system based on the N-terminal effector domains should be used. Consequently, four subfamily designations have been approved: NLRA, NLR family, acidic domain containing; NLRB, NLR family, BIR domain containing; NLRC, NLR family, CARD domain containing; NLRP, NLR family, pyrin domain containing; NLRX, NLR family with no strong homology to the N-terminal domain of any other NLR subfamily member (Table 4). Each member within a subfamily is given a number, e.g., NLRP1. Four members of the NLR family, CIITA, NAIP, NOD1, and NOD2, have not been renamed. These four genes are associated with a large volume of literature, and it was agreed that renaming these would cause confusion in the literature. However, each of these genes has been given a subfamily alias to enable electronic data-retrieval systems to link these four genes to the NLR gene family. Clearly related genes, such as NLRP10 and Naip3-6, that do not encode NBD and/or LRR are included for completeness and historic reasons. The following abbreviations are used: AD, acidic activation domain CARD, caspase activating and recruitment domain; LRR, leucine-rich repeat; NACHT, domain present in NAIP, CITTA, HET-E, and TP-1; BIR, baculovirus inhibitor of apoptosis repeat; PYD, pyrin domain; and NAD, NACHT-associated domain.

TABLE 4
Designations for the Human NLR Family Members

| NLR | | | | Domain | Protein |
|---|---|---|---|---|---|
| Family | Symbol | Name | Other Names and Aliases | Organization | Sequence |
| NLRA | CIITA | class II, major | NLRA; MHC2TA; C2TA | (CARD)-AD-NACHT- | NP_000237 |
| | | histocompatibility complex, | | NADLRR | |
| | | transactivator | | | |
| NLRB | NAIP | NLR family, apoptosis | NLRB1; BIRC1; CLR5.1 | BIR3x-NACHT-LRR | NP_004527 |
| | | inhibitory protein | | | |
| NLRC | NOD1 | nucleotide-binding | NLRC1; CARD4; CLR7.1 | CARD-NACHT-NAD- | NP_006083 |
| | | oligomerization domain | | LRR | |
| | | containing 1 | | | |
| NLRC | NOD2 | nucleotide-binding | NLRC2; CARD15; CD; BLAU; IBD1; | CARD2x-NACHT-NAD- | NP_071445 |
| | | oligomerization domain | PSORAS1; CLR16.3 | LRR | |
| | | containing 2 | | | |
| NLRC | NLRC3 | NLR family, CARD domain | NOD3; CLR16.2 | CARD-NACHT-NAD- | NP_849172 |
| | | containing 3 | | LRR | |
| NLRC | NLRC4 | NLR family, CARD domain | CARD12; CLAN; CLR2.1; IPAF | CARD-NACHT-NAD- | NP_067032 |
| | | containing 4 | | LRR | |
| NLRC | NLRC5 | NLR family, CARD domain | NOD27; CLR16.1 | CARD-NACHT-NAD- | NP_115582 |
| | | containing 5 | | LRR | |
| NLRP | NLRP1 | NLR family, pyrin domain | NALP1; DEFCAP; NAC; CARD7; | PYD-NACHT-NAD- | NP_127497 |
| | | containing 1 | CLR17.1 | LRRFIIND-CARD | |
| NLRP | NLRP2 | NLR family, pyrin domain | NALP2; PYPAF2; NBS1; PAN1; | PYD-NACHT-NAD-LRR | NP_060322 |
| | | containing 2 | CLR19.9 | | |
| NLRP | NLRP3 | NLR family, pyrin domain | CIAS1; PYPAF1; Cryopyrin; CLR1.1; | PYD-NACHT-NAD-LRR | NP_004886 |
| | | containing 3 | NALP3 | | |
| NLRP | NLRP4 | NLR family, pyrin domain | NALP4; PYPAF4; PAN2; RNH2; | PYD-NACHT-NAD-LRR | NP_604393 |
| | | containing 4 | CLR19.5 | | |
| NLRP | NLRP5 | NLR family, pyrin domain | NALP5; PYPAF8; MATER; PAN11; | PYD-NACHT-NAD-LRR | NP_703148 |
| | | containing 5 | CLR19.8 | | |
| NLRP | NLRP6 | NLR family, pyrin domain | NALP6; PYPAF5; PAN3; CLR11.4 | PYD-NACHT-NAD-LRR | NP_612202 |
| | | containing 6 | | | |
| NLRP | NLRP7 | NLR family, pyrin domain | NALP7; PYPAF3; NOD12; PAN7; | PYD-NACHT-NAD-LRR | NP_996611 |
| | | containing 7 | CLR19.4 | | |
| NLRP | NLRP8 | NLR family, pyrin domain | NALP8; PAN4; NOD16; CLR19.2 | PYD-NACHT-NAD-LRR | NP_789781 |
| | | containing 8 | | | |
| NLRP | NLRP9 | NLR family, pyrin domain | NALP9; NOD6; PAN12; CLR19.1 | PYD-NACHT-NAD-LRR | NP_789790 |
| | | containing 9 | | | |
| NLRP | NLRP10 | NLR family, pyrin domain | NALP10; PAN5; NOD8; PYNOD; | PYD-NACHT-NAD | NP_789791 |
| | | containing 10 | CLR11.1 | | |
| NLRP | NLRP11 | NLR family, pyrin domain | NALP11; PYPAF6; NOD17; PAN10; | PYD-NACHT-NAD-LRR | NP_659444 |
| | | containing 11 | CLR19.6 | | |
| NLRP | NLRP12 | NLR family, pyrin domain | NALP12; PYPAF7; Monarch1; RNO2; | PYD-NACHT-NAD-LRR | NP_653288 |
| | | containing 12 | PAN6; CLR19.3 | | |
| NLRP | NLRP13 | NLR family, pyrin domain | NALP13; NOD14; PAN13; CLR19.7 | PYD-NACHT-NAD-LRR | NP_789780 |
| | | containing 13 | | | |
| NLRP | NLRP14 | NLR family, pyrin domain | NALP14; NOD5; PAN8; CLR11.2 | PYD-NACHT-NAD-LRR | NP_789792 |
| | | containing 14 | | | |
| NLRX | NLRX1 | NLR family member X1 | NOD9; CLR11.3 | X-NACHT-NAD-LRR | NP_078894 |

### H. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Activities” of a protein include, for example, transcription, translation, intracellular translocation, secretion, phosphorylation by kinases, cleavage by proteases, homophilic and heterophilic binding to other proteins.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the reduction can be a 10, 20, 30, 40, 50, 60, 70, 80, 90, 100%, or any amount of increase in between as compared to native or control levels.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder. Such amelioration only requires a reduction or alteration, not necessarily elimination.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### I. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. EXAMPLE 1

### Loop Domain-Dependent Suppression of ATP Binding and Oligomerization in NLRP1 Inflammasome

Recently, it was reported that anti-apoptotic proteins Bcl-2 and Bcl-XL are capable of binding and inhibiting NLRP1, thus suppressing Caspase-1 activation and IL-1b production in cultured cells and in mice (Bruey, Cell 2007). Structure-function studies demonstrated that a approximately 50 amino-acid flexible loop between the first and second a-helices of Bcl-2 and Bcl-XL is critical for their association with NLRP1 in cells. However, the biochemical mechanism by which Bcl-2 and Bcl-XL accomplish their inhibition of NLRP1 was unknown. Using an in vitro reconstituted system, employing purified recombinant proteins, we investigated the mechanism of NLRP1 suppression by Bcl-2 and Bcl-XL.

NLRP1 (NLR family, pyrin domain-containing 1) is contributor to innate immunity involved in intracellular sensing of pathogens as well as danger signals related to cell injury. NLRP1 is one of the core components of Caspase-1-activating platforms termed “inflammasomes”, which are involved in proteolytic processing of Interleukin-1b (IL-1β) and in cell death. It was discovered that anti-apoptotic proteins Bcl-2 and Bcl-X_{L} bind to and inhibit NLRP1 in cells. Using an in vitro reconstituted system employing purified recombinant proteins, the mechanism by which Bcl-2 and Bcl-XL inhibit NLRP1 was studied. Bcl-2 and Bcl-XL inhibited Caspase-1 activation induced by NLRP1 in a concentration-dependent manner, with Ki approximately 10 nM. Bcl-2 and Bcl-XL were also determined to inhibit ATP binding to NLRP1, which is required for oligomerization of NLR1, and Bcl-XL was demonstrated to interfere with NLRP1 oligomerization. Deletion of the flexible loop regions of Bcl-2 and Bcl-XL, which are located between the first and second a-helices of these anti-apoptotic proteins and which were previously shown to be required for binding NLRP1, abrogated ability to inhibit Caspase-1 activation, ATP binding and oligomerization of NLRP1. Conversely, synthetic peptides corresponding to the loop region of Bcl-2 were sufficient to potently inhibit NLRP1. These findings thus demonstrate that the loop domain is necessary and sufficient to inhibit NLRP1, providing novel insights into the mechanism by which anti-apoptotic proteins Bcl-2 and Bcl-XL inhibit NLRP1.

i. Experimental Procedures

Protein production and purification. His-tagged recombinant proteins, (His)6-NRLP1, (His)6-NRLP1ALRR, and (His)6-pro-Caspase-1 were produced from Sf21 cells using recombinant baculoviruses as previously described (Faustin et al., (2007) Mol Cell 25, 713-24). Proteins in complex with Ni²⁺-sepharose (2 ml for 2 Liters of culture) were washed with 300 ml of lysis buffer, 1 M NaCl, and 20 mM Imidazole. Then, proteins were eluted using an imidazole gradient (0-250 mM) and the eluted proteins were submitted to gel-filtration using Superdex 200 columns in 20 mM HEPES-KOH pH 7.5, 1 mM EDTA, 1 mM EGTA, 1.5 mM MgCl₂, 150 mM NaCl, 10 mM KCl, 0.1% CHAPS, supplemented with 1 mM DTT for NLRP1 but not for pro-Caspase-1. Fractions containing monomers were detected by UV absorbance at 280 nm, concentrated, and quickly frozen in dry ice/ethanol. Fraction size ˜150 kDa (His6-NLRP1), ˜50 kDa (His6-pro-Caspase-1) were retained corresponding to monomers. For expression and purification of Bcl-2 family proteins, GST-fusion proteins containing Bcl-X_{L}, Bcl-2, and Bcl-X_{L}ΔLoop (A44-84) lacking their C-terminal transmembrane domains (˜last 20 amino acids) were expressed from pGEX4T-1 plasmid in XL-1 Blue cells (Stratagene, Inc.) (Zhai et al., (2006) Cell Death Differ 13, 1419-21). GST-fusion proteins were purified by affinity chromatography using glutathione-Sepharose as described.

Caspase activity measurements. Recombinant proteins NLRP1 and pro-Caspase-1 were diluted in 20 mM HEPES pH 7.5, 150 mM NaCl, 0.1% CHAPS, 1 mM DTT, maintaining total protein constant with bovine serum albumin (BSA). Proteins were mixed in Caspase-1 buffer (100 mM HEPES, [pH 7.5], 10% Sucrose, 0.1% CHAPS, 1 mM Dithiothreitol) in a final volume of 10 μl in 96-well plates. Various activators were added (ATP, Mg²⁺, MDP-LD), adjusting final volume with water. Samples were then incubated for 30 min at 37° C. and Caspase-1 activity was measured using 20 μM fluorometric substrate Ac-WEHD-AMC (Calbiochem). Release of AMC product was monitored continuously by spectrofluorometry in kinetic mode over 1 h at 25-27° C. Rates of catalysis were calculated using the initial slope and Caspase activity was expressed as the change in fluorescence over time derived from the linear phase of reactions. Data were analyzed using the PRISM Statistics software, employing an unpaired t test.

Kinetic parameters of NLRP1 inflammasome inhibition. Apparent Km and Vmax were determined with or without GST-Bcl-2 or GST-Bcl-X_{L}. Aliquots containing 8.5 nM NLRP1 and 8.5 nM pro-Caspase-1 were incubated in Caspase-1 buffer for 30 min at 37° C. in the presence of activators (MDP-LD, ATP, and Mg²⁺) in a total volume of 10 μl. Reaction mixtures were subsequently assayed with various concentrations of Ac-WEHD-AMC substrate in 96-well plates. Km and Vmax were determined using a nonlinear regression method to fit the Michaelis-Menten (MM) equation: V=(Vmax-V₀)[S]/(Km+[S])+V₀, where V=initial catalytic rate, in nanomolar of AMC/min; [S]=substrate concentration in nanomolar or micromolar; V₀=limiting value of V without [S]; Vmax=limiting value of V at saturating [S]. Competitive inhibition was determined after linearization of the MM equation, using the double reciprocal Lineweaver-Burk equation: 1/V=Km/Vmax[S]+1/Vmax. Equilibrium constants of inhibition (Ki) were determined based on the competitive inhibition equation: Km_{I}=Km (1+[I]/Ki), where Km_{I}=Km in presence of inhibitor; I=concentration of inhibitor. IC50s for loop peptides were estimated using a fitting procedure and the sigmoidal curve: V=V₀+(Vmax−V0)/(1+10̂(Log IC50-[I]))n, where n=Hill slope. Fitting procedures were performed using PRISM software.

Electrophoresis. Samples were incubated with protein solubilizing solution (40 mM 6-aminocaproic acid, 20 mM Bis-tris) for 30 min at 4° C., mixed with loading buffer (1M 6-aminocaproic acid, 5% Serva blue G), and applied to acrylamide-bis gradient (4-20%) gels (Schagger et al., (1994) Anal Biochem 217, 220-30). Bands of gels corresponding to the entire lane were excised from top to the bottom, incubated 45 min at 25-27° C. in denaturating buffer (1% β-mercaptoethanol, 10% SDS), then washed twice in equilibrating buffer (50 mM Tris. 1% SDS). Bands from gels were set horizontally on a SDS-acrylamide gel, and proteins were separated by electrophoresis (second dimension), then stained with Sypro Ruby. Molecular weight markers included Thyroglobuline, Apoferritin, β-Amylase and BSA (Sigma).

Fluorescence polarization assays. NLRP1 or NLRP1ΔLRR were incubated with or without ligand in ice for 10 min, and then with 10 nM of Fluorescein-conjugated synthetic purified ATP in 20 mM HEPES pH 7.5, 150 mM NaCl, 0.1% CHAPS, 1 mM DTT for 5 min at 4° C. Fluorescence polarization was measured using an Analyst AD Assay Detection System (LJL Biosystem).

Reagents and antibodies. MDP-LD was obtained from Alexis. Ac-WEHD-AMC was purchased from Calbiochem. Antibodies targeting Caspase-1 p10 were obtained from Santa Cruz Biotechnology. Rhodamine-conjugated secondary anti-rabbit antibodies were purchased from Licor.

Statistical Analysis. Most data were presented as the mean±SD from at least three independent experiments. Statistical comparisons between different treatments were performed by unpaired t test, where p≦0.05 was considered statistically significant.

ii. Results

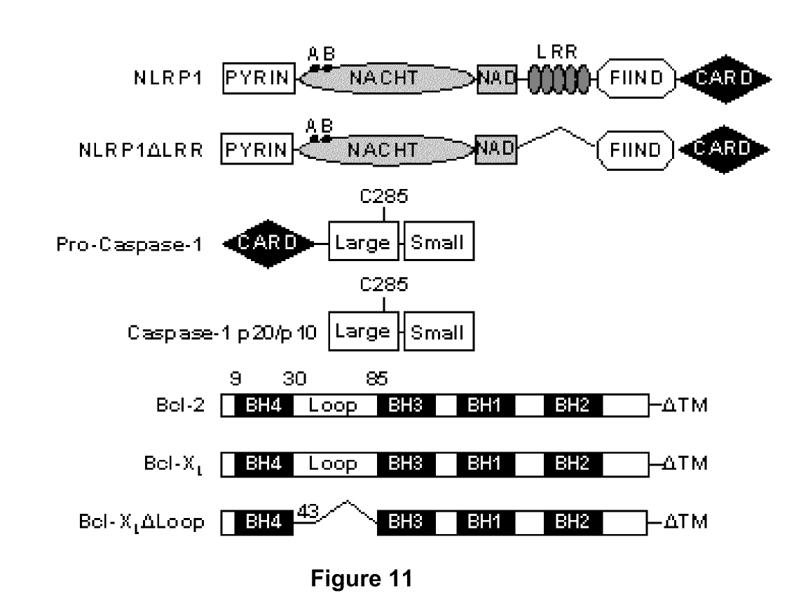

Bcl-2 and Bcl-X_{L} inhibit NLRP1-mediated Caspase-1 activation in vitro. Previously, the baculovirus expression system was used to produce and purify NLRP1 and pro-caspase-1 proteins and showed that the combination of MDP and ATP induces NLRP1 oligomerization and Caspase-1 activation (Faustin 2007). Using this in vitro reconstituted system, the effects of recombinant Bcl-2 and Bcl-XL proteins produced in bacteria were then tested. In addition, these proteins were contrasted with a mutant of Bcl-XL lacking a “loop” between the first and second a-helices, which previously was determined to be important for binding of Bcl-XL to NLRP1 in cells (Bruey, 2007 Cell) (FIG. 11). For these initial experiments, Bcl-2-family and various control proteins were added at excess relative to NLRP1, then MDP and ATP were added, and then Caspase-1 activity was measured using a fluorigenic peptide substrate.

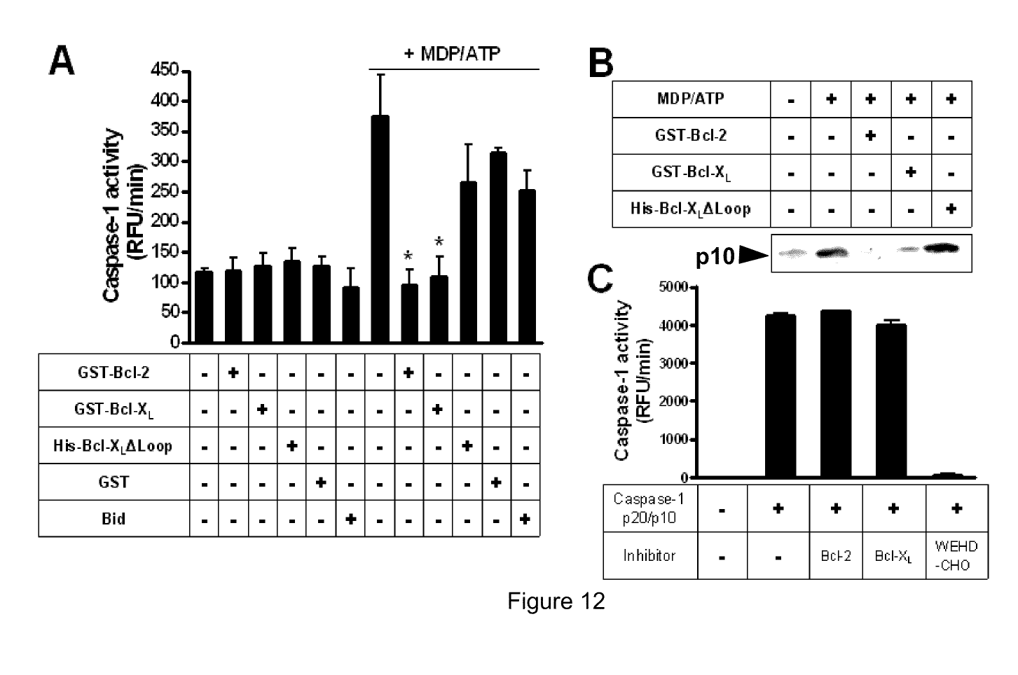

Consistent with the inhibition by Bcl-2 and Bcl-XL of NLRP1-mediated Caspase-1 activity, it was observed that MDP/ATP-induced proteolytic processing of pro-Caspase-1 to produce the cleaved p10 catalytic subunit (detected by immunoblot analysis of reactions) was also inhibited by Bcl-2 and Bcl-X_{L} (FIG. 12B). In contrast, Bcl-X_{L}ΔLoop failed to inhibit Caspase-1 cleavage.

To exclude a direct effect of Bcl-2 and Bcl-XL on Caspase-1, as opposed to NLRP1, the effects of these anti-apoptotic proteins were tested on fully active, proteolytically processed Caspase-1 comprised of p20 and p10 catalytic subunits purified from bacteria (FIG. 11). When added at excess relative to Caspase-1, neither Bcl-2 nor Bcl-XL suppressed Caspase-1 activity (FIG. 12C). In contrast, Caspase-1 was completely inhibited by a peptide inhibitor, acetyl-Tryptophanyl-Glutamyl-Histidinyl-Aspartyl-aldehyde (Ac-WEHD-CHO) (FIG. 12C).

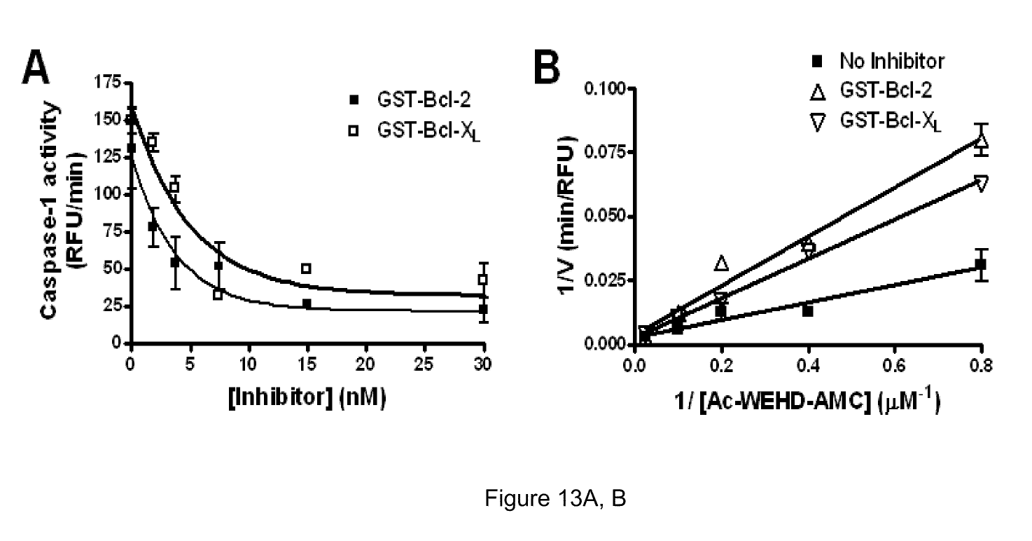

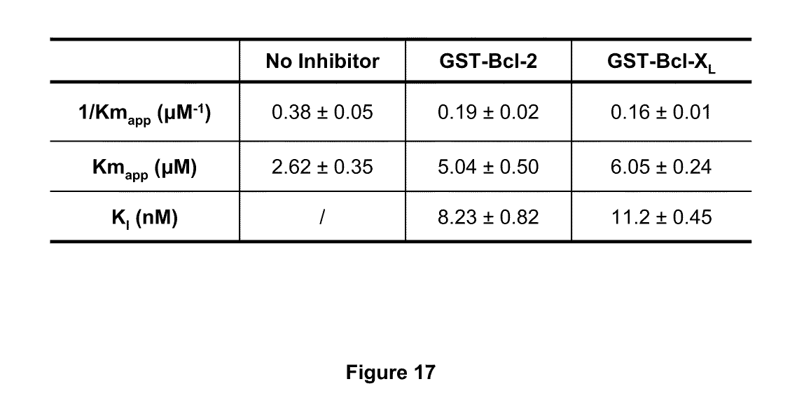

Bcl-2 and Bcl-X_{L} are competitive inhibitors of NLRP1. The mechanism of NLRP1 inhibition by Bcl-2 and Bcl-XL was next explored using classical enzyme kinetics analysis. First, the concentration-dependence of NLRP1 inhibition by Bcl-2 and Bcl-XL was determined, observing that suppression of NRLP1-induced Caspase-1 activity follows a hyperbolic curve to reach essentially complete inhibition at concentrations of Bcl-2 or Bcl-X_{L} that are two-times the concentration of NLRP1 (FIG. 13A). Second, a Lineweaver-Burke double reciprocal transformation of the data was performed (FIG. 13B), observing that addition of Bcl-2 or Bcl-X_{L} changed the x-axis intercept (−1/Km) but not the y-axis intercept (1/Vmax), thus suggesting a competitive mechanism of inhibition. Of note, the affinity (Km) of Caspase-1 for its substrate Ac-WEHD-AMC was reduced 2-fold in presence of Bcl-2 or Bcl-X_{L} compared to control (5.04±0.5 μM and 6.05±0.24 μM versus 2.62±0.35 μM; see FIG. 17). From these values, the inhibitory constants (Ki) for Bcl-2 and Bcl-XL suppression of NLRP1 were calculated as 8.23±0.82 nM and 11.2±0.45 nM, respectively (FIG. 17). Thus, Bcl-2 and Bcl-XL are potent (low nanomolar) inhibitors of NLRP1 with respect to its ability to activate Caspase-1.

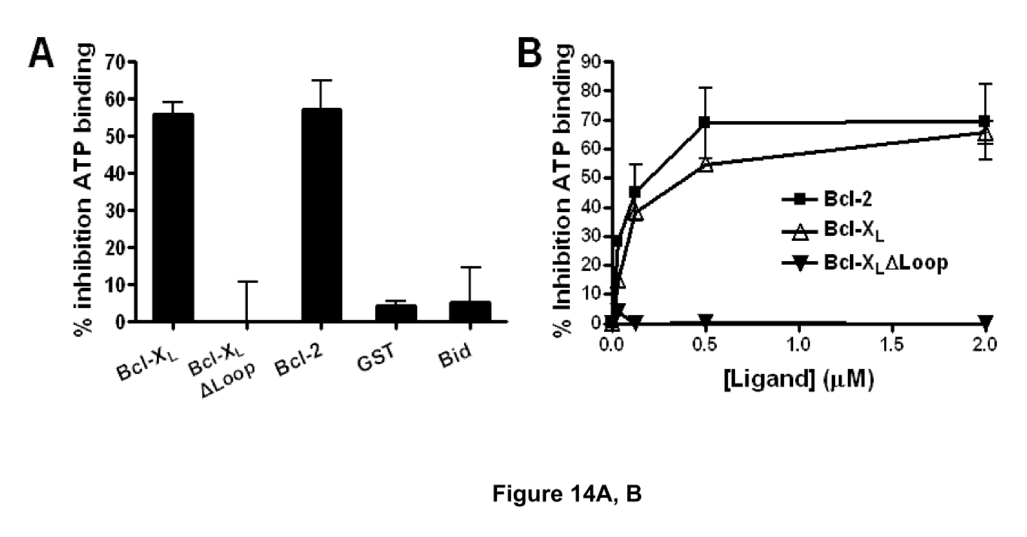

Bcl-2 and Bcl-X_{L} block ATP binding to NLRP1 and suppress oligomerization. Previously, using the reconstituted NLRP1 inflammasome (Faustin et al., (2007) Mol Cell 25, 713-24), it was determined that Caspase-1 is activated following a two step process, in which MDP binding to NLRP1 first induces a conformational change of the protein, which is secondarily followed by ATP binding to mediate NLRP1 oligomerization (Faustin 2007). Upon oligomerization of NLRP1, Caspase-1 monomers associate with NLRP1 oligomers, which results in protease activation, presumably via an induced dimerization mechanism. Among these molecular events, the binding of ATP to MDP-primed NLRP1 is readily measured by fluorescence polarization assay (FPA), using fluorescein isothiocyanate (FITC)-conjugated ATP (Faustin, 2007). FITC-ATP binding to recombinant NLPR1 was measured by FPA in presence and absence of Bcl-2-family proteins. Addition of Bcl-2 or Bcl-X_{L} at excess relative to NLRP1 reduced ATP binding by ˜60%, whereas control proteins GST, Bid, and Bcl-XL (Δloop) had negligible effects (FIG. 14A). Suppression of FITC-ATP binding to NLRP1 by Bcl-2 and Bcl-XL was concentration-dependent and saturable (FIG. 14B).

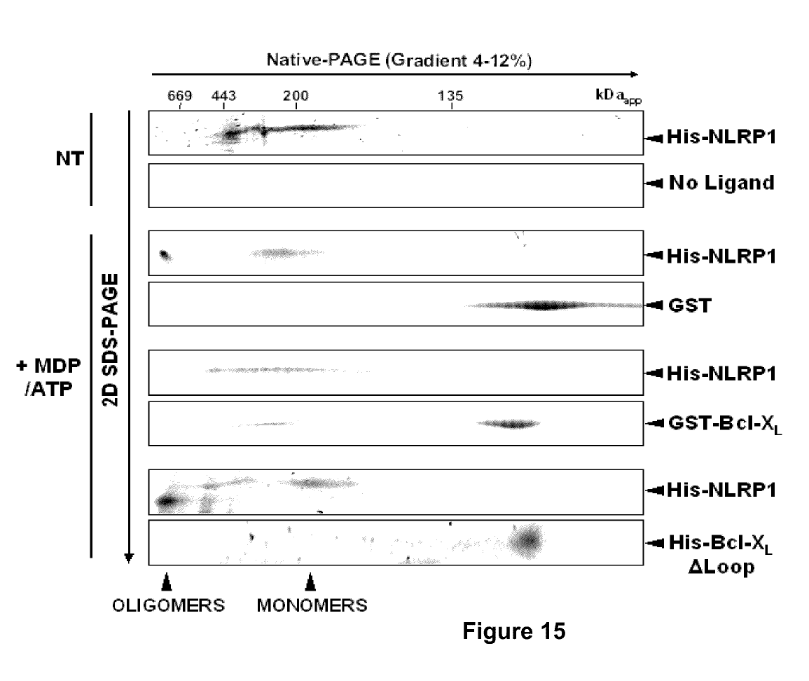

Since Bcl-2 and Bcl-X_{L} block ATP binding to NLRP1, we expected they would inhibit NLRP1 oligomerization. Oligomerization of NLRP1 induced by MDP and ATP can be monitored using 2D gel-electrophoresis analysis, in which the first dimension is non-denaturing and thus preserves macromolecular complexes during electrophoresis and the second dimension contains SDS for subsequent identification of proteins based on their known molecular mass (Faustin, 2007). For these experiments, NLRP1 monomers were incubated with various proteins (GST, GST-Bcl-XL, His6-Bcl-XLΔLoop) at excess, then MDP and ATP were added to induce oligomerization (FIG. 15). In the absence of other proteins, NLRP1 migrated in the ˜150-450 kDa range prior to addition of MDP/ATP. Addition of MDP/ATP caused much of the NLRP1 to shift to a larger complex (≧1 MDa)(FIG. 15), including material that failed to enter gels, and this oligomerization was not affected by addition of GST control protein (FIG. 15). Adding GST-Bcl-XL suppressed oligomerization of NLRP1. Moreover, GST-Bcl-XL protein was found co-migrating with non-oligomerized NLRP1 (range ˜150-450 kDa), consistent with the ability of Bcl-XL to bind NLRP1. In contrast, Bcl-X_{L}ΔLoop did not inhibit formation of NLRP1 oligomers, and this protein did not co-migrate with non-oligomerized NLRP1 (FIG. 15). All together, these data indicate that Bcl-X_{L} interacts with unoligomerized NLRP1 to inhibit ATP binding and suppress oligomerization.

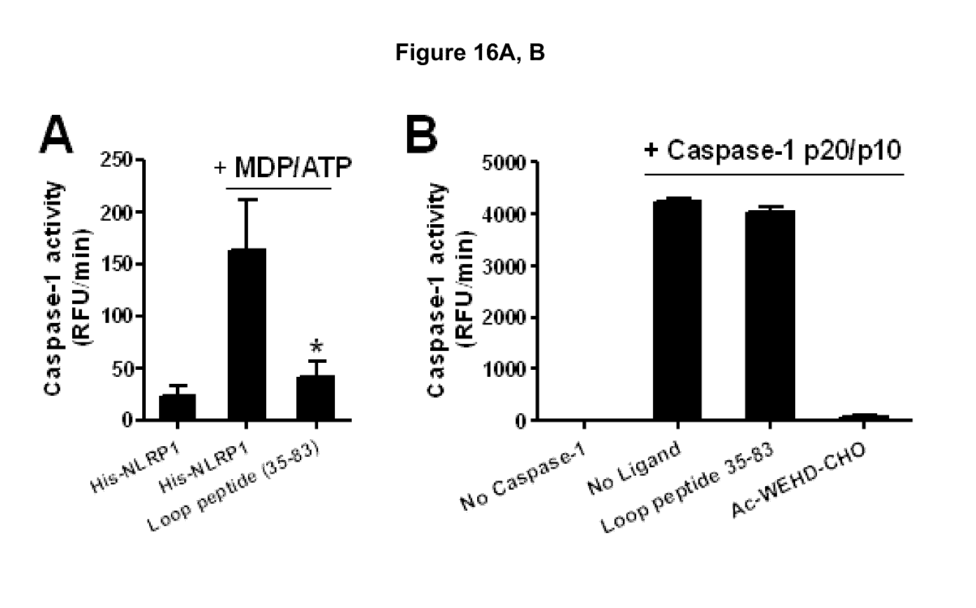

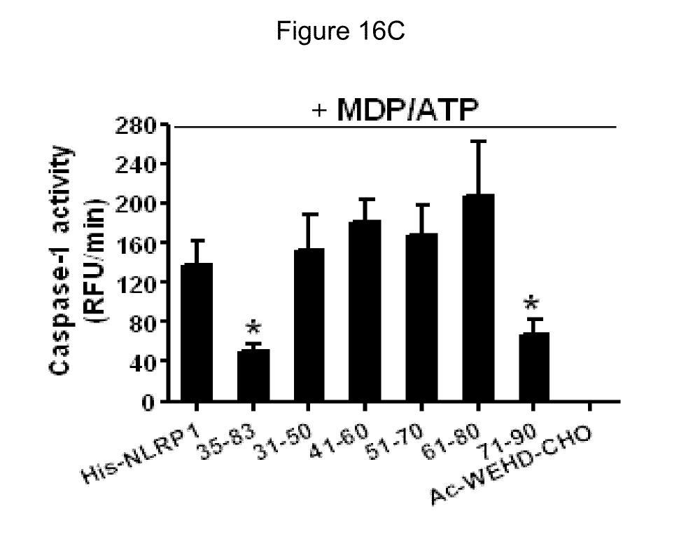

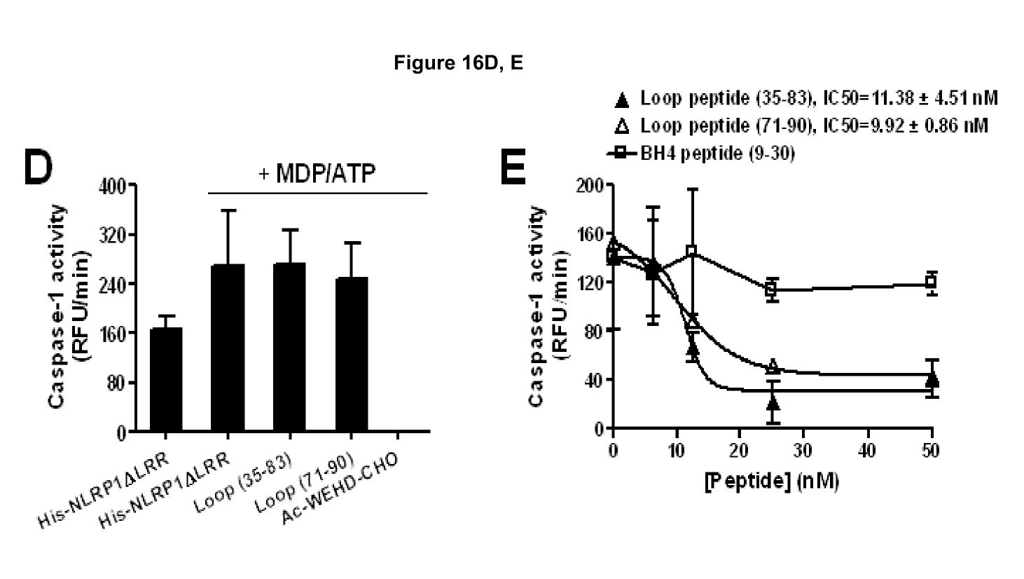

The loop region of Bcl-2 is sufficient to inhibit NLRP1. It was previously determined that the “loop” domains of Bcl-2 and Bcl-XL are required for binding to and suppression of NLRP1 in cells (Bruey, 2007 Cell). While necessary, it was unknown whether the loop region is sufficient for suppressing NLRP1. To study the effect of the loop domain in isolation, peptides corresponding to the loop region and other segments of Bcl-2 (FIG. 11) were chemically synthesized and tested their effects in vitro on the reconstituted NLRP1 inflammasome when added at excess relative to NLRP1. Addition of Bcl-2 loop peptide (residues 35-83) inhibited NLRP1-mediated Caspase-1 activation to nearly baseline levels (FIG. 16A). In contrast, this 35-83 Bcl-2 loop peptide did not directly inhibit Caspase-1, whereas the peptidyl inhibitor Ac-WEHD-CHO potently suppressed the activity of this protease (FIG. 16B).

To characterize the sequence of the Bcl-2 loop responsible for inhibiting NLRP1, a series of overlapping 20′mer peptides corresponding to the 35-83 region were synthesized and tested their effects on NLRP1-mediated Caspase-1 activation. These experiments showed that a peptide corresponding to residues 71-90 of Bcl-2 is sufficient to potently inhibit NLRP1, whereas other 20′mer peptides corresponding to 31-50, 41-60, 51-70, and 61-80 are inactive (FIG. 16C). The effects of the full-loop peptide (35-83) and the active segment (71-90) were specific, as determined by experiments using a NLRP1 mutant lacking the LRRs. Unlike full-length NLRP1, the ALRR mutant is constitutively active (not requiring MDP) and does not bind Bcl-2 or Bcl-XL (Bruey, 2007 Cell). As shown in FIG. 16D, neither the 35-83 nor the 71-90 loop peptides suppressed Caspase-1 activation by NLRP1ΔLRR, whereas Ac-WEHD-CHO peptide completely neutralized Caspase-1 activity. Finally, the concentration-dependence of NLRP1 inhibition by the 35-83 and 71-90 loop peptides were compared. Both peptides demonstrated similar concentration-dependent inhibition, with sigmoidal curves, reducing Caspaes-1 activity to background levels (FIG. 16E). The deduced IC50 values (concentrations to inhibit NLRP1-induced Caspase-1 activity by 50%) for the full-length 35-83 and 71-90 loop peptides were 11.38±4.51 nM and 9.92±0.86 nM, respectively. In contrast, a control peptide corresponding to residues 9-30 (BH4 domain) had negligible activity in this assay.

These results indicate that residues 71-90 of Bcl-2 loop region are sufficient to inhibit NLRP1.

iii. Discussion

It has been demonstrated here that Bcl-2 and Bcl-X_{L} directly and potently inhibit NLRP1. These studies were performed using purified recombinant proteins, thus eliminating the contributions of other factors, and unambiguously demonstrating a direct inhibitory effect of Bcl-2 and Bcl-XL on NLRP1. The Ki's for Bcl-2 and Bcl-XL were ˜10 nM, suggesting physiologically relevant suppression. Bcl-2 and Bcl-XL suppress their target (NLRP1) with similar potency to XIAP inhibiting Caspase-9. Furthermore, the concentration-dependent suppression of NLRP1 by Bcl-2 and Bcl-XL indicates a 1:1 molar complex. The mechanism of Bcl-2- and Bcl-XL-mediated suppression of NLRP1 is competitive, as shown by Lineweaver-Burke analysis. Because competitive inhibitors reduce the affinity of enzymes for their substrates, this result indicates that in the presence of Bcl-2-family proteins, the active site of Caspase-1 does not form, thus effectively impairing the affinity (Km) of Caspase-1 for its substrate (Ac-WEHD-AMC). In this regard, it was observed that Bcl-2 and Bcl-XL inhibit ATP binding by MDP-activated NLRP1, an event required for oligomerization of NLRP1, and it was documented by non-denaturing gel-electrophoresis experiments that Bcl-XL suppresses oligomerization of NLRP1. Thus, it is concluded that the active site of Caspase-1 does not form because Bcl-2 and Bcl-XL prevent NLRP1 oligomerization, and thus Caspase-1 molecules are not brought into close apposition for activation. This conclusion is consist with evidence that initiator Caspases such as Caspase-8 and -9 require assisted dimerization to induce conformational changes that establish the functionally active site of these enzymes.

Because Bcl-2 and Bcl-XL inhibit NLRP1 even when its activating ligand MDP is provided, it is likely that two events are required to achieve NLRP1 activation in those cell-types where Bcl-2 or Bcl-XL is playing an inhibitory role. First, repression by Bcl-2 and Bcl-XL must be relieved. Second, once freed of Bcl-2 and Bcl-XL, NLRP1 must be activated by MDP or functionally equivalent ligands. How Bcl-2 and Bcl-XL are dissociated from NLRP1 in cellular contexts remains to be clarified. Previous domain mapping experiments showed that the NACHT and LRRs of NLRP1 are required for Bcl-XL binding (Bruey, 2007 Cell). Thus, post-translation modifications, changes in protein conformation, and interaction of other proteins or of small non-protein ligands with the NACHT-LRR region of NRLP1 may free this NLR-family member from Bcl-2 and Bcl-XL. Alternatively, control of dissociation may occur at the level of Bcl-2 and Bcl-XL, affecting directly or indirectly the interacting loop domain. Regardless, requirement for two steps removal of repression (Bcl-2/Bcl-XL) and addition of activating ligand (MDP)—would help to ensure that NLRP1 activation occurs only in appropriate cellular contexts, thus reducing chances for dysregulated production of IL-1b and other pro-inflammatory cytokines activated by Caspase-1.

Prior studies in which the loop regions of Bcl-2 and Bcl-X_{L} were shown to be required for association with and suppression of NLRP1 in cells (Bruey, 2007 Cell) were extended by showing here that deletion of the loop from purified recombinant Bcl-XL eliminates suppression of NLRP1 in vitro. Bcl-XLΔloop also failed to inhibit ATP binding and oligomerization of MDP-activated NLRP1, unlike intact Bcl-XL. Moreover, it was also demonstrated that the loop of Bcl-2 is sufficient to inhibit NLRP1, based on experiments using synthetic peptides corresponding to this region of Bcl-2. Previously, it was unclear whether the loop domain directly inhibits NLRP1, versus the possibility that it affects the overall conformation of Bcl-2 and Bcl-XL in a manner such that the loop is required to generate conformational states that are competent to bind NLRP1. The data provided here indicate that the loop directly inhibits NLRP1. Moreover, it was observed that a synthetic 20′mer loop peptide corresponding to residues 71-90 is sufficient to suppress NLRP1-mediated Caspase-1 activation. This peptide binds to NLRP1 with a high affinity (IC50˜10 nM), which is comparable to full-length loop peptide 35-83 (IC50˜12 nM), and to the full-length Bcl-2 and Bcl-X_{L} proteins (Ki-10 nM). Thus, the loop contains a peptidyl-ligand for NLRP1 that suppresses its activation. In this regard, the loop regions of Bcl-2 and Bcl-X_{L} are unstructured in the available 3D structures of these proteins, but when bound to NLRP1, segments of the loop may develop secondary structure or constrained structures that adapt to the relevant interacting surface of NLRP1. Interestingly, the loops of Bcl-2 and Bcl-XL are subject to post-translational modifications, which to date include phosphorylation, deamidation, and proteolytic cleavage (Cheng et al., (1997) Science 278, 1966-8; Yamamoto et al., (1999) Mol Cell Biol 19, 8469; Ojala et al., (2000) Nat Cell Biol 2, 819-25). Such modifications, for example, might contribute to mechanisms for releasing NLRP1 to allow subsequent Caspase-1 activation. Also meriting note, somatic mutations have been reported in the loop region of Bcl-2 in lymphomas where the gene that encodes this anti-apoptotic protein is involved in chromosomal translocations with the immunoglobulin heavy-chain gene locus.

The demonstration that a peptide derived from the loop of Bcl-2 is sufficient to recapitulate the inhibitory activity of the intact protein indicates that the Bcl-2 loop region and peptides comprising the minimal NLR ligand can be used as the basis of therapeutic cell-permeable peptides for suppressing NLRP1 activity in inflammatory diseases and of generating high-throughput screening (HTS) assays using these peptides for identification of chemical compounds that occupy the same peptide binding site on NLRP1 and that mimic the inhibitory activity of the Bcl-2 loop peptide. Such agents will be useful for preventing NLRP1-mediated activation of pro-inflammatory cytokines and for suppressing NLRP1-induced cell death in pathological conditions.

### 2. EXAMPLE 2

### An Amino Acid Sequence in the N-Terminal 48 Amino Acids Of F1L Vaccinia Virus Protein Inhibits ATP Binding and Oligomerization in NLRP1 Inflammasome

The F1L peptide has been tested using the in-vitro reconstitution of NLRP1 inflammasome (see Example 1). Using this system defined in the laboratory, it has been demonstrated that F1L peptide (22-47) inhibits NLRP1-mediated caspase-1 activation by monitoring the cleavage of a fluorogenic substrate of caspase-1. Addition of F1L peptide suppresses the ATP binding to NLRP1 by using Fluorescent Polarization Assay (FPA). F1L interacts directly to NLRP1 by using FPA with a Fluoroscein-conjugated F1L peptide (FAM-F1L 22-47). This binding is displaced by the addition of cold peptide. For each experiment, the use of another vaccinia virus-encoded protein N1L has been used as negative control, since we evidenced previously that N1L does not bind to NLRP1 in HEK293T cells after overexpression by using co-immunoprecipitation assays.

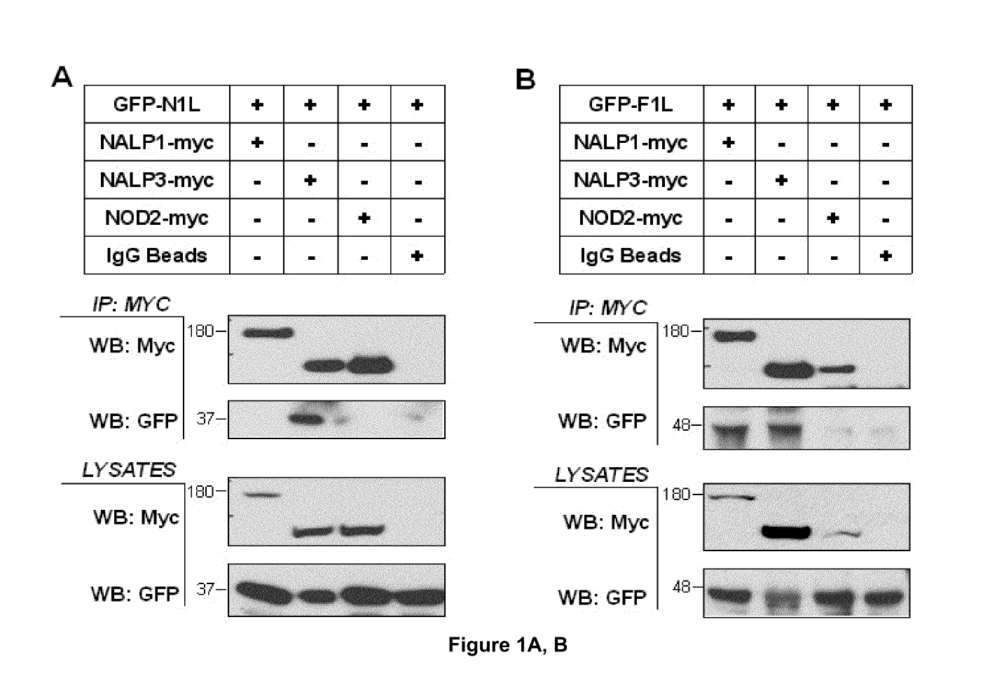

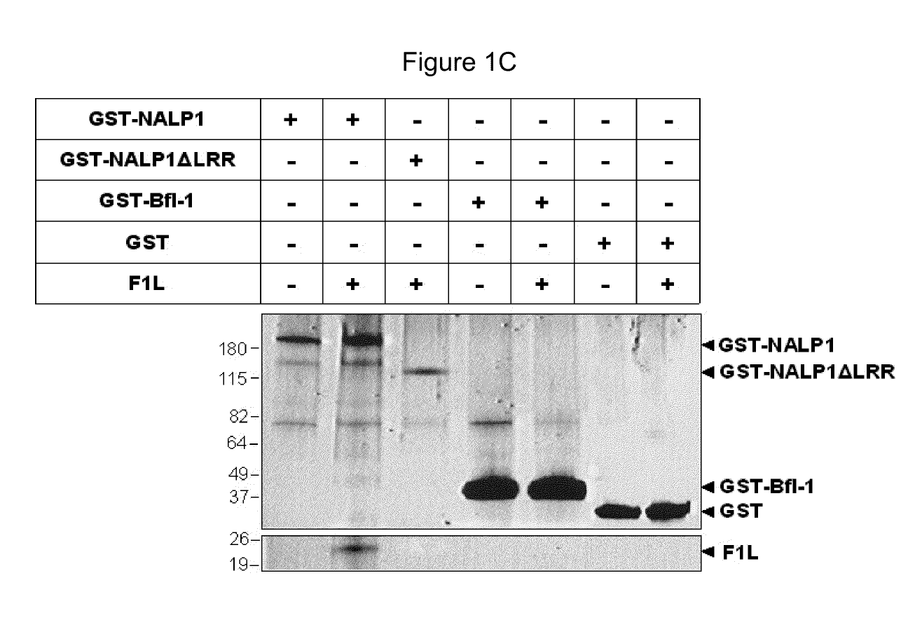

FIG. 1 shows data indicating that viral Bcl-2 homolog F1L of vaccinia virus bind NLRs. HEK293T cells were transiently co-transfected with plasmids encoding myc-tagged NLR full-length proteins and either (A) GFP-tagged N1L or (B) GFP-tagged F1L. For co-immunoprecipitations, 5×105-1×106 cells were lysed in isotonic lysis buffer (150 mM NaCl, 20 mM Tris/HCl (pH 7.4), 0.3% NP-40, 2 mM NaF, 1 mM DTT, 1 mM PMSF, and 1× protease inhibitor mix (Roche). Clarified lysates were subjected to immunoprecipitation (IP) using agarose conjugated anti-c-Myc. After incubation at 4° C. for 12 hr, immune-complexes were washed 3 times in lysis buffer, separated by SDS/PAGE and analyzed by immunoblotting (“WB”) using various antibodies as indicated in conjunction with ECL detection system (Amersham-Pharmacia). Where indicated, cell lysates (10% volume) were run along side immune-complexes. Alternatively, lysates were directly analyzed by immunoblotting after normalization for total protein content. (C) Baculovirus-expressed recombinant proteins GST-NLRP1, GST-NLRP1ALRR, GST-Bfl-1, or GST (5 μg) were mixed with or without purified F1L (10 μg) in 20 mM HEPES-KOH, [pH 7.5], 10 mM KCl, 1 mM DTT in a final volume of 50 μl for 30 minutes on ice, followed by incubation overnight with Glutathione-Sepharose at 4° C. GST-purified complexes were isolated after centrifugation and eluted with SDS-loading buffer, analyzed by SDS-PAGE, and stained by Sypro Ruby.

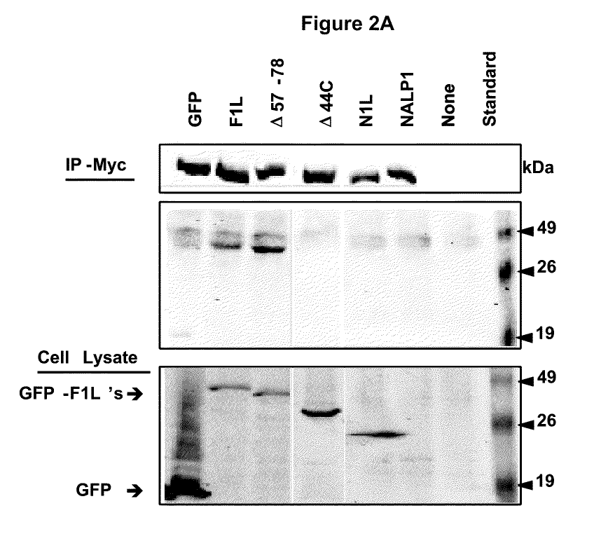

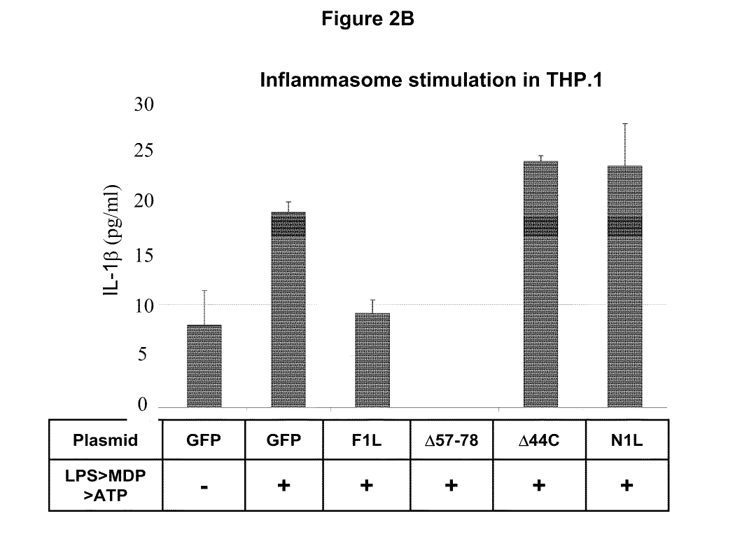

FIG. 2 shows data indicating that F1L (residues 1-44) can bind NLRP1 and that F1L lacking residues 1-44 cannot bind. (A) HEK293T cells were transiently co-transfected with plasmids encoding myc-tagged NLRP1 full-length proteins and either GFP, GFP-F1L, GFP-F1LΔ57-78, GFP-F1LΔ44-C, or GFP-N1L. For co-immunoprecipitations, 5×105-1×106 cells were lysed in isotonic lysis buffer (150 mM NaCl, 20 mM Tris/HCl (pH 7.4), 0.3% NP-40, 2 mM NaF, 1 mM DTT, 1 mM PMSF, and 1× protease inhibitor mix (Roche). Clarified lysates were subjected to immunoprecipitation (IP) using agarose conjugated anti-c-Myc. After incubation at 4° C. for 12 hr, immune-complexes were washed 3 times in lysis buffer, separated by SDS/PAGE and analyzed by immunoblotting (“WB”) using various antibodies as indicated. Where indicated, cell lysates (10% volume) were run along side immune-complexes. Alternatively, lysates were directly analyzed by immunoblotting after normalization for total protein content. (B) F1L (residues 1-44) can inhibit MDP-inducible IL-1β production in macrophages and that F1L lacking residues 1-44 does not inhibit. THP.1 monocytes were electroporated with the above plasmids (0.5 μg total) using the AMEXA system. Cells were differentiated by TPA stimulation over night followed by LPS (10 pg/ml) treatment. Cells were then treated with MDP-LD for 4 hr before pulsing with 2.5 mM ATP for 20 min. Supernatants were collected 3 hrs later and IL-1β secretion was measured by ELISA (mean±SD; n=3).

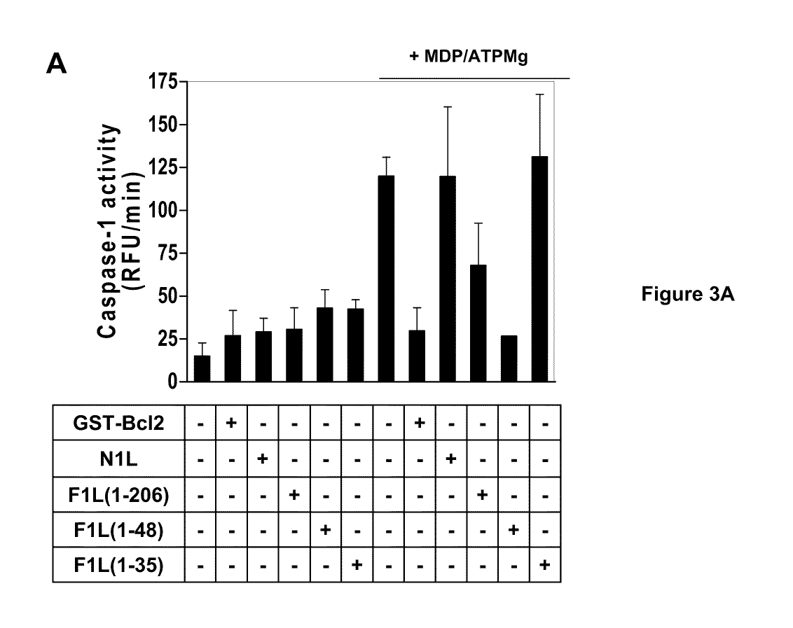

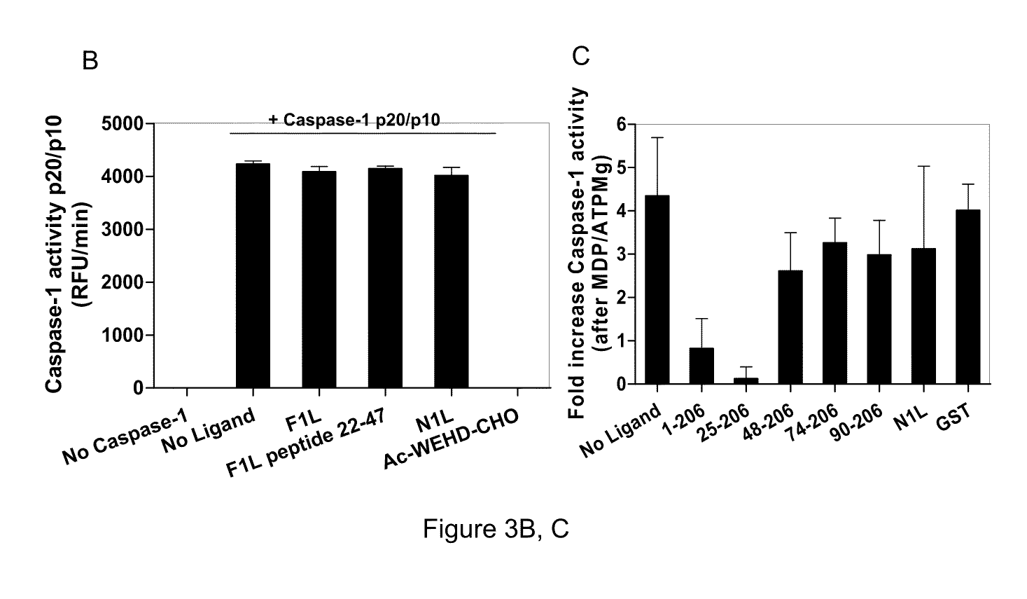

FIG. 3 shows data indicating that recombinant F1L (residues 1-48), but not N1L, suppresses the in vitro reconstituted NLRP1 inflammasome. (A) Reactions contained His-NLRP1 (8.5 nM), pro-caspase-1 (8.5 nM), 0.25 mM ATP, 0.5 mM Mg2+, 0.1 μg/ml MDP and GST-Bcl-2 or N1L, or various constructs of recombinant F1L (17 nM). Caspase-1 activity was measured after 60 minutes by hydrolysis of Ac-WEHD-AFC substrate (20 μM), expressing data as mean±SD, n=3. (B) Reactions contained His-NLRP1 (8.5 nM), pro-caspase-1 (8.5 nM), 0.25 mM ATP, 0.5 mM Mg2+, 0.1 μg/ml MDP and various constructs of recombinant F1L or N1L (17 nM). Caspase-1 activity was measured after 60 minutes by hydrolysis of Ac-WEHD-AMC substrate (20 μM), expressing data as mean±SD, n=3.

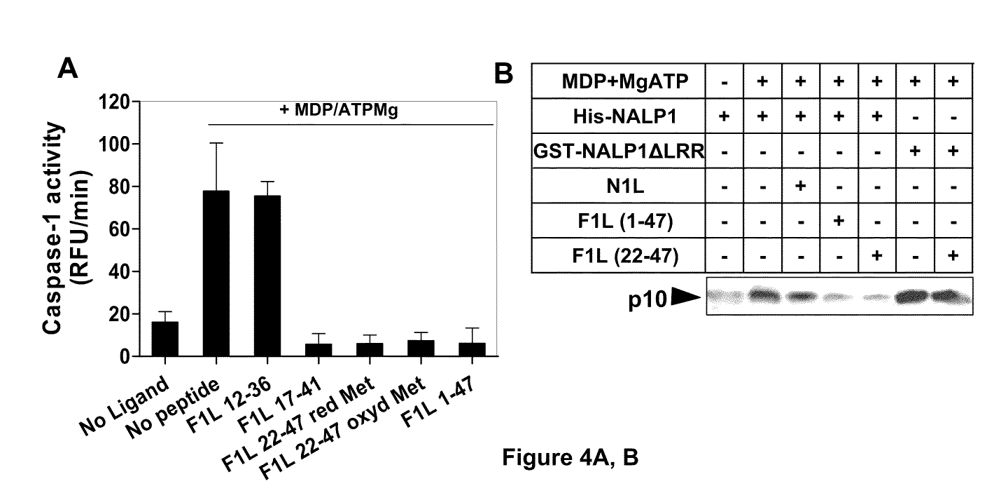

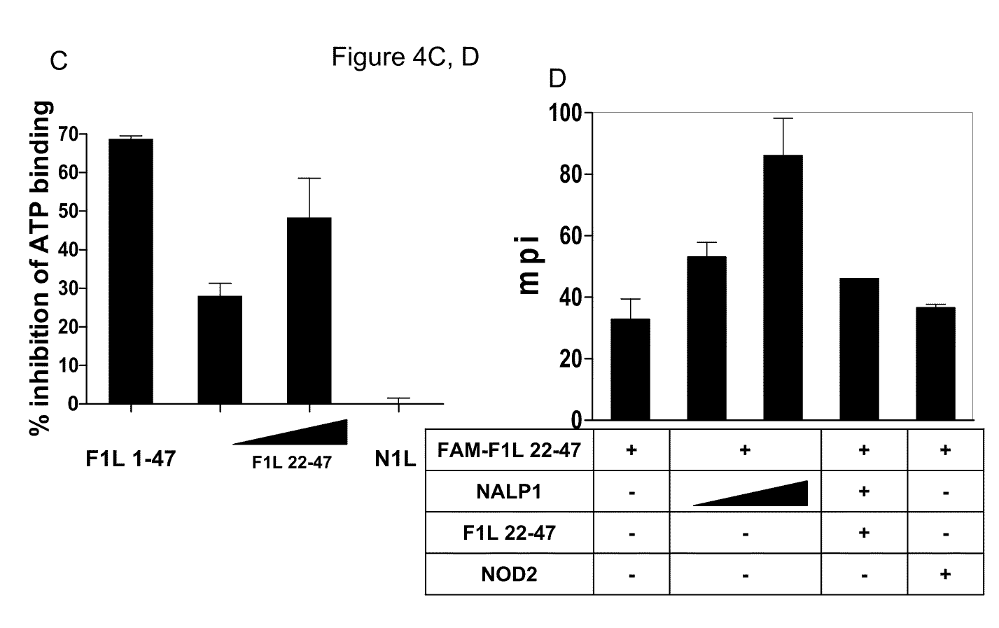

FIG. 4 shows data that synthetic F1L peptide (22-47) inhibits the ATP binding to NLRP1. (A) Reactions contained His-NLRP1 (8.5 nM), pro-caspase-1 (8.5 nM), 0.25 mM ATP, 0.5 mM Mg2+, 0.1 μg/ml MDP and various synthetic peptides of F1L (17 nM). Caspase-1 activity was measured after 60 minutes by hydrolysis of Ac-WEHD-AMC substrate (20 μM), expressing data as mean±SD, n=3. Caspase-1 activity was measured after 60 minutes by hydrolysis of Ac-WEHD-AMC substrate (20 μM), expressing data as mean±SD, n=3. (B) Reactions contained His-NLRP1 or GST-NLRP1ALRR (0.1 μM), pro-caspase-1 (0.1 μM), 1 mM ATP, 1 mM Mg2+, 10 μg/ml MDP and recombinant F1L 1-47 or synthetic F1L 22-47, or recombinant N1L (0.4 μM) were incubated 30 min at 37° C. Proteins were separated by SDS/PAGE and analyzed by immunoblotting using anti-p10 caspase-1 antibodies. (C) His-NLRP1 (0.125 μM) was incubated for 5 min in ice in the presence of F1L1-47 (2 μM), F1L 22-47 (0.5 or 2 μM), or N1L (2 μM). The mixture was then incubated for additional 5 min in ice with 1 μM MDP-LD, FL-conjugated ATP analog (10 nM) and Mg2+ (0.5 mM). ATP binding was analyzed by FPA (n=3 milliPolars [mP]), and the percentage of inhibition was determined vs NALP1 incubated only with MDP-LD (mean±SD). (D) His-NLRP1 (0.5 or 2 μM) or His-NOD2 (2 μM) was incubated for 5 min in ice with or without F1L 22-47 (0.1 μM). The mixture was then incubated for additional 5 min in ice with FAM-conjugated F1L 22-47 peptide (0.1 μM). Peptide binding was analyzed by FPA (n=3 milliPolars NAPA mean±SD.

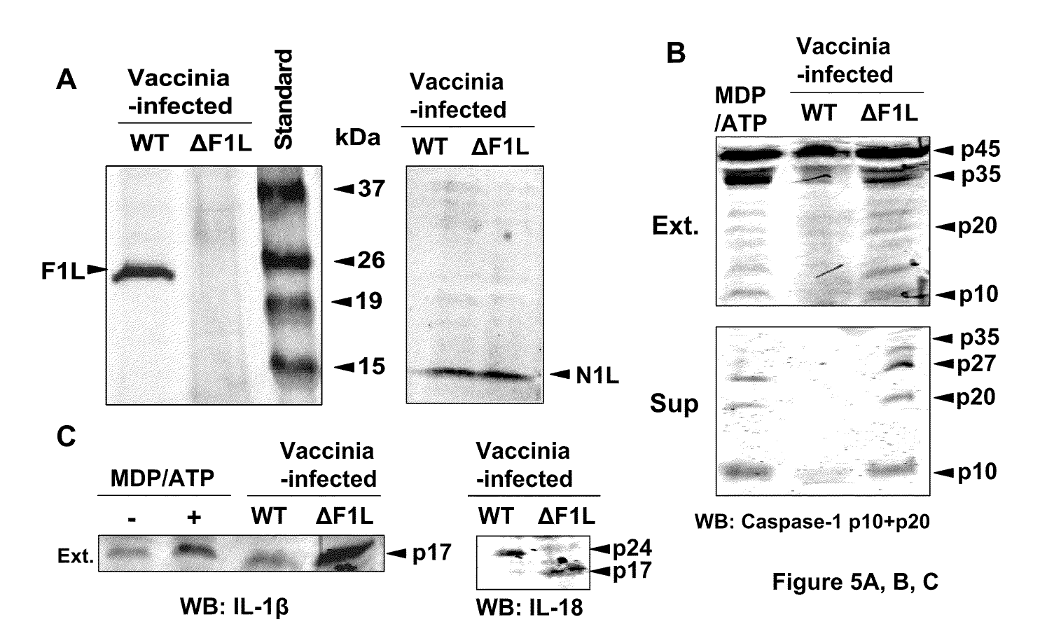

FIG. 5 show data indicating that F1L inhibits caspase-1 cleavage in vaccinia-infected macrophages. TPA-differentiated THP-1 cells were infected during 24 h (MOI=1) with wild-type (WT) vaccinia virus, or virus deleted in the gene encoding F1L. Culture supernatants were filtered using an Amicon Ultra (100 kDa cut-off). Proteins contained in eluates were precipitated by Trichloroacetic acid (TCA) and washed twice with acetone. Dry pellets were resuspended in 2× Laemmli buffer, normalized by the amount of protein, and loaded on top of a 15% SDS-polyacrylamide gel. (B) Cleaved fragments of caspase-1 were detected by western-blot using anti-p10 and p-20 antibodies (Santa Cruz). (C) Mature IL-1b (p17) and IL-18 cytokine were detected in the cell lysate. As positive control for caspase-1 cleavage, macrophages were primed 12 h with LPS (50 ng/ml), incubated for 4 h with MDP (2 μg/ml), followed by ATP (2.5 mM) for 20 min.

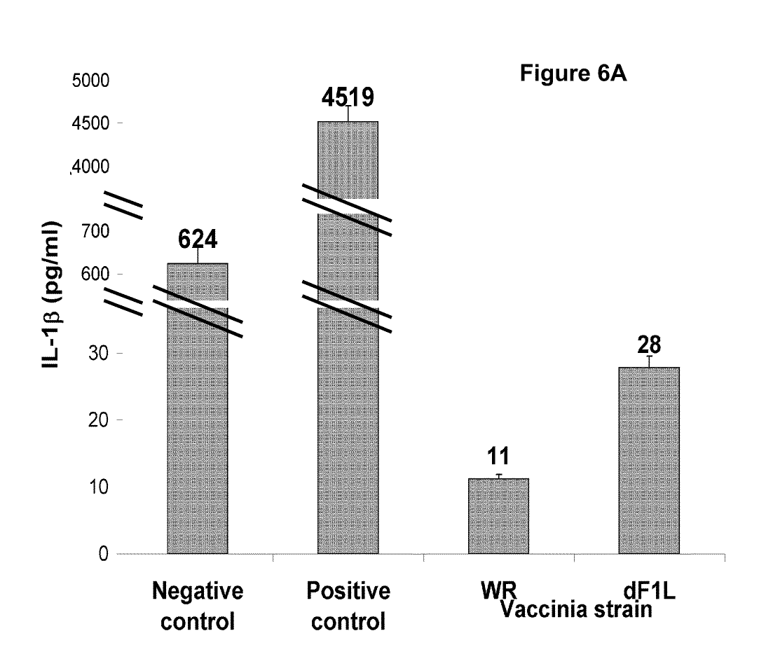

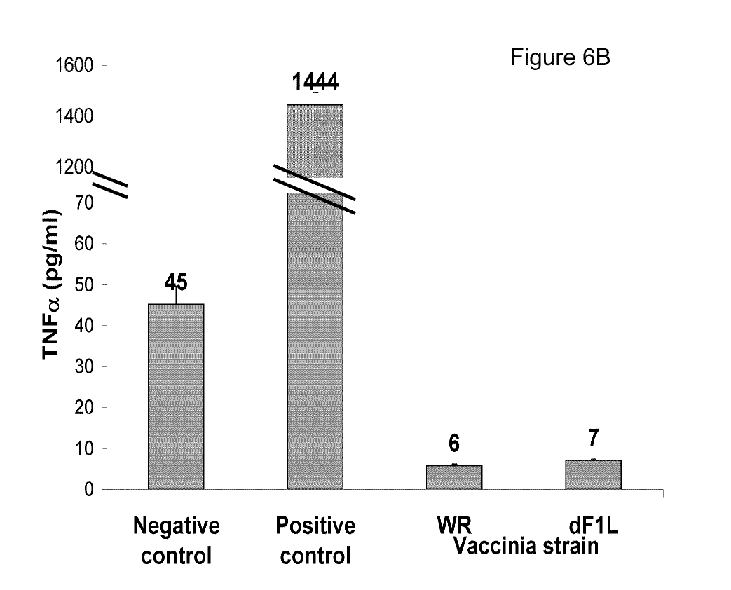

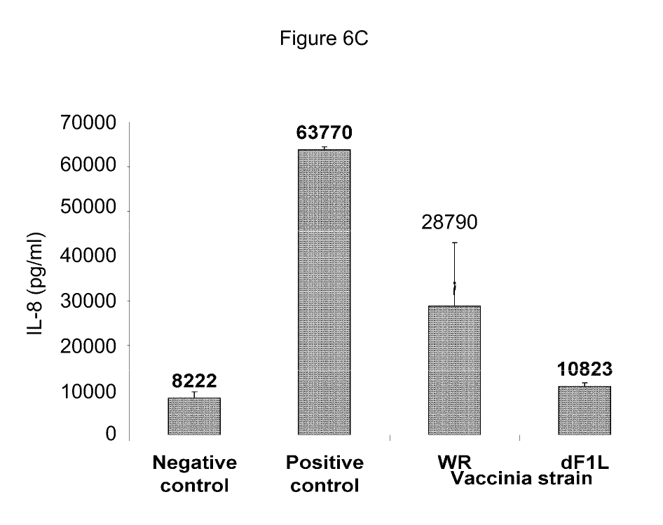

FIG. 6 shows data indicating that F1L inhibits proIL-1β processing in vaccinia-infected macrophages. TPA-differentiated THP-1 macrophages were infected (MOI=1) with wild-type (WT) vaccinia virus or mutant ΔF1L virus. Supernatants were collected 24 hrs later and concentrated (10×) using spin columns (Vivaspin 15R, 5000 MWCO, Sartorius AG). Cytokines secretion (A) IL-8, (B) TNFα and (C) IL-1β were measured by ELISA (e-Bioscience) (mean±SD; n=3). As positive control for Cytokines secretion, macrophages were primed 12 h with LPS (50 ng/ml), incubated for 4 h with MDP (2 μg/ml), followed by ATP (2.5 mM) for 20 min.

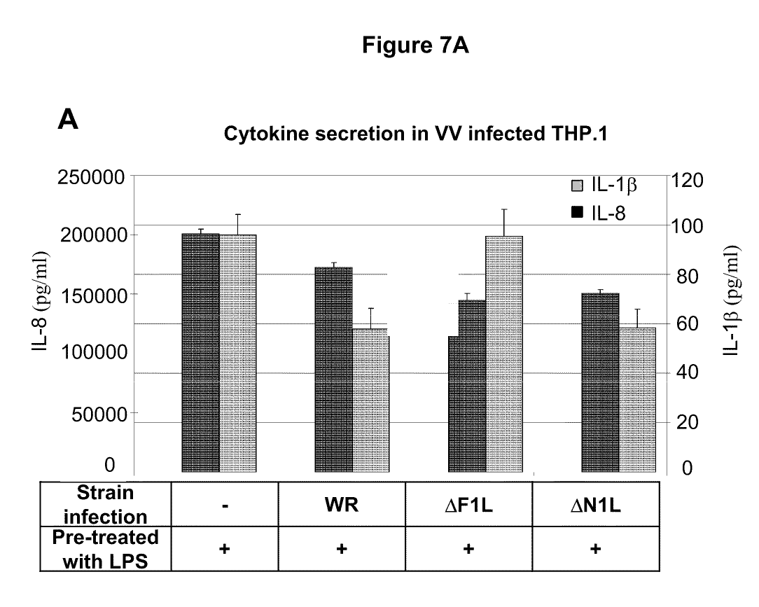

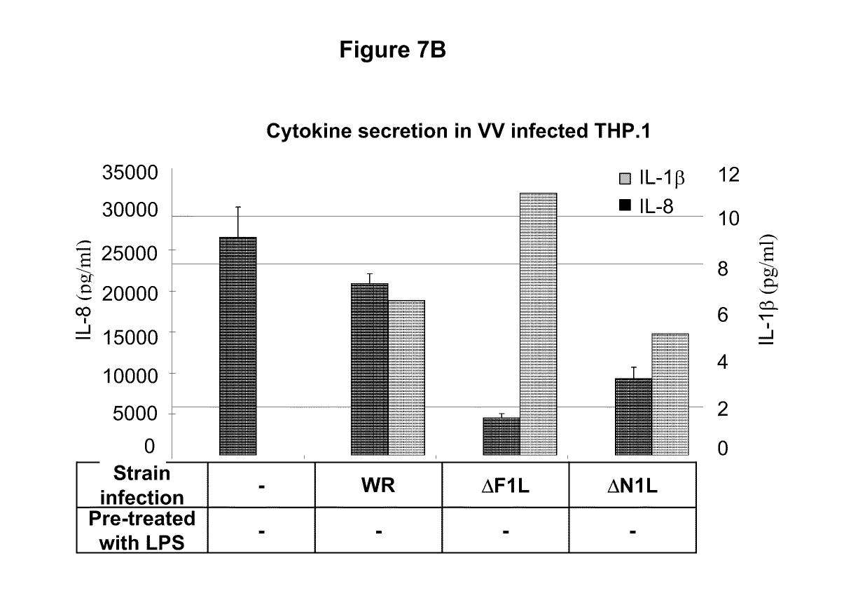

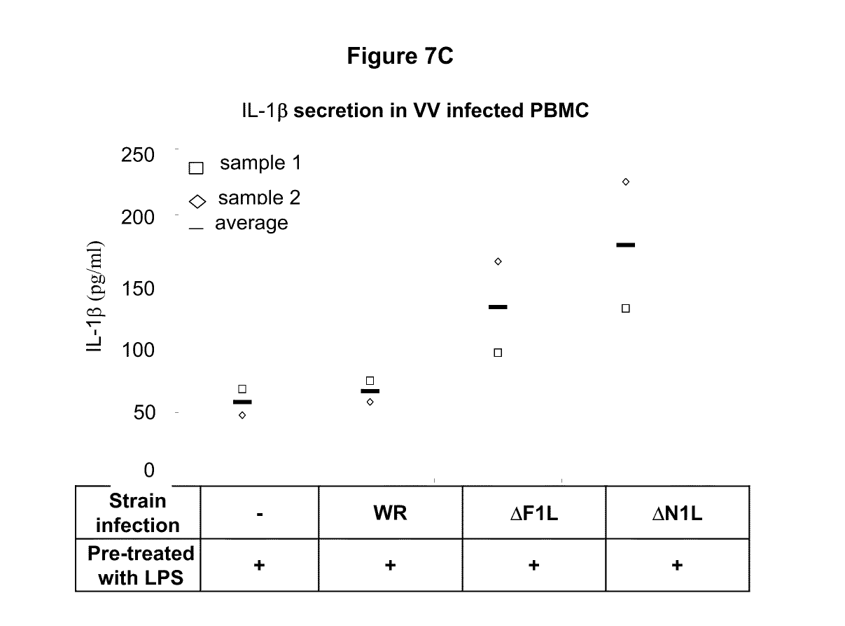

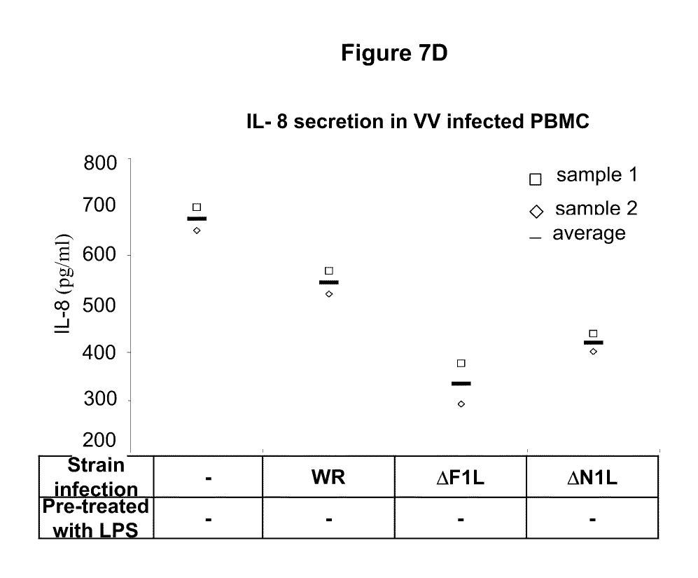

FIG. 7 shows data indicating that F1L inhibits proIL-1β processing in vaccinia-infected macrophages and PBMC. (A-B) TPA-differentiated THP-1 macrophages were either primed with LPS (10 pg/ml) or not. After 18 hr cells were infected (MOI=1) with wild-type vaccinia virus (WR) or mutant virus (ΔF1L or DN1L). Supernatants were collected 24 hrs later and concentrated and cytokines secretion IL-8 and IL-1β were measured by ELISA (e-Bioscience) (mean±SD; n=3). (C-D) F1L inhibits proIL-1β processing in vaccinia-infected PBMC. PBMC were collected using Ficol and left to grow over-night (n=2). PBMC were then primed with LPS (10 pg/ml) for 18 hrs prior to Vaccinia virus infection. Cytokines secretion IL-8 and IL-1β were measured by ELISA (e-Bioscience) (mean±SD; n=3).

NLRP1 regulates DF1L vaccinia virus-inducible IL-1β production in THP.1 macrophages. THP.1 monocytes were infected with various lenti virus, targeting different sites within the NLRP1 mRNA (NLRP1 592 or 1437). TPA-differentiated THP-1 cells were infected (MOI=1) with wild-type (WT) vaccinia virus or mutant virus (ΔF1L or DN1L). Supernatants were collected 24 hrs later and concentrated and cytokines secretion IL-8 and IL-113 were measured by ELISA (e-Bioscience) (mean±SD; n=3). NLRP1 knockdown evaluation in TPA-differentiated THP.1 cells was preformed by measuring the mRNA expression levels (Q-PCR) or by measuring IL-1b secretion after LPS (50 ng/ml) 18 hr, MDP (5 μg/ml) 4 hr, ATP (2.5 mM) for 20 mM simulation.

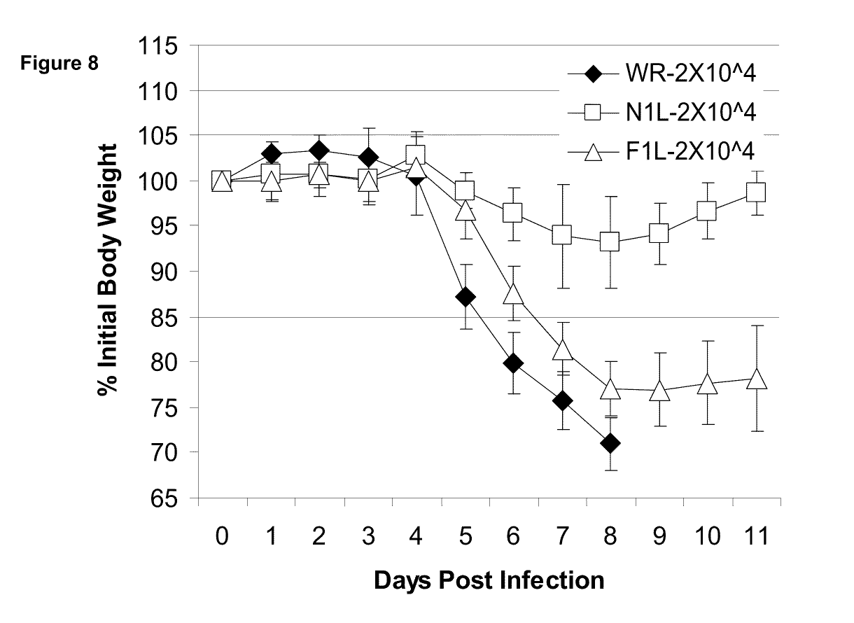

FIG. 8 shows the in-vivo phenotype of vaccinia virus. Balb/c mice were i.n. infected with (2×10⁴ PFU per mice) with wild-type vaccinia virus (WR) or mutant virus (ΔF1L or DN1L). Body weight was monitored during 11 days post infection. In this model mice which lost more then 30% of their initial body weight were dieing, and there for sacrificed.

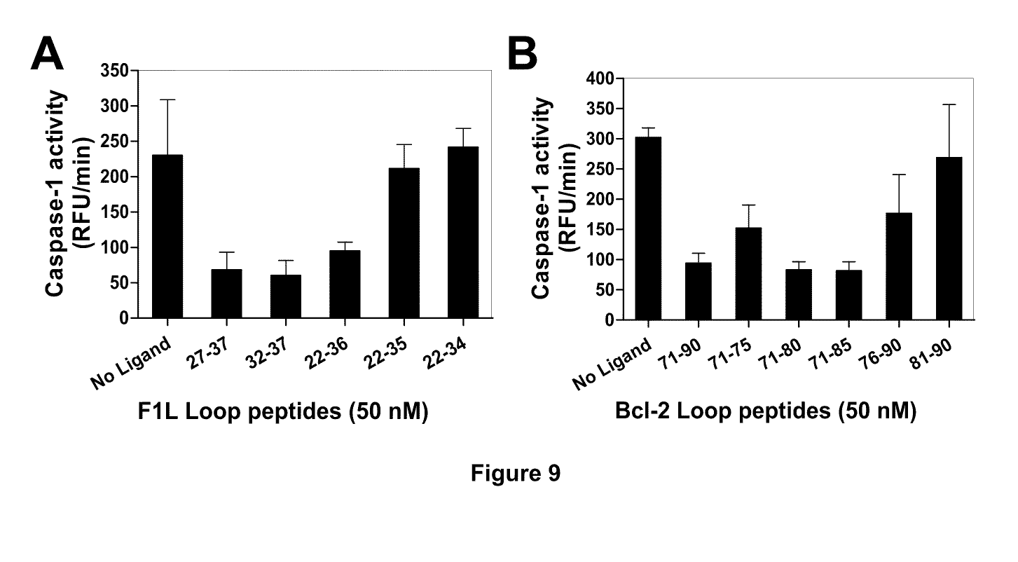

FIG. 9 shows that synthetic F1L (32-37) and Bcl-2 (71-80) peptides inhibit the NLRP1 inflammasome. Reactions contained His-NLRP1 (8.5 nM), pro-caspase-1 (8.5 nM), 0.25 mM ATP, 0.5 mM Mg2+, 0.1 μg/ml MDP and various lengths of synthetic peptides of (A) F1L and (B) Bcl-2 (50 nM). Caspase-1 activity was measured after 60 minutes by hydrolysis of Ac-WEHD-AMC substrate (20 μM), expressing data as mean±SD, n=3. Caspase-1 activity was measured after 60 minutes by hydrolysis of Ac-WEHD-AMC substrate (20 μM), expressing data as mean±SD, n=3.

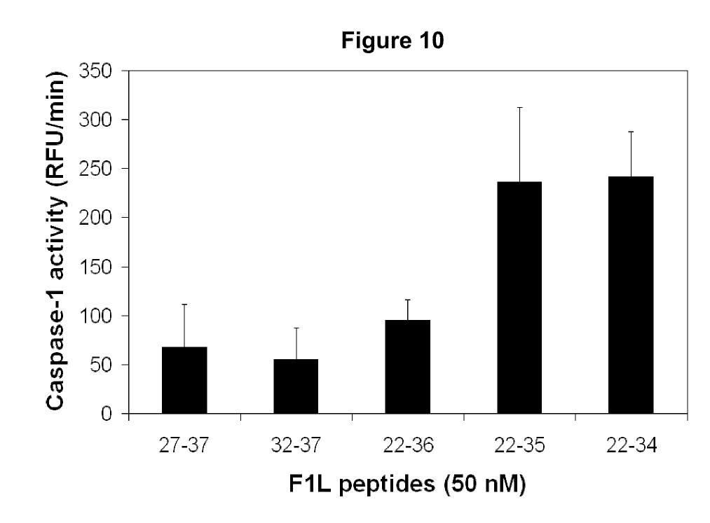

FIG. 10 shows data indicating that recombinant F1L (residues 32-37) provides maximum suppression of the in vitro reconstituted NLRP1 inflammasome. Reactions contained His-NLRP1 (8.5 nM), pro-caspase-1 (8.5 nM), 0.25 mM ATP, 0.5 mM Mg2+, 0.1 μg/ml MDP and various constructs of recombinant F1L (50 nM). Caspase-1 activity was measured after 60 minutes by hydrolysis of Ac-WEHD-AFC substrate (20 μM), expressing data as mean±SD, n=3.

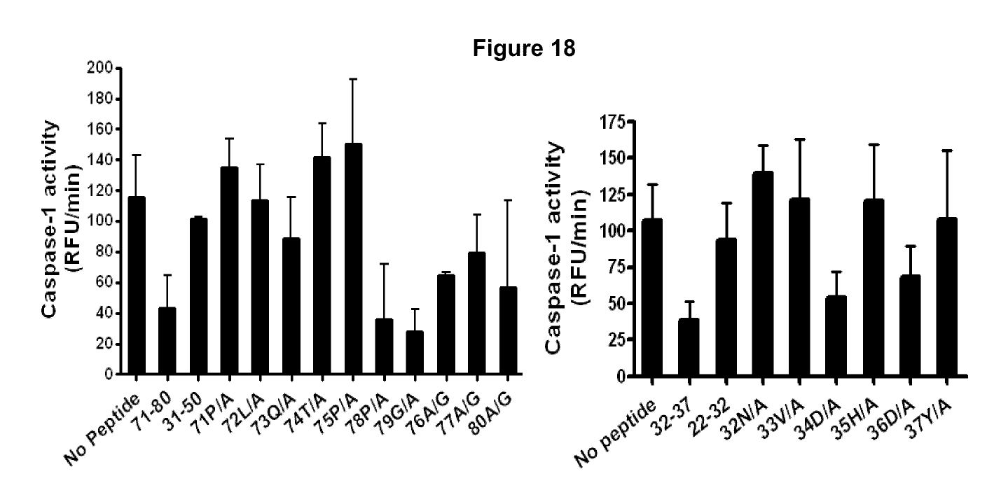

FIG. 18 shows data from the alanine scanning of Bcl-2 71-80 and F1L 32-37 peptides. Reactions contained His6-NLRP1 (8.5 nM), pro-Caspase-1 (8.5 nM), 0.25 mM ATP, 0.5 mM Mg2+, 0.1 μg/ml MDP and various (A) Bcl-2 or (B) F1L Loop mutant peptides (50 nM) for which each amino acid was consecutively substituted by Alanine. Caspase-1 activity was measured after 60 minutes by hydrolysis of Ac-WEHD-AMC substrate (20 μM), expressing data as mean±SD, n=3.

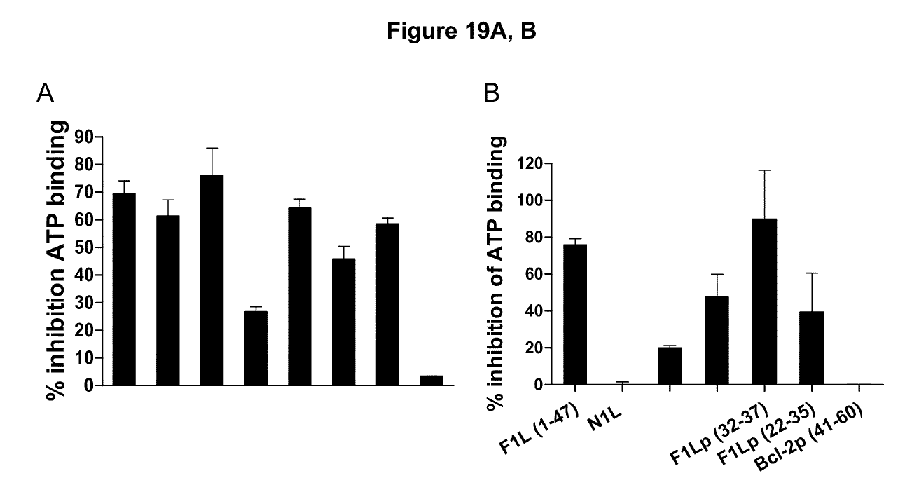

FIG. 19 shows that Bcl-2 and F1L peptides inhibit ATP binding to NLRP1. (A) ATP binding by NLRP1 was measured in presence or absence of peptides. Reactions contained His6-NLRP1 (0.125 μM), GST-Bcl-2 protein or Bcl-2 loop peptides (2 or 20 μM), 1 μM MDP[LD], FITC-ATP analog (10 nM) and Mg2+ (0.5 mM). FITC-ATP binding was analyzed by FPA (n=3), measuring milliPolars [mP], and values corrected for non-specific FITC-ATP binding as determined by competition with excess unlabeled ATP. The percentage inhibition was determined compared to NLRP1 incubated only with MDP-LD (mean±SD). (B) His6-NLRP1 (0.125 μM) was incubated for 5 min on ice in the presence of F1L 1-47 (2 μM), N1L, or F1L peptides 22-47 (0.5 or 2 μM) or 32-37, or Bcl-2 peptide 41-60 (2 μM). The mixture was then incubated for additional 5 min with 1 μM MDP, FL-conjugated ATP analog (10 nM) and Mg2+ (0.5 mM). ATP binding was analyzed by fluorescence polarization (n=3 milliPolars [mP]), and the percentage of inhibition was determined vs NLRP1 incubated only with MDP (mean±SD; n=3).

TABLE 5
Synthetic F1L peptides

| | |
|---|---|
| Peptide | |
| length | Amino acid sequence |

25′mers

| | |
|---|---|
| 7-31 | CNNIVDYVDDIDNGIVQDIEDEASN |
| | (SEQ ID NO: 23) |
| 12-36 | DYVDDIDNGIVQDIEDEASNNVDHD |
| | (SEQ ID NO: 24) |
| 17-41 | IDNGIVQDIEDEASNNVDHDYVYPL |
| | (SEQ ID NO: 25) |
| 22-47 | VQDIEDEASNNVDHDYVYPLPENMVY |
| | (SEQ ID NO: 26) |

Short peptides

| | |
|---|---|
| 22-42 | VQDIEDEASNNVDHDYVYPLP |
| | (SEQ ID NO: 27) |
| 22-37 | VQDIEDEASNNVDHDY |
| | (SEQ ID NO: 28) |
| 22-32 | VQDIEDEASNN |
| | (SEQ ID NO: 29) |
| 27-37 | DEASNNVDHDY |
| | (SEQ ID NO: 30) |
| 32-37 | NVDHDY |
| | (SEQ ID NO: 34) |
| 22-35 | VQDIEDEASNNVDH |
| | (SEQ ID NO: 31) |
| 22-34 | VQDIEDEASNNVD |
| | (SEQ ID NO: 32) |
| 22-33 | VQDIEDEASNNV |
| | (SEQ ID NO: 33) |

Alanine substituted 6′mers

| | |
|---|---|
| N32A | AVDHDY |
| | (SEQ ID NO: 106) |
| V33A | NADHDY |
| | (SEQ ID NO: 107) |
| D34A | NVAHDY |
| | (SEQ ID NO: 104) |
| H35A | NVDADY |
| | (SEQ ID NO: 108) |
| D36A | NVDHAY |
| | (SEQ ID NO: 182) |
| Y37A | NVDHDA |
| | (SEQ ID NO: 109) |

FAM labeled peptides

| | |
|---|---|
| 7-31 | FAM-CNNIVDYVDDIDNGIVQDIEDEASN |
| | (SEQ ID NO: 110) |
| 12-36 | FAM-DYVDDIDNGIVQDIEDEASNNVDHD |
| | (SEQ ID NO: 111) |
| 17-41 | FAM-IDNGIVQDIEDEASNNVDHDYVYPL |
| | (SEQ ID NO: 112) |
| 22-47 | FAM-VQDIEDEASNNVDHDYVYPLPENMVY |
| | (SEQ ID NO: 113) |

TABLE 6
Synthetic Bcl-2 Loop peptides

| Peptide | |
|---|---|
| length | Amino acids sequence |
| Loop | VGAAPPGAAPAPGIFSSQPGHTPHPAASRDPVARTSPLQT |
| 35-83 | PAAPGAAAG |
| | (SEQ ID NO: 161) |
| BH4 | YDNREIVMKYIHYKLSQRGYEW |
| domain | (SEQ ID NO: 162) |

Loop 20′mers

| | |
|---|---|
| 31-50 | DAGDVGAAPPGAAPAPGIFS |
| | (SEQ ID NO: 163) |
| 41-60 | GAAPAPGIFSSQPGHTPHPA |
| | (SEQ ID NO: 164) |
| 51-70 | SQPGHTPHPAASRDPVARTS |
| | (SEQ ID NO: 165) |
| 61-80 | ASRDPVARTSPLQTPAAPGA |
| | (SEQ ID NO: 166) |
| 71-90 | PLQTPAAPGAAAGPALSPVP |
| | (SEQ ID NO: 167) |

Loop 15′mers

| | |
|---|---|
| 71-85 | PLQTPAAPGAAAGPA |
| | (SEQ ID NO: 168) |
| 76-90 | AAPGAAAGPALSPVP |
| | (SEQ ID NO: 169) |

Loop 10′mers

| | |
|---|---|
| 71-80 | PLQTPAAPGA |
| | (SEQ ID NO: 114) |
| 81-90 | AAGPALSPVP |
| | (SEQ ID NO: 170) |

Loop 5′mer

| | |
|---|---|
| 71-75 | PLQTP |
| | (SEQ ID NO: 171) |

TABLE 7
Synthetic Bcl-2 Loop peptides
Alanine substituted 10′ mers

| Peptide length | Amino acids sequence |
|---|---|
| 71P/A | ALQTPAAPGA |
| | (SEQ ID NO: 172) |
| 72L/A | PAQTPAAPGA |
| | (SEQ ID NO: 173) |
| 73Q/A | PLATPAAPGA |
| | (SEQ ID NO: 174) |
| 74T/A | PLQAPAAPGA |
| | (SEQ ID NO: 175) |
| 75P/A | PLQTAAAPGA |
| | (SEQ ID NO: 176) |
| 78P/A | PLQTPAAAGA |
| | (SEQ ID NO: 177) |
| 79G/A | PLQTPAAPAA |
| | (SEQ ID NO: 178) |
| 76A/G | PLQTPGAPGA |
| | (SEQ ID NO: 179) |
| 77A/G | PLQTPAGPGA |
| | (SEQ ID NO: 180) |
| 80A/G | PLQTPAAPGG |
| | (SEQ ID NO: 181) |

### J. SEQUENCES

1.

SEQ ID NO: 1

atgttgtcgatgtttatgtgtaataatatcgtagattatgtagatgatat
agataatggtatagtacaggatatagaagatgaggctagcaataatgttg
atcacgactatgtatatccacttccagaaaatatggtatatagatttgac
aagtccactaacatactcgattatctatcaacggaacgggaccatgtaat
gatggctgttcgatactatatgagtaaacaacgtttagacgacttgtata
gacagttgcccacaaagactagatcatatatagatattatcaacatatat
tgtgataaagttagtaatgattataatagggacatgaatatcatgtatga
tatggcatctacaaaatcatttacagtttatgacataaataacgaagtta
atactatactaatggataacaaggggttgggtgtaagattggcgacaatt
tcatttataaccgaattgggtagacgatgtatgaacccagtagaaacgat
aaaaatgtttactctactatcgcatactatatgcgatgattattttgtag
attatataacggacatttcaccaccagataataccatccctaacactagc
acgcgtgaatatctaaagcttattggcatcacagctatcatgtttgctac
atataaaactctcaaatacatgataggataa

2.

SEQ ID NO: 2

Met Leu Ser Met Phe Met Cys Asn Asn Ile Val Asp
Tyr Val Asp Asp Ile Asp Asn Gly Ile Val Gln Asp
Ile Glu Asp Glu Ala Ser Asn Asn Val Asp His Asp
Tyr Val Tyr Pro Leu Pro Glu Asn Met Val Tyr Arg
Phe Asp Lys Ser Thr Asn Ile Leu Asp Tyr Leu Ser
Thr Glu Arg Asp His Val Met Met Ala Val Arg Tyr
Tyr Met Ser Lys Gln Arg Leu Asp Asp Leu Tyr Arg
Gln Leu Pro Thr Lys Thr Arg Ser Tyr Ile Asp Ile
Ile Asn Ile Tyr Cys Asp Lys Val Ser Asn Asp Tyr
Asn Arg Asp Met Asn Ile Met Tyr Asp Met Ala Ser
Thr Lys Ser Phe Thr Val Tyr Asp Ile Asn Asn Glu
Val Asn Thr Ile Leu Met Asp Asn Lys Gly Leu Gly
Val Arg Leu Ala Thr Ile Ser Phe Ile Thr Glu Leu
Gly Arg Arg Cys Met Asn Pro Val Glu Thr Ile Lys
Met Phe Thr Leu Leu Ser His Thr Ile Cys Asp Asp
Tyr Phe Val Asp Tyr Ile Thr Asp Ile Ser Pro Pro
Asp Asn Thr Ile Pro Asn Thr Ser Thr Arg Glu Tyr
Leu Lys Leu Ile Gly Ile Thr Ala Ile Met Phe Ala
Thr Tyr Lys Thr Leu Lys Tyr Met Ile Gly

3.

SEQ ID NO: 3

Met Ala His Ala Gly Arg Thr Gly Tyr Asp Asn Arg
Glu Ile Val Met Lys Tyr Ile His Tyr Lys Leu Ser
Gln Arg Gly Tyr Glu Trp Asp Ala Gly Asp Val Gly
Ala Ala Pro Pro Gly Ala Ala Pro Ala Pro Gly Ile
Phe Ser Ser Gln Pro Gly His Thr Pro His Pro Ala
Ala Ser Arg Asp Pro Val Ala Arg Thr Ser Pro Leu
Gln Thr Pro Ala Ala Pro Gly Ala Ala Ala Gly Pro
Ala Leu Ser Pro Val Pro Pro Val Val His Leu Thr
Leu Arg Gln Ala Gly Asp Asp Phe Ser Arg Arg Tyr
Arg Arg Asp Phe Ala Glu Met Ser Ser Gln Leu His
Leu Thr Pro Phe Thr Ala Arg Gly Arg Phe Ala Thr
Val Val Glu Glu Leu Phe Arg Asp Gly Val Asn Trp
Gly Arg Ile Val Ala Phe Phe Glu Phe Gly Gly Val
Met Cys Val Glu Ser Val Asn Arg Glu Met Ser Pro
Leu Val Asp Asn Ile Ala Leu Trp Met Thr Glu Tyr
Leu Asn Arg His Leu His Thr Trp Ile Gln Asp Asn
Gly Gly Trp Val Gly Ala Leu Gly Asp Val Ser Leu
Gly

4.

SEQ ID NO: 4

Met Ala His Ala Gly Arg Thr Gly Tyr Asp Asn Arg
Glu Ile Val Met Lys Tyr Ile His Tyr Lys Leu Ser
Gln Arg Gly Tyr Glu Trp Asp Ala Gly Asp Val Gly
Ala Ala Pro Pro Gly Ala Ala Pro Ala Pro Gly Ile
Phe Ser Ser Gln Pro Gly His Thr Pro His Pro Ala
Ala Ser Arg Asp Pro Val Ala Arg Thr Ser Pro Leu
Gln Thr Pro Ala Ala Pro Gly Ala Ala Ala Gly Pro
Ala Leu Ser Pro Val Pro Pro Val Val His Leu Thr
Leu Arg Gln Ala Gly Asp Asp Phe Ser Arg Arg Tyr
Arg Arg Asp Phe Ala Glu Met Ser Ser Gln Leu His
Leu Thr Pro Phe Thr Ala Arg Gly Arg Phe Ala Thr
Val Val Glu Glu Leu Phe Arg Asp Gly Val Asn Trp
Gly Arg Ile Val Ala Phe Phe Glu Phe Gly Gly Val
Met Cys Val Glu Ser Val Asn Arg Glu Met Ser Pro
Leu Val Asp Asn Ile Ala Leu Trp Met Thr Glu Tyr
Leu Asn Arg His Leu His Thr Trp Ile Gln Asp Asn
Gly Gly Trp Asp Ala Phe Val Glu Leu Tyr Gly Pro
Ser Met Arg Pro Leu Phe Asp Phe Ser Trp Leu Ser
Leu Lys Thr Leu Leu Ser Leu Ala Leu Val Gly Ala
Cys Ile Thr Leu Gly Ala Tyr Leu Gly His Lys

| | |
|---|---|
| 5. | |
| GRKKRRQRPPQ | SEQ ID NO: 5 |

| | |
|---|---|
| 6. | |
| RQIKIWFQNRRMKWKK | SEQ ID NO: 6 |

| | |
|---|---|
| 7. | |
| RQIAIWFQNRRMKWAA | SEQ ID NO: 7 |

| | | |
|---|---|---|
| 8. | SEQ ID NO: 8 | RKKRRQRRR |

| | | |
|---|---|---|
| 9. | SEQ ID NO: 9 | TRSSRAGLQFPVGRVHRLLRK |

| | | |
|---|---|---|
| 10. | SEQ ID NO: 10 | GWTLNSAGYLLGKINKALAALAKKIL |

| | | |
|---|---|---|
| 11. | SEQ ID NO: 11 | KLALKLALKALKAALKLA |

| | | |
|---|---|---|
| 12. | SEQ ID NO: 12 | AAVALLPAVLLALLAP |

| | | |
|---|---|---|
| 13. | SEQ ID NO: 13 | VPMLKPMLKE |

| | | |
|---|---|---|
| 14. | SEQ ID NO: 14 | MANLGYWLLALFVTMWTDVGLCKKRPKP |

| | | |
|---|---|---|
| 15. | SEQ ID NO: 15 | LLIILRRRIRKQAHAHSK |

| | | |
|---|---|---|
| 16. | SEQ ID NO: 16 | KETWWETWWTEWSQPKKKRKV |

| | | |
|---|---|---|
| 17. | SEQ ID NO: 17 | RGGRLSYSRRRFSTSTGR |

| | | |
|---|---|---|
| 18. | SEQ ID NO: 18 | SDLWEMMMVSLACQY |

| | | |
|---|---|---|
| 19. | SEQ ID NO: 19 | TSPLNIHNGQKL |

| | | |
|---|---|---|
| 20. | SEQ ID NO: 20 | UACUGUUUGUCAUGCCACUUCUGAU |

| | | |
|---|---|---|
| 21. | SEQ ID NO: 21 | RRRRRRRRR |

| | | |
|---|---|---|
| 22. | SEQ ID NO: 22 | RQPKIWFPNRRKPWKK |

1. An isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR.

2. The isolated peptide of claim 1, wherein the amino acid sequence comprises amino acid residues 32-37 of SEQ ID NO:2.

3. The isolated peptide of claim 2, wherein the amino acid sequence consists of amino acid residues 32-37 of SEQ ID NO:2.

4. The isolated peptide of claim 1, wherein the amino acid sequence comprises amino acid residues 27-37, 22-37, or 22-47 of SEQ ID NO:2, or a fragment thereof of at least 6 amino acids.

5. The isolated peptide of claim 1, wherein the amino acid sequence consists of amino acid residues 27-37, 22-37, or 22-47 of SEQ ID NO:2, or a fragment thereof of at least 6 amino acids.

6. The isolated peptide of claim 1, wherein the amino acid sequence does not consist of an amino acid segment of a naturally occurring protein other than F1L.

7. The isolated peptide of claim 1, wherein the peptide does not consist of an amino acid segment of a naturally occurring protein other than F1L.

8. The isolated peptide of claim 1, wherein the methionine at position 45 is oxidized.

9. The isolated peptide of claim 1, wherein the methionine at position 45 is reduced.

10. The isolated peptide of claim 1, wherein the amino acid sequence comprises amino acid residues 71-80 of SEQ ID NO:3.

11. The isolated peptide of claim 10, wherein the amino acid sequence consists of amino acid residues 71-80 of SEQ ID NO:3.

12. The isolated peptide of claim 1, wherein the amino acid sequence comprises amino acid residues 71-80, 71-90, 71-100, 65-80, 60-80, 60-90, or 60-100 of SEQ ID NO:3, or a fragment thereof of at least 6 amino acids.

13. The isolated peptide of claim 1, wherein the amino acid sequence consists of amino acid residues 71-80, 71-90, 71-100, 65-80, 60-80, 60-90, or 60-100 of SEQ ID NO:3, or a fragment thereof of at least 6 amino acids.

14. The isolated peptide of claim 1, wherein the amino acid sequence does not consist of an amino acid segment of a naturally occurring protein other than Bcl-2.

15. The isolated peptide of claim 1, wherein the peptide does not consist of an amino acid segment of a naturally occurring protein other than Bcl-2.

16. The isolated peptide of claim 1, wherein the NLR is NLRP1 or NLRP3.

17. The isolated peptide of claim 1, wherein the isolated peptide inhibits the binding of ATP to the NLR.

18. The isolated peptide of claim 1, wherein the peptide is conjugated to a tag.

19. The isolated peptide of claim 18, wherein the tag is a label, biotin, streptavidin, avidin, a fluorochrome, a fluorescent label, a label enzyme, a tag, or a combination.

20. The isolated peptide of claim 1, wherein the isolated peptide comprises an internalization sequence.

21. The isolated peptide of claim 20, wherein the internalization sequence comprises HIV-Tat protein, Drosophila antennapedia protein, poly-arginine, poly-L-arginine, poly-D-arginine, or a combination.

22. A composition comprising any of the peptides of claim 1 and a pharmaceutically acceptable carrier.

23. A method of identifying an inhibitor of inflammation, comprising
preparing a sample comprising a Nod-Like Receptor (NLR), an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and a candidate agent,
detecting the binding of the isolated peptide to the NLR,
wherein a decrease in the binding of the isolated peptide and the NLR as compared to a control is an indication that the candidate agent is an inhibitor of inflammation.

24. The method of claim 23, wherein the amino acid sequence comprises amino acid residues 22-47 of SEQ ID NO:2, or a fragment thereof of at least 6 amino acids.

25. The method of claim 23, wherein the amino acid sequence comprises amino acid residues 71-80 of SEQ ID NO:3, or a fragment thereof of at least 6 amino acids.

26. The method of claim 23, wherein the NLR is NLRP1 or NLRP3.

27. The method of claim 23, wherein the isolated peptide inhibits the binding of ATP to the NLR.

28. The method of claim 23, wherein the peptide is conjugated to a tag.

29. The method of claim 28, wherein the tag is a label, biotin, streptavidin, avidin, a fluorochrome, a fluorescent label, a label enzyme, a tag, or a combination.

30. The method of claim 23, wherein the peptide is conjugated to a fluorochrome, wherein binding of the isolated peptide to the NLR is measured by fluorescent polarization assay (FPA).

31. The method of claim 23, wherein binding of the isolated peptide to the NLR is measured by time resolved fluorescent resonance energy transfer (TR-FRET).

32. The method of claim 23, wherein binding of the isolated peptide to the NLR is measured by scintillation proximity assay (SPA).

33. The method of claim 23, wherein the inflammation is acute.

34. The method of claim 23, wherein the inflammation is chronic.

35. A method of identifying an inhibitor of inflammation, comprising
preparing a sample comprising a Nod-Like Receptor (NLR), any of the isolated peptides of claim 1, and a candidate agent,
detecting the binding of the isolated peptide to the NLR,
wherein a decrease in the binding of the isolated peptide and the NLR as compared to a control is an indication that the candidate agent is an inhibitor of inflammation.

36. A method of treating inflammation in a subject, comprising administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR.

37. The method of claim 36, wherein the amino acid sequence comprises amino acid residues 22-47 of SEQ ID NO:2, or a fragment thereof of at least 6 amino acids.

38. The method of claim 36, wherein the amino acid sequence comprises amino acid residues 71-80 of SEQ ID NO:3, or a fragment thereof of at least 6 amino acids.

39. The method of claim 36, wherein the inflammation is caused or exacerbated by IL-1β secretion.

40. The method of claim 39, wherein the IL-1β secretion is activated by inflammasome-mediated caspase-1 activation.

41. The method of claim 36, wherein the inflammation is caused or exacerbated by Vitiligo.

42. The method of claim 36, wherein the isolated peptide comprises an internalization sequence.

43. The method of claim 42, wherein the internalization sequence comprises HIV-Tat protein, Drosophila antennapedia protein, poly-arginine, poly-L-arginine, poly-D-arginine, or a combination.

44. The method of claim 36, wherein the inflammation is acute.

45. The method of claim 36, wherein the inflammation is chronic.

46. A method of treating inflammation in a subject, comprising administering to the subject any of the isolated peptides of claim 1.

47. A method of treating Bacillus anthracis infection or ameliorating Bacillus anthracis symptoms in a subject, comprising administering to the subject an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR.

48. The method of claim 47, wherein the amino acid sequence comprises amino acid residues 22-47 of SEQ ID NO:2, or a fragment thereof of at least 6 amino acids.

49. The method of claim 47, wherein the amino acid sequence comprises amino acid residues 71-80 of SEQ ID NO:3, or a fragment thereof of at least 6 amino acids.

50. The method of claim 47, wherein the isolated peptide comprises an internalization sequence.

51. The method of claim 50, wherein the internalization sequence comprises HIV-Tat protein, Drosophila antennapedia protein, poly-arginine, poly-L-arginine, poly-D-arginine, or a combination.

52. A method of treating Bacillus anthracis infection or ameliorating Bacillus anthracis symptoms in a subject, comprising administering to the subject any of the isolated peptides of claim 1.

53. A method of detecting Nod-Like Receptors (NLRs), comprising
(a) bringing into contact a sample and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, wherein the peptide is conjugated to a first tag, wherein the first tag is in a binding pair with a second tag,
(b) prior to, simultaneous with, or following step (a), bringing into contact the peptide and a surface substrate comprising the second tag, wherein the conjugated peptide binds to the second tag, and
(c) detecting NLR associated with the surface substrate.

54. The method of claim 53, wherein the amino acid sequence comprises amino acid residues 22-47 of SEQ ID NO:2, or a fragment thereof of at least 6 amino acids.

55. The method of claim 53, wherein the amino acid sequence comprises amino acid residues 71-80 of SEQ ID NO:3, or a fragment thereof of at least 6 amino acids.

56. The method of claim 53, wherein the first tag is biotin and the second tag is streptavidin or avidin.

57. The method of claim 53, wherein the support surface comprises a bead, a plate, or a multi-well plate.

58. The method of claim 53, wherein detecting the NLR is accomplished via an enzyme-linked immunosorbent assay.

59. A method of identifying inhibitory sites on Nod-Like Receptors (NLRs), comprising
bringing into contact a NLR and an isolated peptide comprising an amino acid sequence having at least 70% identity to the sequence set forth in SEQ ID NO:2, SEQ ID NO:3, or SEQ ID NO:4 that can bind to a Nod-Like Receptor (NLR), or a fragment the sequence set forth in SEQ ID NO:2, SEQ ID NO:3, or SEQ ID NO:4 of at least 6 amino acids in length that can bind to a NLR, and
detecting the location where the peptide binds the NLR.

60. The method of claim 59, wherein the location where the peptide binds the NLR is detected using nuclear magnetic resonance.

61. The method of claim 59, wherein the location where the peptide binds the NLR is detected using x-ray crystallography to identify.

62. A method of treating a subject, comprising administering to the subject an isolated peptide comprising an amino acid sequence having at least 70% identity to the sequence set forth in SEQ ID NO:2, SEQ ID NO:3, or SEQ ID NO:4 that can compete with F1L, Bcl-2, or both for binding a Nod-Like Receptor (NLR) and, or a fragment the sequence set forth in SEQ ID NO:2, SEQ ID NO:3, or SEQ ID NO:4 of at least 6 amino acids in length that can compete with F1L, Bcl-2, or both for binding a NLR, wherein the subject is suffering from a viral disease.

63. The isolated peptide of claim 1, wherein the amino acid sequence comprises amino acids 32-37 of SEQ ID NO:2, amino acids 32-37 of SEQ ID NO:2 where one or both of amino acids 34 and 36 is substituted independently with any amino acid, or amino acids 71-80 of SEQ ID NO:3 where one or both of amino acids 34 and 36 is substituted independently with E, A, G, V, L, F, I, W, or P.

64. The isolated peptide of claim 1, wherein the amino acid sequence comprises amino acids 71-80 of SEQ ID NO:3, amino acids 71-80 of SEQ ID NO:3 where one or more of amino acids 72, 73, 76, 77, 78, 79, and 80 is substituted independently with any amino acid, or amino acids 71-80 of SEQ ID NO:3 where one or more of amino acids 72, 73, 76, 77, 78, 79, and 80 is substituted independently with A, G, I, V, F, W, or P (for amino acid 72), N, R, A, G, V, L, F, I, W, or P (for amino acid 73), G, V, L, F, I, W, or P (for amino acid 76), G, V, L, F, I, W, or P (for amino acid 77), A, G, V, L, F, I, or W (for amino acid 78), A, V, L, F, I, or W (for amino acid 79), and G, V, L, F, I, W, or P (for amino acid 80).
